(19) United States
(12) Patent Application Publication
(10) Pub. No.: US 2024/0306983 A1
(43) Pub. Date: Sep. 19, 2024

(54) SYSTEM AND METHOD FOR DETERMINING AROUSALS AND AROUSAL-ASSOCIATED EVENTS OF A SLEEP STUDY USING NON-BRAIN BODY SIGNALS OR WITHOUT REQUIRING BRAIN SIGNALS

(71) Applicant: Nox Medical ehf, Reykjavik (IS)

(72) Inventors: Sveinbjorn HOSKULDSSON, Reykjavik (IS); Jon Skirnir AGUSTSSON, Reykjavik (IS); Sigurdur Ægir JONSSON, Reykjavik (IS); Ernir ERLINGSSON, Reykjavik (IS); Hrafnkell Zagross ZAHAWI, Reykjavik (IS)

(21) Appl. No.: 18/608,526

(22) Filed: Mar. 18, 2024

Related U.S. Application Data
(60) Provisional application No. 63/490,984, filed on Mar. 17, 2023.
(60) Provisional application No. 63/613,562, filed on Dec. 21, 2023.

(51) Int. Cl. A61B 5/00; A61B 5/08; A61B 5/113; A61B 5/369

(52) U.S. Cl. CPC A61B 5/4818; A61B 5/0809; A61B 5/0826; A61B 5/113; A61B 5/369; A61B 5/4809; A61B 5/7264

(57) ABSTRACT

Systems, devices, and methods are provided for determining an arousal or arousal-associated event in a sleep study of a subject. The method includes obtaining data from one or more body signals, the one or more body signals being non-brain signals, and determining an arousal or arousal-associated event of the subject using the data from one or more body signals. In a preferred embodiment, the one or more body signals include data obtained from one or more RIP belts.

### CROSS-REFERENCE TO RELATED APPLICATIONS

This application claims the benefit of and priority to United States Provisional Patent Application Serial Nos. 63/490,984, filed on Mar. 17, 2023, and 63/613,562, filed on Dec. 21, 2023, both of which are incorporated herein by reference in their entirety.

### FIELD OF THE DISCLOSURE

The present disclosure relates to a system, apparatuses, and a method for determining arousals and sleep stages of a subject, and particularly for determining arousals and sleep stages based on signals obtained from the body of the subject without necessarily being signals obtained from the brain or heart of the subject.

### BACKGROUND

Clinical sleep studies of different types have been developed. Such studies have either focused on measuring or identifying a specific sleep disorder or have been more general for measuring the overall sleep profile along with the signals necessary to confirm or exclude different sleep disorders.

Polysomnography (PSG) is a general sleep study that records various physiological signals. A PSG is generally considered a complicated study and usually requires professional assistance by certified or credential technologists to setup, perform, and monitor the PSG. PSG includes simultaneous recording of multiple signals, such as Electroencephalography (EEG), Electrooculography (EOG), Electromyography (EMG), Electrocardiography (ECG), Respiratory Flow, Respiratory Effort, Oximetry, Body Position, and/or more to achieve the required accuracy.

During a PSG the electroencephalography (EEG) signals are obtained from the head of a subject for determining sleep stages of the subject. The time people spend in bed can normally be divided into certain periods or stages of Rapid Eye Movement (REM) sleep, Non-rapid eye movement sleep (Non-REM or NREM) sleep, and occasional Wake periods. Standard PSG allows further classification of the NREM periods on different levels of sleep including N1, N2, and N3, with N1 being the shallowest, then N2, and finally N3. The N3 period is often referred to as deep sleep or Slow Wave Sleep due to the slow EEG signals that are characteristic of this period. The sleep stages are often presented in a graph with the X axis labeled with the time of day and the Y axis showing 5 values, Wake, REM, N1, N2, N3. A line may then be plotted showing the sleep stage of the subject at different times of the night or sleep study period. Such a graph is called hypnogram and is the standard presentation of the sleep profile used in PSG studies.

The sleep indexes, which are derived directly from the sleep study signals, often include an expansive collection of indices derived from the sleep study. These indices may include, but are not limited to:
Arousal Index: The number of arousals per hour of sleep.
Apnea-Hypopnea Index: The number of complete breathing cessations (apneas), and severely restricted breathing (hypopnea) events per hour of sleep.
Oxygen Desaturation Index: The number of blood oxygen desaturation events per hour of sleep.
Limb Movement Index: The number of limb movement events per hour of sleep.
Periodic Limb Movement Index: The number of periodic limb movement events per hour of sleep.
Total Sleep Time: The total time duration of sleep during the sleep study.
Wake After Sleep Onset: The total time duration of wake periods from the first moment of sleep to the last moment of sleep.
Position: A measurement of the periods a patient is sleeping in a supine, prone, left-side, or right-side positions.

Electroencephalography (EEG) is typically based on electrodes placed on the scalp of the subject. The clinical standards for PSG require that the recording of EEG signals is done with electrodes located on parts of the head typically covered in hair. But a patient or subject generally can't or has difficulty applying the sleep study electrodes on himself, or at least has difficulty applying the sleep study electrodes on himself correctly. Therefore the patient must be assisted by a nurse or technician. For this reason, most PSG studies are done in a clinic, as the patient needs to be prepared for the sleep study around the time he goes to bed.

Another common type of sleep study is Home Sleep Apnea Testing (HSAT). HSAT generally only focuses on respiratory parameters and oxygen saturation for diagnosing sleep apnea and sleep disordered breathing. HSAT does however not require EEG electrodes on the head or sensors that the patient can't place on him himself. Therefore, the most common practice in HSAT is to hand the HSAT system to the patient over-the-counter in the clinic or send the HSAT system by mail to the patient and have the patient handle the hookup or placement of the HSAT system to himself. This is a highly cost-efficient process for screening for sleep apnea. However, this practice has the drawback that the sleep stages, including time of sleep/wake periods is missing. It is therefore the risk of HSAT not performed in a clinic that the patient was indeed not sleeping during the whole recording time. But as this may not be known to the technician scoring the data from the HST after the study, there is the risk that this could affect the clinical decision on the severity of the sleep apnea. It would therefore be preferred to have some prediction or determination of the sleep stages of the subject to improve the accuracy of the diagnoses. But as noted above, doing a standard EEG on the patient during the HSAT would be impractical or impossible in a home-type setting, or too expensive. Although current HSAT systems and methods can provide an accurate indication of total sleep time (TST), for example, as described in US 2021/0085242A1, as described below they suffer from the significant inability to determine arousal or arousal-associated events, and therefore current HSAT systems and methods are not able to correctly classify all three types of apnea/hypopnea events of a respiratory event index (REI).

The American Academy of Sleep Medicine (AASM) defines what signals are recorded in each type of sleep studies and how the signals are interpreted to make a diagnosis. To diagnose sleep apnea the main outcome of interest is the apnea hypopnea index (AHI), which is a number that counts the number of apnea and hypopnea events occurring during sleep. According to the AASM, an “apnea” is a respiratory event that is defined by a 90% reduction in airflow from baseline lasting at least 10 seconds. Further, according to the AASM, a “hypopnea” is defined as respiratory events where the reduction in airflow is between 30% and 90% from baseline and the reduction in airflow is associated with a 3% or 4% drop in blood oxygen saturation and/or a cortical arousal.

For example, the published “Rules for Scoring Respiratory Events in Sleep”, as updated in 2007, the AASM further defines an apnea event, stating: “Hypopnea in adults is scored when the peak signal excursions drop by 30% of pre-event baseline using nasal pressure (diagnostic study), PAP device flow (titration study), or an alternative sensor, for ≥10 seconds in association with either ≥3% arterial oxygen desaturation or an arousal.” (Berry R B; Budhiraja R; et al. Rules for scoring respiratory events in sleep: update of the 2007 AASM Manual for the Scoring of Sleep and Associated Events. J Clin Sleep Med 2012; 8(5):597-619, at 597 (emphasis added); see also Malhotra R K, et al.; American Academy of Sleep Medicine Board of Directors. Polysomnography for obstructive sleep apnea should include arousal-based scoring: an American Academy of Sleep Medicine position statement. J Clin Sleep Med. 2018; 14(7):1245-1247; Rosen I M, et al.; American Academy of Sleep Medicine Board of Directors. Clinical use of a home sleep apnea test: an updated American Academy of Sleep Medicine position statement. J Clin Sleep Med. 2018; 14(12):2075-2077; and Riha R L, et al. ERS technical standards for using type III devices (limited channel studies) in the diagnosis of sleep disordered breathing in adults and children. Eur Respir J 2023; 61: 2200422 [DOI: 10.1183/13993003.00422-2022].

To aid in the understanding of current scoring rules for respiratory events in sleep studies that are used and accepted in the field and promulgated by authorities, current and relevant scoring rules of the AASM are provided below.

### AASM Scoring of Apneas (PSG)

From the AASM manual for the scoring of sleep and associated events polysomnography (PSG) scoring rules for apneas (v2.6, “C. Scoring of Apneas”):

### Scoring of Apneas

1. Score a respiratory event as an apnea when BOTH of the following criterial are met:
   a. There is a drop in the peak signal excursion by 290% of pre-event baseline using an oronasal thermal sensor (diagnostic study), PAP device flow (titration study) or an alternative apnea sensor (diagnostic study).
   b. The duration of the 290% drop in sensor signal is ≥10 seconds.
2. Score an apnea as “obstructive” if it meets apnea criteria and is associated with continued or increased inspiratory effort throughout the entire period of absent airflow.
3. Score an apnea as “central” if it meets apnea criteria and is associated with absent inspiratory effort throughout the entire period of absent airflow.
4. Score an apnea as “mixed” if it meets apnea criteria and is associated with absent inspiratory effort in the initial portion of the event, followed by resumption of inspiratory effort in the second portion of the event.

With reference to FIG. 1, the relevant section of the AASM manual for the scoring of apneas notes:
Note 1: Identification of an apnea does not require a minimum desaturation criterion.
Note 2: If a portion of a respiratory event that would otherwise meet criteria for a hypopnea meets criterial for apnea, the entire event should be scored as an apnea. (For example, as shown in FIG. 1, the longer duration, depicted by the white arrows in the nasal pressure channel, meets airflow criteria for a hypopnea, whereas the shorter duration, depicted by the black arrows in the oronasal thermal airflow channel, meets airflow criteria for an apnea. The even would be scored as an apnea.)
Note 3: If the apnea or hypopnea event begins or ends during an epoch that is scored as sleep, then the corresponding respiratory event can be scored and included in the computation of the apnea-hypopnea index (AHI). This situation usually occurs when an individual has a high AHI with events occurring so frequently that sleep is severely disrupted, and epochs may end up being scored as wake even though <15 seconds of sleep is present during the epoch containing that portion of the respiratory event. However, if the apnea of hypopnea occurs entirely during an epoch scored as wake, it should not be scored or counted towards the apnea-hypopnea index because of the difficulty of defining a denominator in this situation. If these occurrences are a prominent feature of the polysomnogram and/or interfere with sleep onset, their presence should be mentioned in the narrative summary of the study.
Note 4: For alternative apnea sensors see Technical Specifications for adults (A.2).
Note 5: There is not sufficient evidence to support a specific duration of the central and obstructive components of a mixed apnea; thus, specific durations of these components are not recommended.

### AASM Scoring of Hypopneas (PSG)

From the AASM manual for the scoring of sleep and associated events polysomnography (PSG) scoring rules for hypopneas (v2.6, “D. Scoring of Hypopneas”)

### Scoring of Hypopneas

### Scoring Hypopneas as Central or Obstructive Events is Option as Noted in Parameters to be Reported (II.F)

1A. Score a respiratory event as a hypopnea if ALL of the following criteria are met:
   a. The peak signal excursions drop by ≥30% of pre-event baseline using nasal pressure (diagnostic study), PAP device flow (titration study), or an alternative hypopnea sensor (diagnostic study).
   b. The duration of the ≥30% drop in signal excursion is ≥10 seconds.
   c. There is a ≥3% oxygen desaturation from pre-event baseline or the event is associated with an arousal.
1B. Score a respiratory event as a hypopnea if ALL of the following criterial are met:
   a. The peak signal excursions drop by ≥30% of pre-event baseline using nasal pressure (diagnostic study), PAP device flow (titration study), or an alternative hypopnea sensor (diagnostic study).
   b. The duration of the ≥30% drop in signal excursions is 210 seconds.
   c. There is a ≥4% oxygen desaturation from pre-event baseline.
2. If electing to score obstructive hypopneas, score a hypopnea as obstructive if ANY of the following criteria are met:
   a. There is snoring during the event.
   b. There is increased inspiratory flattening of the nasal pressure or PAP device flow signal compared to baseline breathing.
   c. There is an associated thoracoabdominal paradox that occurs during the event but not during pre-event breathing.
3. If electing to score central hypopneas, score a hypopnea as central if NONE Of the following criteria are met:
   a. There is snoring during the event.
   b. There is increased inspiratory flattening of the nasal pressure or PAP device flow signal compared to baseline breathing.
   c. There is an associated thoracoabdominal paradox that occurs during the event but not during pre-event breathing.

The relevant section of the AASM manual for the scoring of apneas notes the following:
Note 1: The criteria used to score a respiratory event as a hypopnea (either rule 1A or 1B) should be specified in the PSG report. It is the responsibility of the individual practitioner to confirm and follow the criterial that should be used for reporting to the patient's payer in order to be reimbursed and qualify the patient for therapy.
Note 2: For alternative hypopnea sensors see Technical Specifications for adults (A.4).
Note 3: Supplemental oxygen may blunt desaturation. There are currently no scoring guidelines for when a patient is on supplemental oxygen and no desaturation is noted. If the diagnostic study is performed while the individual is on supplemental oxygen, its presence should be mentioned in the narrative summary of the study.

### AASM Scoring Arousals

From the AASM manual for the scoring of sleep and associated events scoring rules for arousals (v2.6, “V. Arousal Rule”):

### Scoring Arousals

1. Score arousal during sleep stages N1, N2, N3, or R if there is an abrupt shift of EEG frequency including alpha, theta and/or frequencies greater than 16 Hz (but not spindles) that lasts at least 3 seconds, with at least 10 seconds of stable sleep preceding the change. Scoring of arousal during REM requires a concurrent increase in submental EMG lasting at least 1 second.

The relevant section of the AASM manual for the scoring of apneas notes the following:
Note 1: Arousal scoring should incorporate information from the frontal, central, and occipital derivations.
Note 2: Arousal scoring can be improved by the use of additional information in the recording such as respiratory events and/or additional EEG channels. Scoring of arousals, however, cannot be based on this additional information alone and such information does not modify any of the arousal scoring rules.
Note 3: Arousals meeting all scoring criteria but occurring during an awake epoch in the recorded time between “lights out” and “lights on” should be scored and used for computation of the arousal index.
Note 4: The 10 seconds of stable sleep required prior to scoring an arousal may begin in the preceding epoch, including a preceding epoch that is scored as stage W.
Note 5: An arousal may still be scored if it immediately precedes a transition to stage W. That is, both the arousal and transition to wake are scored.

### AASM HSAT Scoring Apneas

From the AASM manual for the scoring of sleep and associated events, home sleep apnea test (HSAT) scoring rules for apneas (v2.6, “G. HSAT Respiratory Events Rules: Scoring Apnea Utilizing Respiratory Flow and/or Effort Sensors”):
1. Score a respiratory event as an apnea when BOTH of the following criteria are met:
   a. There is a drop in the peak signal excursions by ≥90% of pre-event baseline using a recommended or alternative airflow sensor.
   b. The duration of the ≥290% drop in sensor signal is ≥10 seconds.
2. Score an apnea as “obstructive” if it meets apnea criteria and is associated with continued or increased inspiratory effort throughout the entire period of absent airflow.
3. Score an apnea as “central” if it meets apnea criteria and is associated with absent inspiratory effort throughout the entire period of absent airflow.
4. Score an apnea as “mixed” if it meets apnea criteria and is associated with absent inspiratory effort in the initial portion of the event, followed by resumption of inspiratory effort in the second portion of the event.

The relevant section of the AASM manual for the scoring of apneas notes the following:
Note 1: Identification of an apnea does not require a minimum desaturation criterion.
Note 2: If a portion of a respiratory event that would otherwise meet criteria for a hypopnea meets criteria for apnea, the entire event should be scored as an apnea.
Note 3: There is not sufficient evidence to support a specific duration of the central and obstructive components of a mixed apnea; thus, the specific durations of these components are not recommended.
Note 4: Some devices may not differentiate between different types of apneas.

### AASM HSAT Scoring Hypopneas

From the AASM manual for the scoring of sleep and associated events, home sleep apnea test (HSAT) scoring rules for hypopneas (v2.6, “H. HSAT Respiratory Events Rules: Scoring Hypopnea Utilizing Respiratory Flow and/or Effort Sensors”):
1A. If sleep is NOT recorded, score a respiratory event as a hypopnea if ALL of the following criteria are met:
   a. The peak signal excursions drop by ≥30% of pre-event baseline using a recommended or alternative airflow sensor.
   b. The duration of the ≥30% drop in signal excursions is ≥10 seconds.
   c. There is a ≥3% oxygen desaturation from pre-event baseline.
1B. If sleep is NOT recorded, score a respiratory event as a hypopnea if ALL of the following criteria are met:
   a. The peak signal excursions drop by 230% of pre-event baseline using a recommended or alternative airflow sensor.
   b. The duration of the 30% drop in signal excursions is 210 seconds.
   c. There is a 4% oxygen desaturation from pre-event baseline.
2A. If sleep IS recorded, score a respiratory event as a hypopnea if ALL of the following criteria are met:
   a. The peak signal excursions drop by ≥30% of pre-event baseline using a recommended or alternative airflow sensor.
   b. The duration of the ≥30% drop in signal excursions is ≥10 seconds.
   c. There is a ≥3% oxygen desaturation from pre-event baseline or the event is associated with an arousal.
2B. If sleep IS recorded, score a respiratory event as a hypopnea if ALL of the following criteria are met:
   a. The peak signal excursions drop by ≥30% of pre-event baseline using a recommended or alternative airflow sensor.
   b. The duration of the ≥30% drop in signal excursions is ≥10 seconds.
   c. There is a 4% oxygen desaturation from pre-event baseline.

The relevant section of the AASM manual for the scoring of apneas notes the following:
Note 1: The criteria used to score a respiratory event as a hypopnea should be s specified in the report.
Note 2: Scoring a hypopnea based on arousals is only possible if sleep is recorded.

In standard Polysomnography (PSG), a cortical arousal is currently detected as an abrupt change in electroencephalography (EEG) signals.

FIG. 2 shows an example of a scored apnea followed by a desaturation and an arousal. It is not necessary to have desaturations or arousals to score an apnea. However, it is very common that apneas are followed by a desaturation and/or an arousal. In FIG. 2 the apnea is marked with the orange square labeled “apnea” on top of the flow signal. The apnea is determined by the reduction in the amplitude of the flow signal. The desaturation event is marked by the sea green square on top of the SpO2 signal and labeled as “desaturation event”, and the arousals are the sea green squares on top of the O2-M1 signal.

FIG. 3 shows examples of scored hypopneas followed by desaturations and arousals. The hypopnea events are marked with bars drawn above the flow signal and labeled “hypopnea”, which correspond to the reduction in flow during the hypopneas is marked by the reduction in the flow signal amplitude. Desaturation events are marked on top of the SpO2 signal in the first half of the figure, and are labeled as “desaturation event”. The arousal events (labeled “arousal”) are labeled in the squares on top of the C4-M1 and C3-M2 signals.

### Respiratory Related Arousal (RERA)

Traditionally the respiratory events of apnea, hypopnea, and respiratory related arousal (RERA) are scored when diagnosing sleep apnea. Apneas are defined by the AASM as a 90% reduction in airflow. Hypopneas are defined as a 30% or more decrease in airflow followed by a 3 or 4% drop in blood oxygen saturation measured with a pulse oximeter or an arousal. RERAs are respiratory events that do not fulfill the criteria of apneas or hypopneas, and terminate in an arousal.

### SUMMARY

The inventors of the present application have identified a significant problem that the scoring of hypopnea events in a conventional Home Apnea Sleep Testing (HSAT) sleep study is limited by the fact that electroencephalography (EEG) signals are not recorded. Therefore, no cortical arousals can be directly detected, and hypopneas that end with an arousal and not a desaturation have not been detected. The inventors of the present application have found that this results in a systematic underestimation of the apnea hypopnea index (AHI), respiratory event index (REI), or respiratory disturbance index (RDI) indices from HSAT studies and may result in patients having to undergo a second sleep study or worse, being mis-diagnosed as not having sleep apnea when a more thorough sleep study such as a standard Polysomnography (PSG) would have revealed that they indeed do have sleep apnea or hypopneas.

As the popularity of home sleep apnea tests (HSAT) grows, so too does the importance of ensuring that they provide the best information possible to facilitate patient diagnosis and treatment. The inventors of the present application have identified the significant challenge to estimate a patient's Apnea Hypopnea Index (AHI) when no electroencephalography (EEG) is available. Until now, an EEG has been considered necessary to detect arousals which can influence hypopnea scoring and thus not using it can lead to a lower AHI for HSAT compared to polysomnography (PSG), potentially resulting in a patient's misdiagnosis. To address this issue, according to one embodiment, the inventors of the present application have developed and disclose herein methods and systems that predict sleep arousals using non-brain signal groups, or in other words using signals not obtained from a brain-machine-interface (BMI), or methods and systems that predict sleep arousals or arousal-associated events, including but not limited to arousal-associated hypopnea, without requiring brain signal groups.

According to one embodiment, methods and systems are disclosed herein that predict sleep arousals or arousal-associated events using non-EEG signal groups. Methods and systems are disclosed herein that predict sleep arousals without requiring EEG signal groups. Also provided, as embodiments, are methods and systems using on an effective AI model tailored for HSAT, that can predict or identify sleep arousals using non-brain signal groups, or in other words using signals not obtained from a brain-machine-interface (BMI). Also provided, as embodiments, are methods and systems using on an effective AI model tailored for HSAT, that can predict sleep arousals or arousal-associated events using only non-EEG signal groups. And even further are provided, as further embodiments, methods and systems using on an effective AI model tailored for HSAT, that can predict sleep arousals using only two non-EEG signal groups. And in preferred embodiments, methods and systems are provided that predict sleep arousals or arousal-associated events using respiratory signals without requiring brain signal groups.

A non-invasive method and system are provided for determining an arousal or arousal-associated events of a subject. The method includes (1) obtaining one or more respiratory signals, the one or more respiratory signals being an indicator of a respiratory activity of the subject, (2) extracting features from the one or more respiratory signals, and (3) determining an arousal or an arousal-associated event of the subject based on the extracted features.

A method and system is provided for determining an arousal or an arousal-associated event in a sleep study of a subject, the method comprising: obtaining data from one or more body signals, the one or more body signals being non-brain signals; and determining an arousal or an arousal-associated event of the subject using the data from one or more body signals.

### BRIEF DESCRIPTION OF THE DRAWINGS

FIG. 1 shows an example of a hypopnea event.

FIG. 2 shows an example of a scored apnea followed by a desaturation and an arousal.

FIG. 3 shows examples of scored hypopneas followed by desaturations and arousals.

FIGS. 4a and 4b illustrate an example of respiratory inductance plethysmograph (RIP) belts.

FIG. 5 shows an example of a scored apnea followed by a desaturation and an arousal as determined according to a preferred embodiment.

FIG. 6 shows examples of scored hypopneas followed by desaturations and arousals as determined according to a preferred embodiment.

FIGS. 7A, 7B, and 7C show schematics of a subject sleeping with HSAT sleep study devices.

FIG. 8 illustrates a computing device configured to perform the method of determining arousals based on received data obtained from the subject of the sleep study.

FIG. 9 shows a structure of a single gated recurrent unit (GRU) unit.

FIG. 10 shows a diagram of the neural network.

FIG. 11 shows a flowchart depicting the various exclusion criteria and the final dataset values for validation of the disclosed method.

FIG. 12 shows a visual representation of how test and reference AHI were obtained in the validation of the disclosed method.

FIG. 13 shows a Bland Altman plot for the whole cohort.

FIGS. 14A and 14B show Bland Altman plots of the reference and test AHI for a) males (FIG. 14A) and b) females (FIG. 14B).

FIGS. 15A, 15B, 15C, 15D, 15E, and 15F show Bland Altman plot of the reference and test AHI for all the different age groups: a) 18-25 years (FIG. 15A), b) 26-35 years (FIG. 15B), c) 36-45 years (FIG. 15C), d) 46-55 years (FIG. 15D), e) 56-65 years (FIG. 15E), and f) 65+ years (FIG. 15F).

FIGS. 16A, 16B, and 16C show Bland Altman plot of the reference and test AHI for the different BMI groups: a) Normal (BMI <25) (FIG. 16A), b) Overweight (25≤BMI <30) (FIG. 16B), and c) Obese (BMI ≥30) (FIG. 16C).

FIGS. 17A and 17B show that the majority of data points were centered around the zero difference line for both groups with comorbidity and the group without a known comorbidity.

FIGS. 18A and 18B show a Bland-Altman plot showing agreement for individuals taking medication (FIG. 18A) and individuals not taking medication (FIG. 18B).

FIG. 19 show a flowchart used for defining the medication study population.

FIG. 20 shows an example of how the thoracic RIP signal changes in a period of REM sleep interrupted by an awakening and a period of non-REM sleep.

FIGS. 21A and 21C show the systematic underestimation of Home Apnea Sleep Testing (HSAT) Respiratory Event Index (REI) using current HSAT methods compared to Polysomnography (PSG) Apnea Hypopnea Index (AHI), and 21B and 21D show the significant improvement to accuracy of the Home Apnea Sleep Testing (HSAT) Respiratory Event Index (REI) using an embodiment of the non-brain, BodySleep analysis method as described herein.

FIG. 22 shows a method of obtaining results from a home sleep study (HSS) that are significantly improved to level of accuracy comparable to a PSG study.

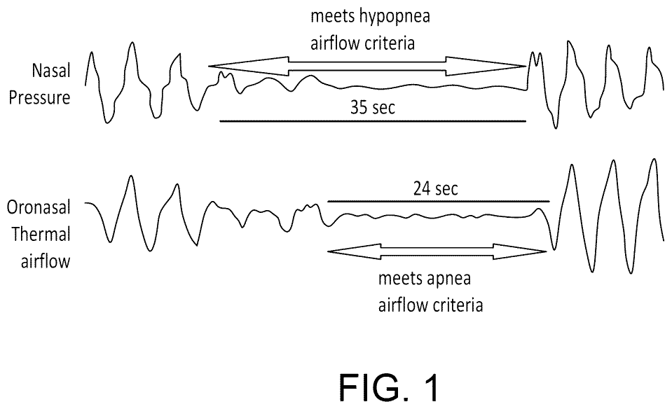

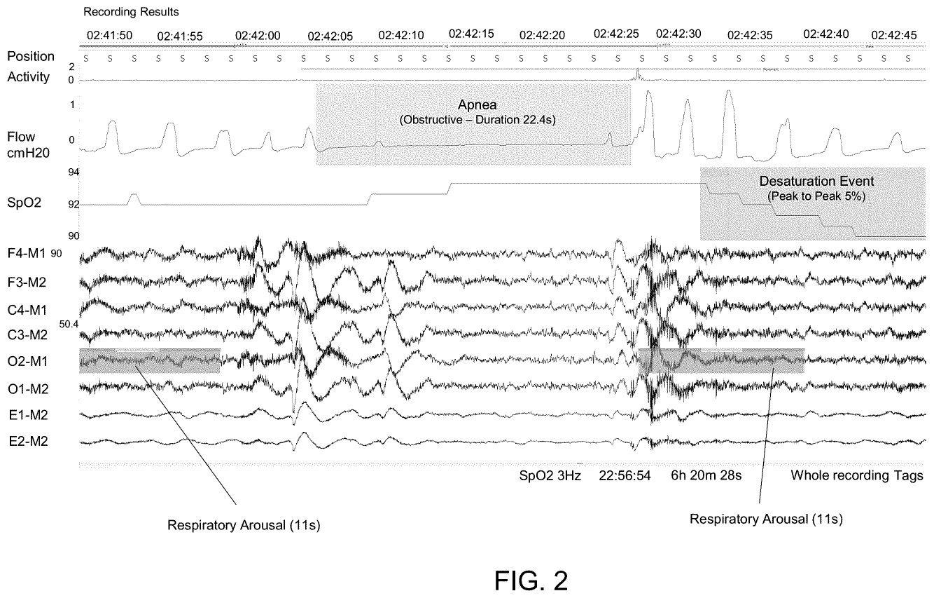

### DETAILED DESCRIPTION OF VARIOUS EMBODIMENTS

As noted above, the inventors of the present application have found it would be highly advantageous to have some prediction or determination of an arousal or an arousal-associated event, such as an arousal-associated hypopnea, during a sleep study of a subject to improve the accuracy of a sleep-related diagnoses. But doing an electroencephalography (EEG) on a patient during a Home Apnea Sleep Testing (HSAT) is often impractical or impossible in a home-type setting, or is too expensive. Preferably, in a home setting, the patient is able to apply to himself the sensors needed for the HSAT, which simply is not practical with EEG.

Additionally, a sleep study including an arousal prediction, determination, or classification based on cardio or heart-related signals or body movement signals are often inaccurate, due to the dependency of cardio or heart-related signals to unrelated factors, such as cardiac condition, blood pressure, medication and other individual specific factors.

What would be highly advantageous is that an arousal or an arousal-associated event determination be performed in what may be termed a “body sleep study”, meaning a sleep study performed without using or at least without requiring features derived directly from the brain, that is without using or at least without requiring features obtained by a brain-machine interface (BMI), that translates neuronal activity of the brain into signals, such as an electroencephalogram (EEG). Further, it would be highly advantageous to perform an arousal or an arousal-associated event determination in a HSAT or sleep study not including EEG without using or at least without requiring features derived from the heart. Further, it would also be preferred that a HSAT or sleep study not including EEG to perform an arousal determination based on measuring one or more features other than body movement signals, as an arousal or an arousal-associated event determination of a sleep study based on body movement signals can be inaccurate. Such a sleep study, based on or using non-brain signals, and even more preferably non-brain and non-cardiac signals would allow the sleep study to be performed with improved certainty and accuracy on patients, and particularly in a home environment, and would greatly reduce the risk of wrong clinical decisions or requiring of a further non-home-based sleep study, such as a general Polysomnography (PSG).

As used herein, an “arousal-associated event” or “arousal-associated events” are events that occur in a relation with or are correlated with an arousal. Such events can occur prior to an arousal, simultaneously with an arousal, or post or after an arousal. An event is associated with an arousal when an arousal causes the associated event, the associated event causes the arousal, or the arousal and the arousal associated event have a mutual cause. Such events can include, but are not limited to: a transition from sleep to awake state, when a transition from sleep to wake is preceded by an arousal or occurs at the same time as an arousal; a transition from NREM2 sleep to NREM1 sleep or a period of time within a transition from NREM2 sleep to NREM1 sleep, for example a 10 second epoch, a 20 second epoch, a 30 second epoch, or up to a 60 second epoch; a transition from REM sleep to NREM1 sleep; when an arousal interrupts stage REM sleep; a period limb movement of sleep (PLMS) or limb movement (LM) associated arousal; an arousal and a limb movement that occur in a periodic limb movement (PLM) series could be considered associated with each other if they occur simultaneously, overlap, or when they occur within a certain period of time of each other, such as <0.1 seconds, <0.25 seconds, <0.5 seconds, <0.75 second, <1.0 seconds, <1.5 seconds, <2.0 seconds, <5.0 seconds, <10.0, <30.0 seconds, <45.0 seconds, <60.0 seconds, <90.0 seconds, or <120.0 seconds, or <180.0 seconds between the end of one event and the onset of the other event; a hypopnea that terminates in an arousal; the arousal starts during or shortly after the hypopnea; a respiratory effort related arousal (RERA); an arousal due to an external stimulus; a reduction in airflow that does not meet the criteria for hypopnea (>=30% reduction in flow) or apnea (>=90% reduction in flow) ends in an arousal; or as described by the AASM Manual for the Scoring of Sleep and Associated Events cited above and incorporated herein by reference, when an arousal interrupts stage N2 sleep; when an arousal interrupts stage R sleep; when an arousal interrupts stage R sleep followed by a low-amplitude, mixed frequency EEG without posterior dominant rhythm and with slow eye movements; or for a hypopnea if the peak signal excursions drop by ≥30% of pre-event baseline, the duration of the ≥30% drop is signal excursion is ≥10 seconds, and there is a ≥3% oxygen desaturation from pre-event baseline; and/or in the case of a respiratory effort related arousal (RERA, if there is a sequence of breaths lasting ≥10 seconds characterized by increasing respiratory effort or by flattening of the inspiratory portion the nasal pressure or PAP device flow waveform leading to an arousal from sleep when the sequence of breaths does not meet criterial for an apnea or hypopnea.

### Body Signals

Preferably, the body signals—that is, the non-brain signals—are obtained by non-invasive means or sensors. As used herein, a method, sensor, or procedure may be described as non-invasive when no break in the skin is created and there is no contact with the mucosa, or skin break, or internal body cavity beyond a natural body orifice. In the context of sleep studies or determining a sleep stage of a subject, the term invasive may be used to describe a measurement that requires a measurement device, sensor, cannula, or instrument that is placed within the body of the subject, either partially or entirely, or a measurement device, sensor, or instrument placed on the subject in a way that interferes with the sleep or the regular ventilation, inspiration, or expiration of the subject. For example, a measuring of esophageal pressure (Pes), which is considered the gold standard in measuring respiratory effort, requires the placement of a catheter or sensor inside the esophagus and is therefore considered an invasive procedure and is not practical for general respiratory measures. Other known output values can be derived from invasive measurements, such as direct or indirect measure of intra thoracic pressure PIT and/or diaphragm and intercostal muscle EMG. as esophageal pressure (Pes) monitoring, epiglottic pressure monitoring (Pepi), chest wall electromyography (CW-EMG), and diaphragm electromyography (di-EMG). Each of these methods suffers from being invasive.

Non-invasive methods to measure breathing movements and respiratory effort may include the use of respiratory effort bands or belts placed around the respiratory region of a subject. The sensor belt may be capable of measuring either changes in the band stretching or the area of the body encircled by the belt when placed around a subject's body. A first belt may be placed around the thorax and second belt may be placed around the abdomen to capture respiratory movements caused by both the diaphragm and the intercostal-muscles. When sensors measuring only the stretching of the belts are used, the resulting signal is a qualitative measure of the respiratory movement. This type of measurement is used, for example, for measurement of sleep disordered breathing and may distinguish between reduced respiration caused by obstruction in the upper airway (obstructive apnea), where there can be considerable respiratory movement measured, or if it is caused by reduced effort (central apnea), where reduction in flow and reduction in the belt movement occur at the same time.

Unlike the stretch-sensitive respiratory effort belts, areal sensitive respiratory effort belts provide detailed information on the actual form, shape and amplitude of the respiration taking place. If the areal changes of both the thorax and abdomen are known, by using a calibration, the continuous respiratory volume can be measured from those signals and therefore the respiratory flow can be derived.

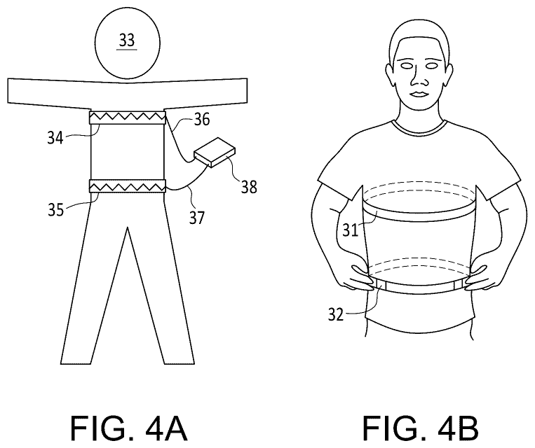

The inventors have developed a method and system for determining arousals or arousal-associated events based on or using breathing features, body activity features, or a combination of breathing and body activity features but excluding or at least not requiring brain features or cardio features. For example, the method may be based on using only the signals from one or more respiratory inductance plethysmography (RIP) belts intended for measuring respiratory movements of the thorax and abdomen. FIGS. 4a and 4b illustrate an example of respiratory inductance plethysmograph (RIP) belts. FIG. 4a shows an example of the wave-shaped conductors in the belts, and FIG. 4b shows the cross-sectional area of each belt, which is proportional to the measured inductance.

### Respiratory Inductive Plethysmography (RIP)

Respiratory Inductive Plethysmography (RIP) is a method to measure respiratory related areal changes. As shown in FIGS. 4a and 4b, in RIP, stretchable belts 31, 32 may contain a conductor 34, 35 that when put on a subject 33, form a conductive loop that creates an inductance that is proportional to the absolute cross sectional area of the body part that is encircled by the loop. When such a belt is placed around the abdomen or thorax, the cross-sectional area is modulated with the respiratory movements and therefore also the inductance of the belt. Conductors 34, 35 may be connected to signal processor 38 by leads 36, 37. Processor 38 may include a memory storage. By measuring the belt inductance, a value is obtained that is modulated directly proportional with the respiratory movements. RIP technology includes therefore an inductance measurement of conductive belts that encircle the thorax and abdomen of a subject. As used herein, a respiratory signal may be obtained by the respiratory signal being received by a processor directly from the RIP belts, by a processor receiving a pre-processed respiratory signal that had originally been obtained from the RIP belts, or a respiratory signal may be obtained by a processor by the processor receiving a respiratory signal that was previously obtained from a subject and stored on a memory storage, either in a raw unprocessed form or in a pre-processed form, and subsequently obtained or received by the processor from the memory storage. The memory storage may be a separate device from the processor, may be hardwired to the processor, or the stored respiratory signal may be transmitted to the processor, for example, over a network or another communications connection (either hardwired, wireless, or a combination of hardwired or wireless) to a computer system that includes the processor. Alternatively, the respiratory signals may be analyzed in real time.

In another embodiment, conductors may be connected to a transmission unit that transmits respiratory signals, for example raw unprocessed respiratory signals, or semi-processed signals, from conductors to processing unit. Respiratory signals or respiratory signal data may be transmitted to the processor by hardwire, wireless, or by other means of signal transmission.

Resonance circuitry may be used for measuring the inductance and inductance change of the belt. In a resonance circuit, an inductance L and capacitance C can be connected together in parallel. With a fully charged capacitor C connected to the inductance L, the signal measured over the circuitry would swing in a damped harmonic oscillation with the following frequency:

$$f = \frac{1}{2 π \sqrt{LC}} , \qquad ( 1 )$$

until the energy of the capacitor is fully lost in the circuit's electrical resistance. By adding to the circuit an inverting amplifier, the oscillation can however be maintained at a frequency close to the resonance frequency. With a known capacitance C, the inductance L can be calculated by measuring the frequency f and thereby an estimation of the cross-sectional area can be derived.

The method for determining arousals or arousal-associated events, such as arousal-associated hypopneas, using breathing signals or breathing signals in combination with other body activity features but excluding or at least not requiring brain features or cardio features may also include using a signal from an activity sensor.

### Other Body Signals

In addition to or in place of RIP signals, embodiments of the method may include using other physiological signals to detect arousals or arousal-associated events. Other physiological signals may be used to confirm or corroborate arousals or arousal-associated events detected by respiratory inductance plethysmography (RIP), or other physiological signals may be used in combination with RIP signals to detect, determine, or predict arousals or arousal-associated events. Moreover, one or more other physiological signals, different than RIP signals, may alone be the basis of and used to determine arousals during a sleep study using a method similar to that relied on for the determination of arousals using RIP signals. For example, an oximetry signal (SpO2 signal) could be used as a lone or only signal from which an arousal or an arousal-associated event is determined. Other signals may include, but are not limited to, accelerometer signals, audio signals, cardiovascular signals, oximetry, non-cardiac electrode potentials, signals indicating body position, video signals, temperature signals, peripheral arterial tone (PAT) measurements pulse, heart rate, heart rate variability, changes in pulse wave amplitude (PWA), changes in pulse transit time (PTT), and other cardiovascular signals, or galvanic skin response (GSR) or combinations thereof to detect arousals.

As described below, the method and system may be based on using a Nox HSAT recorder to record RIP or RIP and body activity signals during the night and then subsequently uploading recorded data signals to a computer after the study is completed. Of course, other HSAT recording devices may be used. Software may be used to derive multiple respiratory parameters from those signals or to derive multiple respiratory parameters activity parameters from those signals, such as respiratory rate, delay between the signals, stability of the respiration and ratio of amplitude between the two belts.

When the parameters have been derived, they may then be fed into a computing system. For example, in a first embodiment the parameters are fed into an artificial neural network computing system that has been trained to predict arousals or arousal-associated events of the subject. An artificial neural network computing system may also be trained to predict the three sleep stages, Wake, REM and NREM, which may be used to plot a simplified hypnogram for the night. The classifier computing system might be different than an artificial neural network. For example, in another embodiment a support vector machine (SVM) method could be used, clustering methods could be used, and other classification methods exist which could be used to classify epochs of similar characteristics into one of several groups. In the first embodiment of the method, an artificial neural network, and preferably a convolutional neural network (CNN) was used. This method can be used on a standard HSAT, does not add any burden to the patient or subject, and may be provided in a fully automated way by the physician.

### Solution of Preferred Embodiments

According to a preferred embodiment, the inventors have developed a method (referred to by the Applicant Nox as “BodySleep2.0” but is also referred to herein simply as “BodySleep”) of detecting sleep and arousals or arousal-associated events using physiological signals other than EEG, or more generally physiological signals other than brain signals or using non-brain signal groups, or using physiological signals that are not obtained from a brain-machine-interface (BMI).

In a preferred embodiment of the method respiratory inductance plethysmography (RIP) or respiratory inductance plethysmography (RIP) in combination with one or more activity signals are used to detect arousals. This is particularly useful to detect arousals following respiratory events, since it has been found that one signature of the termination of a respiratory event are large recovery breaths. Recovery breaths are large breaths following a period of reduced breathing. These breaths typically result in a larger amplitude swing in the measured flow signal than baseline and the breaths flow signals may also have distinct shapes.

Other embodiments of the method may include using other physiological signals to detect arousals or arousal-associated events. Other physiological signals may be used to confirm or corroborate arousals detected by respiratory inductance plethysmography (RIP), or other physiological signals may be used in combination with RIP signals to detect, determine, or predict arousals. Such other signals may include, but are not limited to, accelerometer signals, Peripheral Arterial Tona (PAT) measurements pulse, heart rate, heart rate variability, changes in pulse wave amplitude (PWA), changes in pulse transit time (PTT), and other cardiovascular signals, or galvanic skin response (GSR) to detect arousals.

The methods, systems, and devices proposed herein may be used in combination with those described in Applicant's U.S. patent application Ser. No. 17/026,844, filed at the USPTO on Sep. 21, 2020, and published on Mar. 25, 2021 as US 2021/0085242A1, which describes system and method for determining sleep stages based on non-cardiac body signals (i.e., which has been referred to by the Applicant as the first “BodySleep”), the contents of which are herein incorporated by reference. Additionally, the methods, systems, and devices proposed herein may be used in combination with those described in Applicant's U.S. patent application Ser. No. 17/351,933, filed as the USPTO on Jun. 18, 2021, and published on Dec. 23, 2021 as US 2021/0393211A1, which describes system and method for personalized sleep classifying methods and systems, the contents of which are herein incorporated by reference.

Although not necessary for the implementation of the arousal-determination method disclosed herein, according to an embodiment, non-cardiac signals can be used to detect sleep stages for every predetermined period of the sleep study, for example, preferably every 30 second period or what may termed an epoch. Of course, the resolution of the study, or the length of the periods of the sleep study can be varied. The predetermined periods can be 10 minutes, 5 minutes, 3 minutes, 2 minutes, 60 seconds, 45 seconds, 30 seconds, 20 seconds, or even 10 seconds. When detecting sleep stages every period (for example, 30 second period (epoch)) is classified as one of Wake, NREM, or REM sleep stages. Other embodiments might classify different length periods into sleep stages, and the sleep stages of interest might be different such as Wake, light sleep, deep sleep, REM; or Wake, NREM 1, NREM 2, NREM 3, REM sleep, or any other sleep stages.

When detecting the arousals or arousal-associated events, such as an arousal-associated hypopnea, the method of the present disclosure determines the probability of an arousal or an arousal-associated event occurring at a predetermined interval, such as at every second, and if the probability crosses a certain threshold for a given amount of time an arousal event is scored. The interval for the scoring of arousals may be varied, similar to the epochs for the sleep stage determinations, and may be 10 minutes, 5 minutes, 3 minutes, 2 minutes, 60 seconds, 45 seconds, 30 seconds, 20 seconds, or even 10 seconds, 5 seconds, 3 seconds, 2 seconds, 1 second, or less than 1 second, such as ¾ second, ½ second, ¼ second, or less. Different embodiments may implement this differently. The one second interval may be an arbitrary choice, and how long an interval or period the probability of an arousal has to be above a threshold can also be changed to meet the needs of the sleep study.

In a preferred embodiment, convolutional neural networks (CNN) are deployed, which are a type of artificial neural networks. Other types of artificial neural networks may also be used. The raw recorded RIP and activity signals are input into the CNNs and the events are output. Different types of classifiers can also be used for this purpose and the inputs can be the raw signals or predetermined features of the signals.

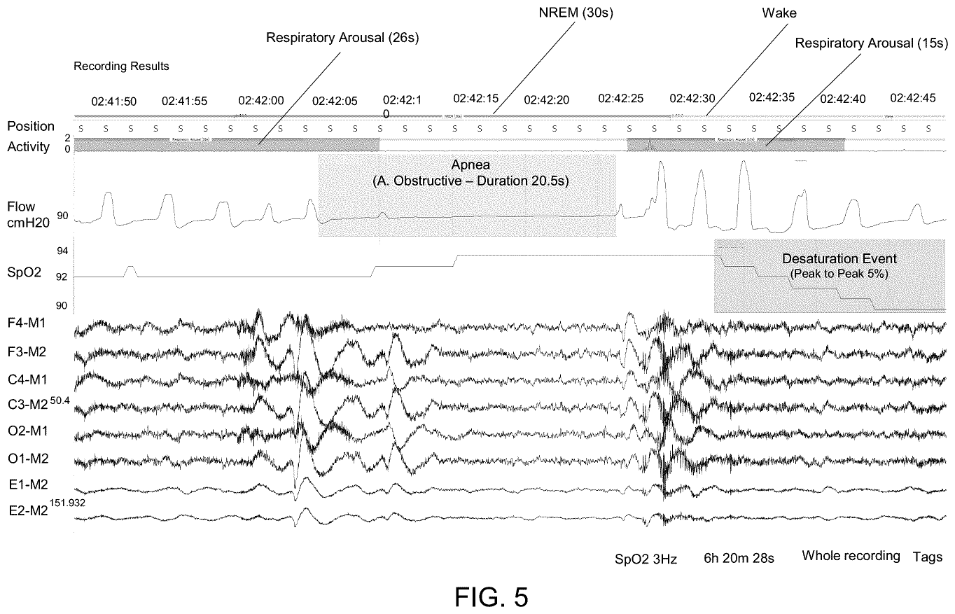

FIG. 5 shows an example of a scored apnea followed by a desaturation and an arousal as determined according to a preferred embodiment. In FIG. 5 the apnea is marked with the orange square on top of the flow signal. The desaturation event is marked by the sea green square on top of the SpO2 signal, and the arousals are the sea green squares on top of the Activity signal. In FIG. 5 the arousals were scored with the current embodiment. FIG. 5 also shows that each of the epochs are classified as NREM, NREM, and Wake sleep stages. This is illustrated below the timeline and above the row of “S” characters. There NREM is painted in sea green and wake in yellow.

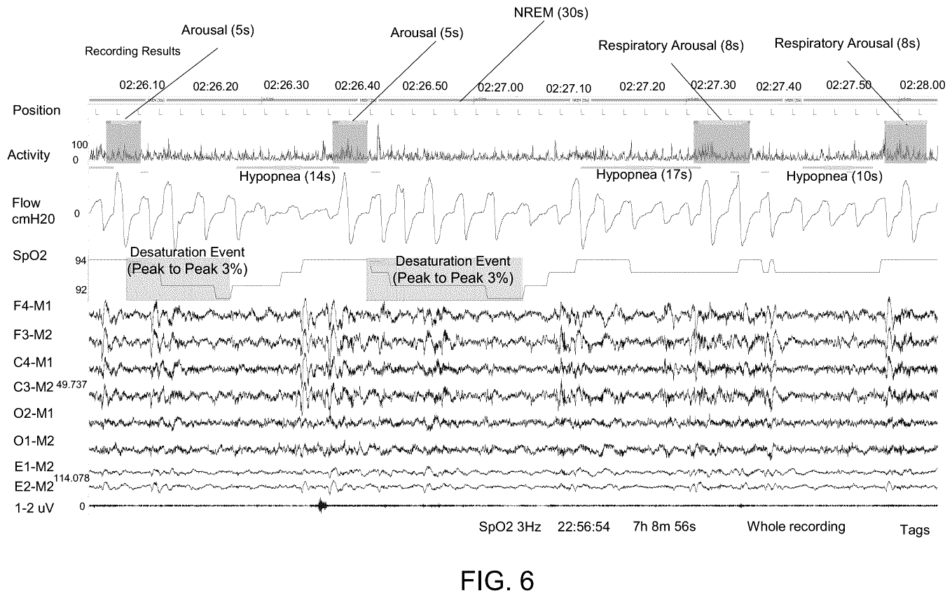

FIG. 6 shows examples of scored hypopneas followed by desaturations and arousals as determined according to a preferred embodiment. The hypopnea events are marked with cyan bars drawn above the flow signal. The reduction in flow during the hypopneas is marked by the reduction in the flow signal amplitude. Desaturation events are labeled and marked with sea green squares on top of the SpO2 signal in the first half of the figure. The arousal events are the sea green squares on top of the Activity signal. FIG. 6 also shows that each of the epochs are classified as NREM sleep stage. This is illustrated below the timeline and above the row of “L” characters. There NREM is painted in sea green.

Determining Respiratory Related Arousal (RERA) As noted above, a Respiratory Related Arousal (RERA) is a respiratory event that does not fulfill the criteria of apneas or hypopneas, that terminates in an arousal. Using the abdomen and thoracic RIP signals as input to a model one can determine changes in airflow. Applying the method of the above-described preferred embodiments to score arousals would allow directly scoring of the respiratory events, without outputting the arousals.

### Predicting Apnea-Hypopnea-Index (AHI)

Using the above-described embodiments, whether using RIP signals, an oximetry signal, or an activity signal, such as an accelerometer signal, either alone or in combination with other non-brain type body signals, one can predict the respiratory events during a sleep study. Based on the described method, a method of outputting the apnea-hypopnea-index (AHI) is also provided. The AHI is the clinical parameter used to determine if a patient is eligible for sleep apnea treatment. To calculate the AHI uses the number of apneas and hypopneas and an estimation of the sleep time. One practice is to use the recording time as an estimate of the sleep time. This results in a parameter called the respiratory event index (REI). Using the body sleep method disclosed herein would allow the determination of the actual sleep time and in that case the calculation of an AHI. An index called the respiratory disturbance index (RDI) is the number of apneas, hypopneas, and RERAs per hour of sleep.

If the AHI is predicted, then directly predicting the AHI severity classification can also be performed. Traditionally there are cutoffs of the AHI that signify if a patient is healthy AHI <5 events/hour, AHI >=5 and AHI <15 a patient may be eligible for treatment depending on other symptoms. A patient with AHI >=15 is eligible for sleep apnea treatment.

So the preferred methods disclosed herein may be used to directly predict the AHI classification, the AHI, or the respiratory events.

### Performance of Preferred Embodiments in First Validation

According to the preferred embodiment, the method was first validated on 90 sleep recordings from a sleep clinic in the United States. Below are the results of the validation.

### Sleep Stage Classification in First Validation

As shown in Table 1-1 below, in a first validation process, two embodiments of the Nox BodySleep were validated. A first embodiment used the RIP, Activity, and signals from the pulse oximeter (Pulse Wave and SpO2) as inputs. A second embodiment used only the RIP and Activity signals as inputs. The outputs are labels for each 30 second period (epoch) in the sleep study. The performance of the embodiment was compared with the gold standard, manually scored polysomnography (PSG) sleep studies. A confusion matrix was constructed showing the epoch level agreement in the classification and the Sensitivity, Specificity, Accuracy, Matthews Correlation Coefficient (MCC), and F1 scores calculated.

TABLE 1-1

| RIP + | | | | | |
|---|---|---|---|---|---|
| Activity + | | BodySleep (Sleep stage + arousal model) | | | Sum of |
| Oximeter | | Wake (0) | REM (1) | NREM (2) | epochs |
| PSG | Wake (0) | 12,665 | 166 | 2,794 | 15,625 |
| | REM (1) | 183 | 6,442 | 1,537 | 8,162 |
| | NREM (2) | 1,742 | 1,431 | 43,481 | 46,654 |
| | | | | | 70,441 |

| | | | Class | | | Accuracy |
|---|---|---|---|---|---|---|
| | Sensitivity | Specificity | accuracy | MCC | F1 | 0.89 |
| Wake (0) | 0.81 | 0.96 | 0.93 | 0.82 | 0.84 | |
| REM (1) | 0.79 | 0.97 | 0.95 | 0.78 | 0.80 | |
| NREM (2) | 0.93 | 0.82 | 0.89 | 0.82 | 0.92 | |

| RIP + | | BodySleep (Sleep stage + arousal model) | | | Sum of |
|---|---|---|---|---|---|
| Activity | | Wake (0) | REM (1) | NREM (2) | epochs |
| PSG | Wake (0) | 12,693 | 176 | 2,756 | 15,625 |
| | REM (1) | 178 | 6,378 | 1,606 | 8,162 |
| | NREM (2) | 1,505 | 1,204 | 43,945 | 46,654 |
| | | | | | 70,441 |

| | | | Specificity | Class | | |
|---|---|---|---|---|---|---|
| | | Sensitivity | Class | accuracy | | Accuracy |
| | Sensitivity | Specificity | accuracy | MCC | F1 | 0.89 |
| Wake (0) | 0.81 | 0.97 | 0.93 | 0.83 | 0.85 | |
| REM (1) | 0.78 | 0.98 | 0.96 | 0.79 | 0.80 | |
| NREM (2) | 0.94 | 0.82 | 0.90 | 0.82 | 0.93 | |

As shown in Table 1-2 below, the performance of the arousal scoring was validated on data from a sleep clinic in the United States. The performance of the arousal scoring was done by calculating the Sensitivity, Specificity, and Accuracy for the presence or absence of an arousal event within a 30 second epoch. The gold standard manual scoring of PSG sleep studies was used as the reference. The performance was validated for different detection thresholds of the arousal scoring. The performance of the method of this preferred embodiment was compared to the performance of the Noxturnal™ PSG arousal scoring model, which is a released medical device in Europe.

TABLE 1-2

| RIP + | | | |
|---|---|---|---|
| Activity + | | | |
| Oximeter | Arousals (Epoch level agreement) | | |
| Threshold | Sensitivity | Specificity | Accuracy |
| 1 | 0 | 100 | 78 |
| 0.09 | 41 | 96 | 84 |
| 0.04 | 57 | 91 | 84 |
| 0.03 | 71 | 80 | 78 |
| PSG model | 67 | 91 | 86 |
| RIP + | | | |
| Activity | Arousals (Epoch level agreement) | | |
| Threshold | Sensitivity | Specificity | Accuracy |
| 0.09 | 41 | 95 | 83 |
| 0.05 | 50 | 92 | 82 |
| 0.04 | 55 | 89 | 81 |
| 0.035 | 60 | 85 | 80 |
| 0.03 | 78 | 65 | 68 |
| PSG model | 67 | 91 | 86 |

Table 1-3 shows the arousal scoring was used to determine if a reduction in airflow during sleep is considered a Hypopnea. The impact of the arousal scoring from the this preferred embodiment on hypopnea scoring was validated

TABLE 1-3

| RIP + | | | |
|---|---|---|---|
| Activity + | | | |
| Oximeter | Arousals (Epoch level agreement) | | |
| Threshold | Sensitivity | Specificity | Accuracy |
| 1 | 62 | 100 | 93 |
| 0.09 | 78 | 99 | 95 |
| 0.04 | 84 | 97 | 95 |
| 0.03 | 89 | 94 | 93 |
| PSG model | 89 | 98 | 96 |
| RIP + | | | |
| Activity | Arousals (Epoch level agreement) | | |
| Threshold | Sensitivity | Specificity | Accuracy |
| 0.09 | 77 | 99 | 95 |
| 0.05 | 81 | 97 | 94 |
| 0.04 | 83 | 96 | 94 |
| 0.035 | 85 | 95 | 93 |
| 0.03 | 91 | 90 | 90 |
| PSG model | 89 | 98 | 96 |

As shown in Tables 1-4 and 1-5, below, the hypopnea scoring was used to calculate the Apnea-Hypopnea Index (AHI) which is an index of how many apneas and hypopneas occur during each hour of sleep (events/hour). Sleep apnea severity is classified using the AHI index. The impact of the arousal scoring on the sleep apnea severity was investigated for the AHI cutoff values of AHI >=5 events/hour (Table 1-4), and AHI >=15 events/hour (Table 1-5). These cutoff values were chosen since they represent the clinical cutoff values used by the Centers for Medicare and Medicaid in the United States to determine if a patient is eligible for sleep apnea treatment.

TABLE 1-4

| RIP + | | | |
|---|---|---|---|
| Activity + | | | |
| Oximeter | AHI >= 5 | | |
| Threshold | Sensitivity | Specificity | Accuracy |
| 1 | 68 | 100 | 75 |
| 0.09 | 84 | 100 | 88 |
| 0.04 | 91 | 97 | 92 |
| 0.03 | 97 | 97 | 97 |
| PSG model | 96 | 95 | 96 |
| RIP + | | | |
| Activity | AHI >= 5 | | |
| Threshold | Sensitivity | Specificity | Accuracy |
| 0.09 | 80 | 100 | 84 |
| 0.05 | 87 | 100 | 89 |
| 0.04 | 90 | 100 | 92 |
| 0.035 | 93 | 100 | 95 |
| 0.03 | 100 | 92 | 98 |
| PSG model | 96 | 95 | 96 |

TABLE 1-5

| RIP + | | | |
|---|---|---|---|
| Activity + | | | |
| Oximeter | AHI >= 15 | | |
| Threshold | Sensitivity | Specificity | Accuracy |
| 1 | 54 | 100 | 80 |
| 0.09 | 74 | 98 | 87 |
| 0.04 | 87 | 98 | 93 |
| 0.03 | 94 | 95 | 95 |
| PSG model | 96 | 96 | 96 |
| RIP + | | | |
| Activity | AHI >= 15 | | |
| Threshold | Sensitivity | Specificity | Accuracy |
| 0.09 | 70 | 100 | 87 |
| 0.05 | 83 | 97 | 91 |
| 0.04 | 85 | 97 | 92 |
| 0.035 | 89 | 97 | 94 |
| 0.03 | 95 | 89 | 92 |
| PSG model | 96 | 96 | 96 |

Further, the results show that the method of this preferred embodiment improves the clinical outcomes of patients who undergo HSAT sleep studies by improving the sensitivity and accuracy of the sleep apnea diagnosis. This is therefore significant as it indicates that an HSAT sleep study with improved sensitivity and accuracy of the apnea diagnosis may be provided according to the disclosed methods and systems herein without requiring a pulse oximeter, which is a relatively expensive device, adds complexity to the sleep study, is prone to failure, and decreases comfort.

As shown in Table 1-6 below, in a different embodiment only the thoracic and abdominal RIP signals (RIP Only) are used to predict the sleep stages and arousals. A third embodiment uses only the activity signals (Activity Only) to predict the sleep stages and arousals. The results again show that the method of this preferred embodiment with only the thoracic and abdominal RIP signals (RIP Only) improves the clinical outcomes of patients who undergo HSAT sleep studies by improving the sensitivity and accuracy of the sleep apnea diagnosis.

TABLE 1-6

| | | | SRIP | SRIP + | Activity |
|---|---|---|---|---|---|
| | | | Only | Activity | Only |
| Sleep | Wake | Precision | 0.888 | 0.869 | 0.861 |
| | | Recall | 0.817 | 0.834 | 0.664 |
| | | f1 score | 0.851 | 0.851 | 0.75 |
| | REM | Precision | 0.818 | 0.805 | 0.761 |
| | | Recall | 0.815 | 0.807 | 0.627 |
| | | f1 score | 0.816 | 0.805 | 0.687 |
| | NREM | Precision | 0.918 | 0.921 | 0.849 |
| | | Recall | 0.943 | 0.933 | 0.94 |
| | | f1 score | 0.93 | 0.927 | 0.892 |
| | | Accuracy | 0.9 | 0.896 | 0.842 |
| | | Cohen kappa | 0.796 | 0.791 | 0.661 |
| | | f1-score weighted | 0.899 | 0.896 | 0.837 |
| | | f1-macro | 0.866 | 0.861 | 0.776 |
| Arousals | | Arousal Precision (1 s) | 0.538 | 0.539 | 0.407 |
| | | Arousal Recall (1 s) | 0.318 | 0.313 | 0.312 |
| | | Arousal f1-score | 0.4 | 0.396 | 0.353 |
| | | Non-arousal | 0.97 | 0.97 | 0.97 |
| | | Precision (1 s) | | | |
| | | Non-arousal | 0.988 | 0.988 | 0.98 |
| | | recall (1 s) | | | |
| | | Non-arousal f1-score | 0.979 | 0.979 | 0.975 |
| | | Accuracy | 0.9597 | 0.9597 | 0.952 |
| | | Cohen kappa | 0.38 | 0.377 | 0.329 |

Further Tables 1-7 and 1-8 below show a further validation of show that using the preferred methods and systems described herein, very accurate results for determining arousals and arousal-associated events in over 2000 sleed studies can be provided using only RIP belt signals without an activity signal. The performance of the arousal scoring was done by calculating the Sensitivity, Specificity, and Accuracy for the presence or absence of an arousal event within a 30 second epoch. The gold standard manual scoring of PSG sleep studies was used as the reference. The performance was validated for different detection thresholds of the arousal scoring. The performance of the method of this preferred embodiment was compared to the performance of the Noxturnal™ PSG arousal scoring model. These results show that the method of this preferred embodiment improves the clinical outcomes of patients who undergo HSAT sleep studies that are based only on RIP signals (wherein no activity signal and no pulse oximeter signal is required or used) by improving the sensitivity and accuracy of the sleep apnea diagnosis. This is therefore significant as it indicates that an HSAT sleep study with improved sensitivity and accuracy of the apnea diagnosis may be provided according to the disclosed methods and systems herein without requiring an activity signal such as an accelerometer or a pulse oximeter, which is a relatively expensive device, adds complexity to the sleep study, is prone to failure, and decreases comfort.

TABLE 1-7
RIP belts only(BodySleep) vs PSG manually
scored (over 2000 studies)

| | mean [95% CI] | mean [95% CI] | mean [95% CI] |
|---|---|---|---|
| | Sensitivity | Specificity | Class Accuracy |
| Wake | 0.81 [0.80, 0.81] | 0.94 [0.94, 0.95] | 0.91 [0.91, 0.92] |
| REM | 0.77 [0.76, 0.78] | 0.98 [0.98, 0.98] | 0.95 [0.95, 0.96] |
| NREM | 0.83 [0.83, 0.84] | 0.91 [0.91, 0.92] | 0.92 [0.91, 0.92] |
| Arousals | 0.73 [0.72, 0.74] | 0.96 [0.96, 0.96] | 0.96 [0,96, 0.96] |

TABLE 1-8
BodySleep (RIP belts only) vs PSG manually scored

| | mean [95% CI] | mean [95% CI] | mean [95% CI] |
|---|---|---|---|
| | Sensitivity | Specificity | Class Accuracy |
| Wake | 0.81 [0.80, 0.81] | 0.94 [0.94, 0.95] | 0.91 [0.91, 0.92] |
| REM | 0.77 [0.76, 0.78] | 0.98 [0.98, 0.98] | 0.95 [0.95, 0.96] |
| NREM | 0.83 [0.83, 0.84] | 0.91 [0.91, 0.92] | 0.92 [0.91, 0.92] |
| Arousals | 0.73 [0.72, 0.74] | 0.96 [0.96, 0.96] | 0.96 [0.96, 0.96] |

### Further Performance and Validation

According to further embodiments, a deep learning model was developed to predict arousals, using only respiratory inductance plethysmography (RIP) and activity signal groups. The deep learning model performs a prediction for each recorded second (1-second intervals) and aggregates those results to score arousal events. To train and validate the model, a total of 2216 manually scored PSG sleep recordings were employed from various sleep centers in five countries. The model's robustness and accuracy was tested, using recordings from a separate sleep center that was not included in training or validation. Additionally, we ensured that the recordings covered all categorical severities of sleep apneas; i.e., normal, mild, moderate, and severe.

In this performance evaluation, compared with manual arousal scoring, using epoch-level agreement, the model exhibited a sensitivity, specificity, and accuracy of 62%, 86%, and 81%, respectively. The difference in AHI was investigated when using the model's arousals or no arousals. For AHI>=5, the sensitivity, specificity, and accuracy was 95%, 100%, and 96%, respectively, with arousals, but 68%, 100%, 75% without. Similarly, for AHI>=15, the metrics were 95%, 100%, and 96%, respectively, with arousals, but 54%, 100%, 80%, without. Moreover, for hypopneas, the metrics were 86%, 96%, 94%, respectively, with arousals, a 24% increase in sensitivity compared with not using arousals.

The embodiments provided herein show that respiratory disturbance related arousals can be detected from the RIP belts. The reason for this is that the breathing in the pre-apnea period is too shallow to ventilate the CO2 released in the blood, causing acidification in the blood, increasing respiratory effort and finally causing arousal with large recovery breaths to neutralize the blood gassing of CO2. This effect causes that the recovery breaths can be detected from the RIP bands.

However, further research and modeling using the above-noted data by the inventors has shown that not only does the arousal detection method detect those respiratory disturbance related arousals correctly, but also correctly predicts other arousals as well. This was entirely unexpected and surprising to the inventors. This is confirmed by what has been called “respiratory response to arousals” occurs that is somehow linked to change of control over the respiration between sleep and wake. This can be determined also in the methods disclosed herein as it shows up in the belt signals as well based on the BodySleep2 validation results and may increase the opportunities of usage dramatically.

In sum, the detection model performed well, showing that a HSAT without a brain-based signal can be sufficient in order to predict arousals effectively. Additionally, the inventors' findings show that using the predicted arousals improve the scoring of hypopneas and AHI and that indeed an EEG or brain-based signal is not necessary for an accurate determination of arousals during a sleep study. Further, as described herein, the inventors have developed a method and system that accurately detects respiratory and non-respiratory associated arousals by processing respiratory signals, and particularly in a preferred embodiment, RIP signals. This is achieved by using an artificial neural network (ANN) based analysis that integrates information from both the effect of the change of respiratory control from autonomic to somatic during arousal and the amplitude response at the end of respiratory disorder event. Using this method and a corresponding system, the accuracy for apnea hypopnea index (AHI) analysis from HSAT study is on par and comparable with PSG results, even if only the RIP signals are being used for the AHI diagnoses.

It is further noted that the detection of arousals and arousal-associated events based on a RIP based signals determine or are used to measure a change from automatic to somatic respiratory control, causing respiratory response to arousal. While recovery breaths are a consequence of arousal caused by apena, the respiratory response to arousal in unrelated of the respiratory condition. This effect in the respiratory signal obtained from the RIP signal and used in the arousal and aroussal-associated events as described herein is a “more direct” measure of the arousal than the recovery breath. This is for example the basis for why the method works for PLMS arousals as described below, in that in these cases there is no associated recovery breath. Based on this combinational method of detecting respiratory response to arousal and recovery breaths, the aroussal and arousal-associated events detection methods described herein elevate the accuracy of respiratory analysis of arousals from being considered unusable without EEG reference, according to AASM, to provide accuracy on par with PSG scored arousals. Further, the disclosed method and system provides an effective way for detection for arousals and aroussal-associated events that are not apnea or hypopnea based nor based on sleep disordered breathing, but can be based on non-respiratory events such as PLMS or even external stimuli that arouse the subject.

### Further System and Device Embodiments

Although the subject matter of this disclosure has been described in language specific to structural features and/or methodological acts, it is to be understood that the subject matter defined in the appended claims is not necessarily limited to the described features or acts described above, or the order of the acts described above. Rather, the described features and acts are disclosed as example forms of implementing the claims.

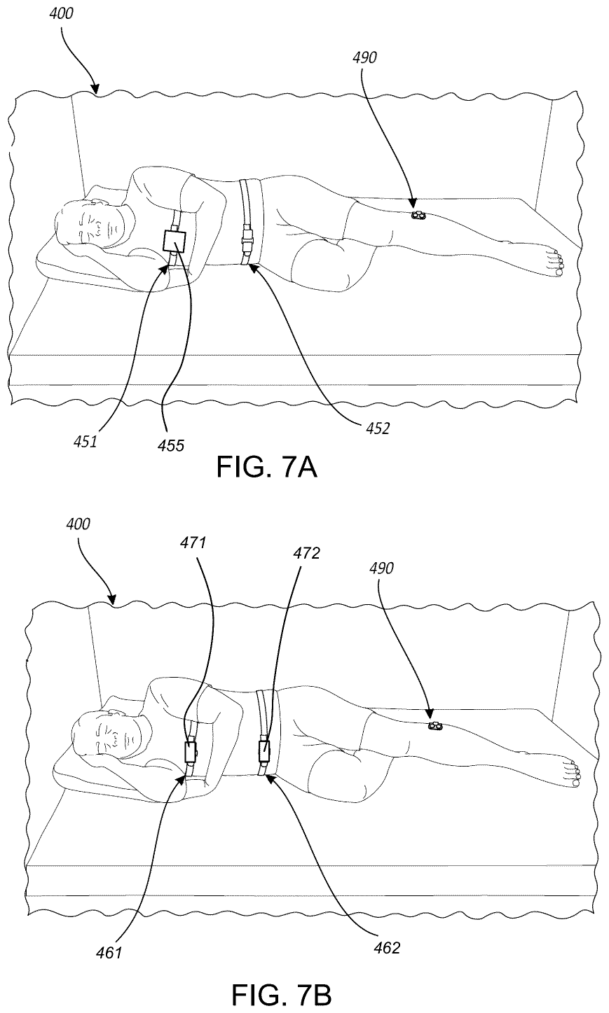

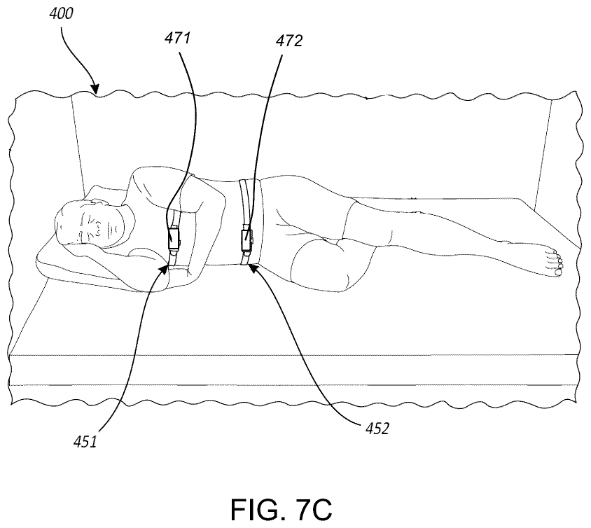

As described above, Respiratory Inductive Plethysmography (RIP) is a method to measure respiratory related areal changes based on stretchable belts 31, 32. FIGS. 7A, 7B, and 7C show schematics of a subject 400 sleeping with HSAT sleep study devices. The devices worn by the subject in such as study may include respiratory inductance plethysmography (RIP) belts 451, 452, arranged about the thoracic region and an abdominal region of the subject, respectively. The RIP belts 451 and 452 may transmit, e.g., wireless or through a wired connection, to a recording device 455 which obtains and stores the data of the RIP signals through the study. As shown in FIG. 7A, a further sensor 490, such as an accelerometer or other activity sensor may be applied to a limb or body portion of the subject, such as on the leg. Data obtained by the further sensor 490, such as activity, motion, or accelerometer data can also be transmitted to and recorded by recording device 455. These sensors and devices may be provided to the subject in such a way that the subject can easily and consistently apply the sensors and devices himself (or herself). Belts 451 and 452 may be disposable devices that would be disposed of by the subject after the sleep study is performed. The belts 451,452 may be configured to be worn for multiple nights, after which the belts can be disposed. Further sensor 490 may also be a disposable device. Recording device 455 need not necessarily be attached to belt 451. Recording device 455 may be returned to the home sleep study administrator and the RIP sleep data and the further sensor data obtained and stored therein may be downloaded or otherwise retrieved by the computer of the sleep study administrator. Alternatively, the recording device 455 may further include a memory and a memory storage, and may be provided with a mobile application to interact with and control the RIP belts 451, 452, and to receive data obtained by the belts and the further sensor. The recording device may further be configured to transmit the data in real time, or at the conclusion of the sleep study, or at a later time, through a network or otherwise wireless connection or over the internet, to a data receiving device of the sleep study administrator.

As shown in FIG. 7B, separate recording devices 471, 472 may be provided to record the signals of the thoracic RIP belt 451 and the abdomen RIP belt 451, respectively. Accordingly, similar to recording device 455, each separate recording device 471, 472 may be configured to record the data obtained from the respectively RIP belt 451,452 and may be either delivered physically or mailed back to the sleep study administrator. Alternatively, the recording devices 471,472 may be configured to transmit the data in real time, or at the conclusion of the sleep study, or at a later time, through a network or otherwise wireless connection or over the internet, to a data receiving device of the sleep study administrator. One or both of recording devices 471,472 may also receive and record and/or transmit data obtained from the signal of the further sensor 490.

Recording device 455 or separate recording devices 471,472 may be power the RIP belts and may be rechargeable by the subject or patient. Belts 451,452 may be activated when the RIP belts are snapped around the patient or a seal is removed from the belts, and are deactivated when unsnapped or otherwise removed from the subject.

Lastly, in the embodiment of FIG. 7C, the home sleep study is administered to the subject without a further sensor 490, but with RIP belts 451, 452 only.

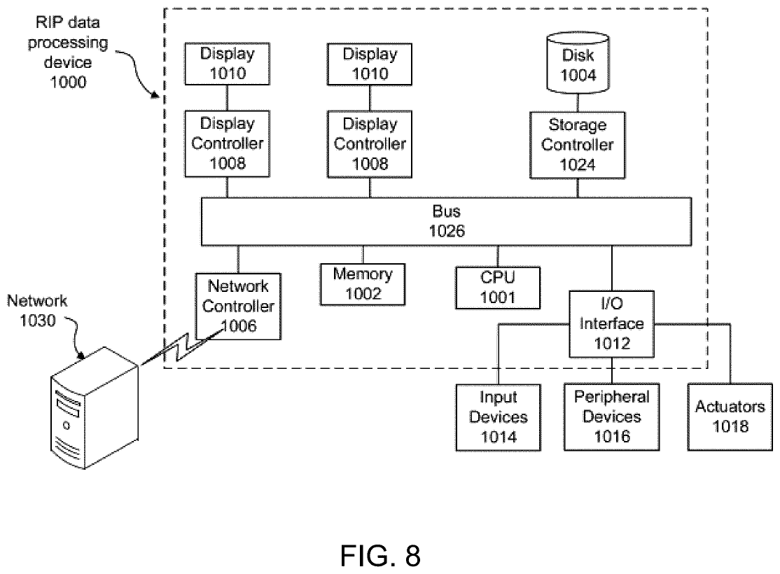

FIG. 8 illustrates a computing device 1000 configured to perform the method of determining arousals of the subject during the sleep study and process the body-signal data (e.g., RIP data). The device 1000 can perform some or all of the steps discussed above. The device 1000 may perform arousal determination method using a utility application, background daemon, or component of an operating system, or combination thereof, executing in conjunction with CPU 1001 and an operating system such as Microsoft Windows 7, UNIX, Solaris, LINUX, Apple MAC-OS and other systems known to those skilled in the art.

CPU 1001 may be a Xenon or Core processor from Intel of America or an Opteron processor from AMD of America, or may be other processor types that would be recognized by one of ordinary skill in the art. Alternatively, the CPU 1001 may be implemented on an FPGA, ASIC, PLD or using discrete logic circuits, as one of ordinary skill in the art would recognize. Further, CPU 1001 may be implemented as multiple processors cooperatively working in parallel to perform the instructions of the inventive processes described above.

The device 1000 in FIG. 8 also includes a network controller 1006, such as an Intel Ethernet PRO network interface card from Intel Corporation of America, for interfacing with a network 1030. The network 1030 can be a public network, such as the Internet, or a private network such as an LAN or WAN network, or any combination thereof and can also include PSTN or ISDN sub-networks. The network 1030 can also be wired, such as an Ethernet network, or can be wireless such as a cellular network including EDGE, 3G and 4G wireless cellular systems. The network 1030 can also be Wi-Fi, Bluetooth, or any other wireless form of a communication that is known.

The device 1000 further includes a display controller 1008 for interfacing with a display 1010. A general purpose I/O interface 1012 interfaces with input devices 1014 as well as peripheral devices 1016. The general purpose I/O interface also can connect to a variety of actuators 1018. The input devices 1014 can include the various sensors, although additional sensors are not necessary for the system. The input devices 1014 may include an interface to receive data from a recording device 455 in FIG. 7A, for example.

A sound controller 1020 may also be provided in the device 1000 to interface with speakers/microphone 1022 thereby providing sounds and/or music.

A general purpose storage controller 1024 connects the storage medium disk 1004 with a communication bus 1026, which may be an ISA, EISA, VESA, PCI, or similar, for interconnecting all of the components of the device 1000. Descriptions of general features and functionality of the display 1010, input devices 1014 (e.g., a keyboard and/or mouse), as well as the display controller 1008, storage controller 1024, network controller 1006, sound controller 1020, and general purpose I/O interface 1012 are omitted herein for brevity as these features are known.

Instructions for the performance of the arousal determination method can be stored on computer storage media and performed using a computation/logic circuitry. For example, instructions for the arousal determining method may be performed on a central processing unit (CPU). Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the disclosure.

Transmission media can include a network and/or data links which can be used to carry program code in the form of computer-executable instructions or data structures, and which can be accessed by a general-purpose or special-purpose computer system. A “network” may be defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions may comprise, for example, instructions and data which, when executed by one or more processors, cause a general-purpose computer system, special-purpose computer system, or special-purpose processing device to perform a certain function or group of functions. Computer-executable instructions may be, for example, binaries, intermediate format instructions such as assembly language, or even source code.

The disclosure of the present application may be practiced in network computing environments with many types of computer system configurations, including, but not limited to, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

The disclosure of the present application may also be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Some embodiments, such as a cloud-computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

### Further Models, Classifiers, and Artificial Neural Networks

### Feature Extraction

The determination of arousal may be based on or include inputting the raw non-brain signal into the neural network as described above. Or alternatively, determination of arousal may be a two-part problem with the first step in the process being the extraction of features from the raw recordings. In an embodiment, a feature extractor was written in Python 3.5.5 to perform this task. The extractor may rely on NumPy and/or SciPy. The output of the feature extractor is a comma-separated values (CSV) file where the rows represent each epoch and the columns contains the features.

In a first embodiment, the signals used are those derived from the abdomen and thorax RIP belts. These include the Abdomen Volume, Thorax Volume, RIPSum, RIPFlow, Phase, and RespRate signals. Additionally, an activity signal from an accelerometer was used. All the features were calculated over a 60, 30, 20, 10, 5, or 1 second interval.

As used herein, Abdomen Volume and Thorax Volume are the RIP signals recorded during the sleep study. The signals may be recorded using the respiratory inductance plethysmography (RIP) bands placed around or on the thorax and abdomen of the subject under study. The RIP signals represent volume in the abdomen and thorax during breathing.

RIPSum is a signal created by adding the samples of Abdomen Volume and Thorax Volume signals. The RIPSum signal is a time series signal of the same number of samples and duration in time as the Abdomen Volume and Thorax Volume signals.

RIPFlow is the time derivative of the RIPSum signal. The RIPSum signal represents volume and the time derivative represents changes in volume which is flow.

Phase is a signal that represents the apparent time delay between the recorded Abdomen and Thoracic volume signals. During normal unobstructed breathing the Abdomen and Thorax move together out and in during inhalation and exhalation. When the upper airway becomes partially obstructed the Abdomen and Thorax start to move out of phase, where either the Abdomen or the Thorax will start expanding while pulling the other back. During complete obstruction of the upper airway the Abdomen and Thorax will start moving completely out of phase, whereas one moves out the other one is pulled inwards. In this case the Phase is 180 degrees, measuring the phase difference between the two signals.

RespRate represents the respiratory rate of the subject under study. The respiratory rate is a measure of the number of breaths per minute and is derived from the Abdomen Volume and Thorax Volume signals.

The feature extractor and the features extracted by the feature extractor are explained herein below. The main points in the description of feature extractor and the features extracted by the feature extractor are:
It works on the recorded signals of Abdomen RIP, Thorax RIP, and accelerometers.
It works on signals derived from the above-mentioned recorded signals. These are the RIPSum, RIPFlow, Phase, RespRate, and Activity.
It splits the signals into 30 second epochs which are used to calculate the features.
Is may be implemented in Python using NumPy and SciPy. This is not an essential feature of the method, just how it was done in in an embodiment.
It outputs results in a CSV file. This is not an essential feature of the method, just how it was done in an embodiment.

The respiration features are calculated from the RIPSum, RIPFlow and RespRate signals. The features calculated were designed to give information about changes in the respiratory rate with various methods.

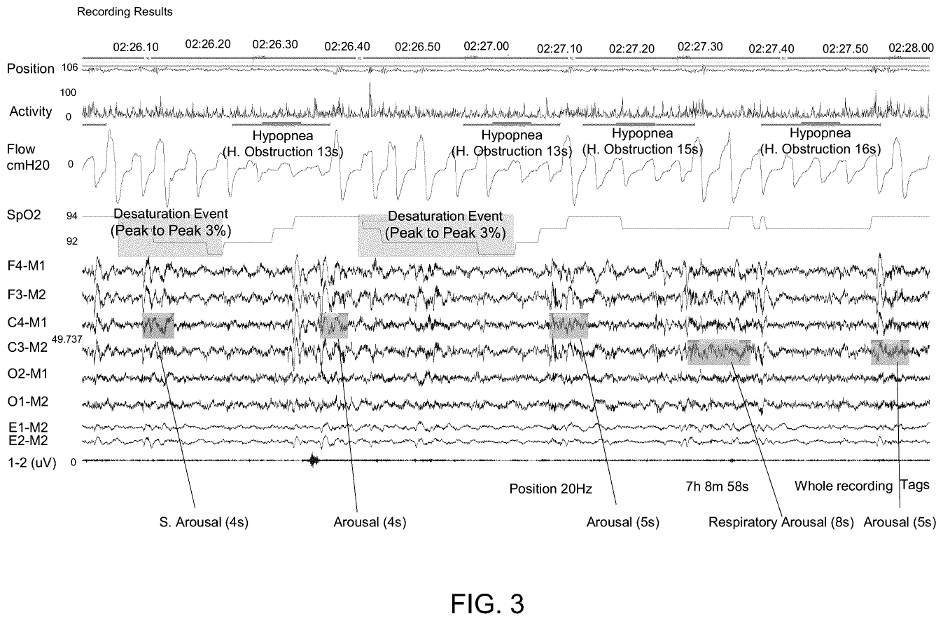

The first harmonic and DC ratio is used to estimate respiratory rate variability. The first harmonic and the DC component are found in the frequency spectrum of a flow signal. For this classifier the RIPFlow was used but some preprocessing required. Such preprocessing included before taking the Fourier transform of the signal, all positive values are made 0, which results in the signal being more periodic as the exhalation is more regular. This can be seen in FIG. 3.

The fast Fourier transform is applied on the resulting signal and the DC component and the first harmonic peak are located. The DC component is defined as the magnitude at 0 Hz and the first harmonic peak is the largest peak of the frequency spectrum after the DC ratio.

The respiratory rate variability with this method may be defined as:

$$RRv = ( 1 \; 00 - \frac{{H}_{1}}{D C} ) \%$$

Where H₁ is the magnitude of the first harmonic peak and DC is the magnitude of the DC component. It has been showed that the RRv is larger in wake and that this size gets smaller as the sleep gets deeper but is larger again in REM sleep. The feature implemented in the final version is just the first harmonic to DC ratio but not the RRv value, since after normalization these values would still be the same.

There may be 4 features that are extracted from the respiratory rate. These features are calculated using mean, standard deviation and difference between epochs. The RespRate signal is used for these calculations. The mean and standard deviation of the respiratory rate is calculated for each epoch. The root means square successive difference (RMSSD) is calculated with

$$RMS SD = \sqrt{\frac{1}{N - 1} {\sum }_{i = 1}^{N - 1} {( {RR}_{i} - {RR}_{i - 1} )}^{2}} \qquad 3.2$$

The difference mean ratio is then calculated as the ratio of the mean respiratory rate of the current epoch and the previous epoch.

The breath-by-breath features are based on features which are calculated for each breath. The final features are then calculated by taking the mean, median or standard deviation of the breath features for each epoch. The breaths may be located by running a breath-by-breath analysis on the RIPsum signal of the whole recording to identify all the breaths. The breaths may then be divided between the 30 s epochs, with breaths that overlap two epochs being placed in the epoch that contains the end of the exhalation of the breath. The signals used for the feature calculations are the RIPsum, RIPflow, Abdomen Volume and Thorax Volume.

In a second embodiment, the breath-by-breath analysis may be based on a start of inhalation being marked as the start of a breath and the end of exhalation being marked as the end of a breath. By adding the correctly calibrated abdomen and thorax RIP signal (as, for example, described in U.S. patent application Ser. No. 14/535,093, filed Nov. 6, 2014, and published as US 2015/0126879; U.S. patent application Ser. No. 15/680,910, filed Aug. 18, 2017, and published as US 2018/0049678; and U.S. patent application Ser. No. 16/126,689, filed Sep. 10, 2018, and published as US 2019/0274586, each of which is incorporated herein by reference in their entirety), calculating a time derivative of the resulting calibrated RIP volume signal results in a flow signal representing breathing airflow. The start of inhalation can be determined by finding points in time where the flow signal crosses a zero value from having negative values to having positive values. The end of exhalation can be determined by finding points in time where the flow signal crosses a zero value from having negative values to having positive values.

What is meant by this is that when the RIP flow signal has a positive value air is flowing into the body, inhalation, and when the RIP flow signal has a negative value air if slowing out of the body, exhalation.

This method of detecting inhalation and exhalation may be simple. But at the same time, for all practical purposes it is not bad and is widely used. It may be noted that high frequency noise in the signal may cause the signal to oscillate, causing multiple zero crossings in periods where the flow rate is low. But to this it can be answered that normal breathing frequency is around 0.3 Hz, so low pass filtering the signal at a frequency around 1-3 Hz can be applied to remove high frequency noise.

Detecting individual breaths in a sleep recording can be done by using the abdomen RIP signal, the thorax RIP signal, or their sum (RIPsum). Breath onset is defined as the moment when the lungs start filling with air from their functional residual capacity (FRC) causing the chest and abdomen to move and their combined movement corresponding to the increase in volume of the lungs. Functional Residual Capacity is the volume of air present in the lungs at the end of passive expiration and when the chest and abdomen are in a neutral position.

A RIPsum signal of breathing during sleep may be obtained. The RIPsum starts at a lower bound, End/Start, and rises to an upper bound, Midway point, before it falls back down. The rise of the signal indicates the breath onset. A naive or simple method of detecting the breath onset is to look for points where the derivative of the signal changes sign from negative to positive, or when the derivative crosses the zero value from negative to positive and label them as End/Start. Points where the sign of the derivative changes from positive to negative are the Midway points. However, this naive or simple method suffers from misidentification of End/Start points and Midway points in the presence of noise.

In the presence of noise, too many points can be identified as End/Start points or Midway points. To mitigate this one can low-pass filter the signal at a frequency high enough to capture the breathing movement and low enough to remove most noise. A cutoff frequency of, for example, 3 Hz could be used, as it is around ten times higher than the breathing frequency. A second mitigation strategy is to investigate the End/Start points and Midway points and identify points which represent noise. One strategy to combine points is to define a threshold value in the signal amplitude which needs to be passed before defining a new End/Start point or a new Midway point.

A correlation feature is based on the similarity of adjacent breaths. To evaluate their similarity the cross-correlation is used with the coefficient scaling method. The coefficient scaling method normalizes the input signals, so their auto-correlation is 1 at the zero lag. The cross-correlation is calculated for each adjacent pair of breaths and the correlation of the breaths is found as the maximum value of the cross-correlation. The last breath of the previous epoch is included for the correlation calculation of the first breath of the current epoch. The mean and standard deviation are then calculated over each epoch. The RIPSum signal is used for these calculations.

The breath length for each breath is calculated along with the inhalation and exhalation durations. This may be done using the start, end and peak values returned by the breath finder. For each epoch then the mean and standard deviation of these lengths was calculated. The median peak amplitude of the RIPsum signal is also calculated for each breath over an epoch.

The median volume and flow of the inhalation, exhalation and the whole breath are calculated for each breath and then the median of all breaths within each epoch is calculated. Along with that, the median of the amplitude of each breath is calculated and the median value of all breaths within each epoch is calculated. This results in 6 features.

The zero-flow ratio is calculated by locating the exhalation start of each breath. The difference of the amplitude at exhalation and inhalation start is calculated for the abdomen and thorax volume signals and the ratio of the abdomen and thorax values are calculated for each breath. The mean and standard deviation of these values are then calculated for each epoch.

### 3.3—Activity

For the activity features the standard deviation over 30, 20, 10, 5, and 1 second interval is calculated and the maximum and minimum difference over 30, 20, 10, 5, and 1 second interval is as well calculated. The activity features may be calculated using the activity signal. The activity signal is calculated by

$$\frac{d}{dt} \sqrt{{x}^{2} + {y}^{2}}$$

Where x and y are the x and y component, moving in the horizontal plane, of the 3D accelerometer signal.

Some of the features use the Abdomen and Thorax Flow signals which were calculated by numerical differentiation from the volume signals. The features that use the breath-by-breath analysis use it in the same way as the breath features in the chapter 3.2.

The mean and standard deviation of the RIPphase signal are calculated over each 30, 20, 10, 5, and 1 second interval.

Skewness is a measurement on the asymmetry in a statistical distribution. This can be used to look at if the breaths are more skewed to the inhalation part or the exhalation part. It can be seen that the breathing patterns change or how the breathing rhythm changes. The skewness is the 3rd standardized moment and is defined as

$${γ}_{1} = \frac{{μ}_{3}}{{μ}_{2}^{3 / 2}} \qquad 3.3$$

To calculate the skewness of the breath it is interpreted as a histogram. The signal is digitized somehow, for example, by scaling it between 0-100 (a higher number can be used for more precision) and converted to integers. The skewness may be calculated by at least two ways at this point. The first method is to construct a signal that has the given histogram and then use built-in skewness functions. The second method is based on calculating the skewness directly by calculating the third moment and the standard deviation using the histogram as weights. First, a signal is made x=(1, 2, . . . , n−1, n) where n is the length of the original signal. Then the weighted average is calculated with

$$μ = \frac{1}{N} {\sum }_{i = 1}^{n} ( {x}_{i} * {k}_{i} ) \qquad 3.4$$

where k is the original signal N=Σᵢ₌₁ⁿ kᵢ is the weighted length of x. The weighted third moment is then calculated with

$${μ}_{3} = \frac{1}{N} {\sum }_{i = 1}^{N} {{k}_{i} ( {x}_{i} - μ )}^{3} \qquad 3.5$$

and the weighted standard deviation with

$$σ = \sqrt{\frac{1}{N} {\sum }_{i = 1}^{N} {{k}_{i} ( {x}_{i} - μ )}^{2}} \qquad 3.6$$

The skewness is then calculated with equation 3.4

This may be done for each breath and the mean and standard deviation of the breaths within one 30 second epoch are calculated. The skewness is calculated for the abdomen, thorax and RIP volume traces. The RIPSum may be used to obtain locations of each breath.

The ratio of the maximum flow in inhalation and exhalation may be found by first subtracting the mean from the flow signal and then dividing the maximum of the signal with the absolute of the minimum of the signal. The mean of this ratio may be calculated over 30 second epochs. This ratio is both calculated for the abdomen flow and the thorax flow signals.

The time constant of inhalation and exhalation may also be used as features for the classifier. The time constant r is defined as the time it takes the signal to reach half its maximum value. This is done by first subtracting the minimum value from the whole signal so that the minimum value of the signal is at zero. Half the max value is then subtracted so that the half-way point is at 0 and max(f)=−min(f). Taking the absolute value of the signal then results in a V-shaped signal and the halfway point is then found by finding the lowest point of the signal. The formula is as follows:

$$τ = arg {min}_{t} ( abs ( f ( t ) - \frac{{max}_{f} f ( t ) - {min}_{f} f ( t )}{2} ) ) \qquad 3.7$$

The time constant may then be calculated for inhalation and exhalation of each breath and averaged over the epoch. This is calculated on each volume signal and their corresponding flow signal. In total this results in 12 features, but of course more or less features may be used.

Breath length features may also be included, which may be calculated for all volume signals and their corresponding flow signals. First, the peak of the breath is found as the maximum value of the breath. The start of the breath is then found as the minimum value on the left side of the breath and the end as the minimum value on the right side. The inhale, exhale and total length of each breath is then calculated. The breaths are fetched with the breath-by-breath analysis on the RIPSum signal. This results in total of 18 features, but of course more or less features may be used.

### Pre-Processing

The CSV files with the features for each recording may be loaded up in Python. Before any training or classification is started, some pre-processing may be required or preferable. The pre-processing may involve normalizing the features for each recording, to make the features independent of the subject in question. For example, if we have subject A with heart rate of 80±5 bpm and subject B with heart rate 100±10, they cannot be compared directly. To make them comparable we use the z-norm which may be defined as

$$\overline{Z} = \frac{\overline{x} - μ}{σ} \qquad 4.1$$

Where x̅ is a feature vector, y is the mean of the feature vector, and a is the standard deviation of the vector. By using the z-norm, each feature takes the value of 0±1 and they are therefore independent of subjects and are comparable between sleep stages.

The pre-processing also involves converting the labels from strings (‘sleep-wake’, ‘sleep-rem’, ‘sleep-n1’, sleep-n2′, ‘sleep-n3’) to numbers (0, 1, 2, 2, 2). The five given sleep stages may thus be mapped to three stages: 0—wake, 1—REM, 2—NREM. The labels are then one-hot-encoded as required by the neural network architecture. To explain further, if an epoch originally has the label ‘sleep-n2’, it will first be assigned the number 2, and then after one-hot encoding, the label is represented as [0, 0, 1].

### Classifier

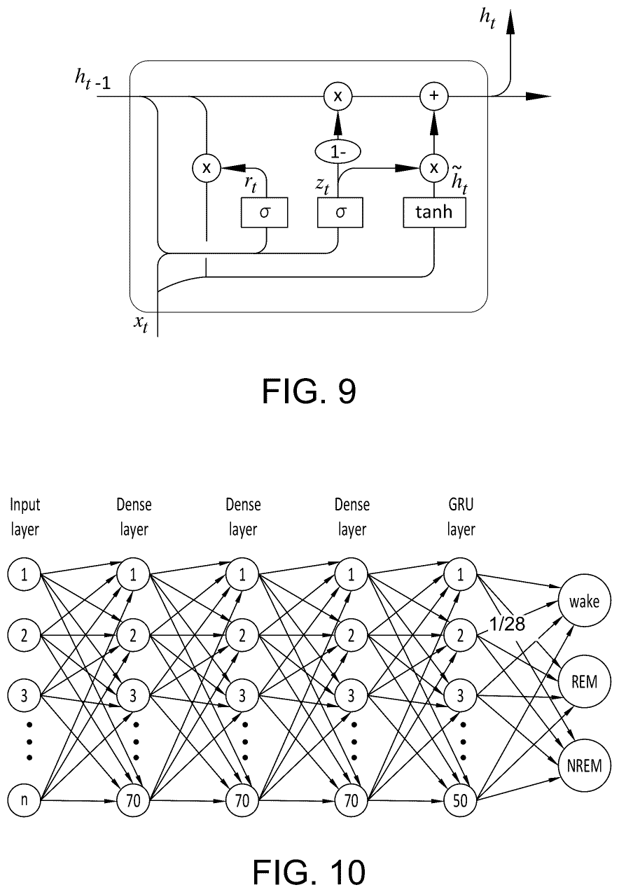

The use of neural networks was considered for the classification task, as neural networks are well suited to learn from large and complex datasets. The use of gated recurrent units (GRU) was considered as gating mechanism to make the classification more time and structure dependent. GRU is a special type of recurrent layer that takes a sequence of data as an input instead of a single instance. GRU provides the network to see the ability to capture the time variance of the data, that is it can see more than just the exact moment it is trying to classify. The structure of a GRU unit can be seen in FIG. 9.

The implementation and training of the neural network was performed in Python, using the Keras machine learning library, with TensorFlow backend. TensorBoard was used to visualize and follow the progress of the training in real-time.

### 5.1—the Architecture of the Final Classifier

After experimenting with different neural network architectures and tuning hyperparameters, a robust classifier was converged on. In this embodiment, the final classifier is a neural network, having three dense layers (each with 70 nodes), followed by a recurrent layer with 50 GRU blocks. The output layer of the network has of 3 nodes, representing for each timestep the class probabilities that the given 30 sec. input window belongs to the sleep stages wake, REM and NREM, respectively. A diagram of an example network can be seen in FIG. 10 where n is the number of features fed to the network.

The classifier may be simplified to a single neural network, with both dense layers and a recurrent layer, whereas the previous classifier was composed of two separate neural networks (a dense one and a recurrent one). Further, early stopping may be introduced to minimize training time and to help reduce overfitting. Learning rate was also changed from being static to dynamic, so it is reduced on plateau. Other hyper-parameters were also changed, such as the dropout rate and the timesteps for the recurrent network. The new model was easier to tune and gave a higher cross-validated F1-score.

Some variations of the structure of the original classifier were tried, including:
1. Skipping the dense pretrained model, keeping the same structure for the RNN model;
2. Use bidirectional LSTM model, instead of a GRU model;
3. Change patience in “Reduce learning rate on plateau”, tried values between 2-10;
4. Tried timesteps=10, future=5;
5. Cascaded binary classifier, where first classify sleep-wake, then classify sleep into nrem/rem.
a. Tried two versions of rem-nrem classification, first using weights=0, performed badly
b. Second training only on rem-nrem, performed better than the first version;
6. Ensembling models trained with different seeds and different features;
7. tanh activation instead of relu in the dense net, for a balanced input to the RNN;
8. Try leaky-relu activation instead of relu, in the dense net.

As used herein, RNN is Recurrent Neural Network a type of an artificial neural network which learns patterns which occur over time. An example of where RNNs are used is in language processing where the order and context of letters or words is of importance.

LSTM is Long-Short Term Memory a type of an artificial neural network which learns patterns which occur over time. The LSTM is a different type of an artificial neural network than RNN which both are designed to learn temporal patterns.

GRU is Gated Recurrent Unit, a building block of RNN artificial neural networks.

Secondly, some variations of the structure of the current classifier described in chapter 5.1 were tried.
1. The dense layer preceding GRU has 50 nodes instead of 70;
2. The GRU layer with 70 nodes instead of 50;
3. Using dropout values from 0.20-0.50 (dropout of 0.22 performed best).

There are many alternative neural network structures that would yield a comparative or similar result. There are even neural network structures, such as Convolutional Neural Networks (CNN), which as noted above can be preferable as such networks can use the raw recorded signals without having to have the features extracted or predetermined as we do here.

The number of layers, number of units, the connection between layers, the types of layers (RNN, LSTM, Dense, CNN, etc), activation functions, and other parameters can all be changed without reducing the performance of the model. Therefore, this disclosure should be not limited to a particular number of layers, number of units, the connection between layers, the types of layers (RNN, LSTM, Dense, CNN, etc.), activation functions, or other parameters that can be changed without reducing the performance of the model

Although the subject matter of this disclosure has been described in language specific to structural features and/or methodological acts, it is to be understood that the subject matter defined in the appended claims is not necessarily limited to the described features or acts described above, or the order of the acts described above. Rather, the described features and acts are disclosed as example forms of implementing the claims.

Additional Validation of (Nox BodySleep2.0) the AI Model of this Disclosure for Predefined Patient Groups

Diagnostics for sleep-disordered breathing (SDB) typically involve labor-intensive, in-laboratory Polysomnography (PSG), accompanied by manual scoring from a sleep technologist. This retrospective study validates the performance of the Nox BodySleep2.0 (herein simply “BodySleep”), a CNN-based AI analysis or algorithm, in conjunction with Home Sleep Apnea Tests (HSAT) for accurate Apnea-Hypopnea index (AHI) classification, across diverse sub-groups.

Data from various sleep centers were used, with a total of N=2477 sleep studies used in the analysis. The subgroups considered for this study were demographics, comorbidities, and medication types. The disclosed “BodySleep” analysis was compared to manual scoring using percentage agreement, predictive values, cohen's kappa, and 95% confidence intervals derived from bootstrapping.

The analysis exhibited strong agreement with manual scoring of AHI classification across most subgroups. For AHI thresholds of 5, 15, and 30, the overall percentage agreement (OPA) was observed to be 95%, 92%, and 95% respectively. Predictive values were also strong, with a positive predictive value (PPV) of 97%, 96%, and 94% for the same AHI thresholds. Cohen's Kappa values ranged from 0.84 to 0.87, indicating substantial agreement, and F1 scores were consistently high, ranging from 0.89 to 0.96.

The “BodySleep” analysis disclosed herein (also referred to as the “Nox BodySleep2.0”) shows to be a viable tool for accurate AHI severity classification in HSAT studies, making it a potential cost-saving and accessible alternative to manual PSG scoring. While it performed consistently well across most subgroups, certain subgroups with limited data may require further investigation.

### Further Introduction

General about Sleep

Approximately one-third of a person's life is spent sleeping, with the average adult requiring 7-9 hours of sleep each night for optimal health and well-being. However, fast-paced society and lifestyle choices can often interfere, resulting in a large portion of adults sleeping fewer hours than recommended. Sleep can be divided into three stages: wakefulness, rapid eye movement sleep (REM), and non-rapid eye movement sleep (NREM), which is subdivided into three stages of N1-N3. During NREM sleep, the brain activity slows down, and physical renewal occurs. During REM sleep, brain activity increases, but the muscles are mostly paralyzed. In healthy adults, these sleep stages appear in a predictable cycle pattern throughout the night. This roughly 90-120 minute cycle can be influenced and disrupted by various factors, that decrease the quality of sleep. Sleep quality is influenced by a range of factors including diet, age, physical activity, medication, genetic factors, environmental factors, sleep duration, and sleep disorders.

### Sleep Disordered Breathing

Sleep-disordered breathing (SDB) refers to a group of sleep disorders characterized by respiratory events that can occur during sleep. Those disorders include, but are not limited to, obstructive sleep apnea (OSA) and central sleep apnea (CSA).

OSA is a common disorder of repeated upper airway collapse during sleep. A complete or partial collapse results in respiratory events of apnea or hypopnea, respectively. An apnea is defined as a 90% reduction in airflow for 10 seconds or more. Hypopnea is defined as ≥30% reduction in airflow for 10 seconds or more, accompanied by either a ≥3% oxygen desaturation or an arousal event. The severity of OSA is determined based on the apnea-hypopnea index (AHI), representing the average number of apneas and hypopnea events per hour of sleep. The severity is determined by the American Academy of Sleep Medicine (AASM) where a score of fewer than 5 apneas per hour is considered a normal score, between 5 and <15 apneas per hour indicates mild OSA, 15 to <30 apneas per hour indicates moderate OSA, and ≥30 apneas per hour indicates severe OSA.

The global prevalence of OSA is estimated to be almost one billion people worldwide. Obesity, male sex, and increased age are the main risk factors of OSA. Common symptoms of OSA include loud snoring, sleep fragmentation, and excessive daytime sleepiness. Patients with OSA are often unaware of their loud snoring and breathing cessations. This lack of symptom awareness, coupled with a lack of education relating to the disorder and healthcare systems mainly focusing on acute illnesses, causes OSA to be severely underdiagnosed and undertreated.

### Sleep Studies

The most comprehensive sleep study is an in-laboratory polysomnography (PSG), also known as a type 1 sleep study, considered the gold standard for assessing sleep and OSA. This study necessitates an overnight visit to a sleep laboratory, where multiple physiological variables are recorded. By accurately determining the sleep stages and respiratory events, these signals allow for precise AHI scoring. Home sleep apnea tests (HSAT) encompass type 2-4 sleep studies allowing the study to be performed at home. A type 2 study uses a portable PSG device, which is typically set up by a sleep technologist in a laboratory, at the patient's home, or in some cases by the patients themselves using detailed guidelines. Self-applied somnography (SAS) and an enriched HSAT test (HSAT+) are notable types of type 2 studies, with SAS utilizing frontal electroencephalogram (EEG) and electrooculography (EOG) signals, and HSAT+ using a reduced EEG, allowing for precise hypopnea scoring. Type 3 and 4 sleep studies include fewer channels. Type 3, also called a polygraph, consists of the same signals as a PSG study, excluding the EEG, EOG, electrocardiogram (ECG), leg electromyography (EMG), and chin EMG. Type 4 sleep studies narrow it further to only one to three channels. However, only one type of type 4 study has been accepted by the AASM as a definitive diagnostic tool. Table 2-1 provides an in-depth comparison of these sleep studies.

TABLE 2-1
Comparison of measurements for different types of sleep studies

| Measurements | Type 1 | Type 2 | Type 3 | Type 4 |
|---|---|---|---|---|
| At home | | X | X | X |
| In a laboratory | X | | | |
| Sleep technologist | X | | | |
| Electro-encephalogram (EEG) | X | X | / | |
| Electro-ocoulogram (EOG) | X | X | / | |
| Electro-cardiogram (ECG) | X | / | / | |
| Chin Electro-myography (EMG) | X | / | / | |
| Leg Electro-myography (EMG) | X | X | / | |
| Respiratory flow | X | X | / | / |
| Nasal vs mouth breathing | X | X | | |
| Respiratory movements (RIP belts) | X | X | X | |
| Oxygen saturation | X | X | X | X |
| Heart rate | X | X | X | X |
| Body position | X | X | X | / |
| Video | X | | | |
| Audio | X | / | / | / |

Generally included: X. Can be included: /. EEG: Electro-encephalogram; EOG: Electro-oculography; ECG: Electro-cardiogram; EMG: Electro-myography; RIP: Respiratory inductance plethysmography.

Using HSAT is a relatively new method of measuring SDB and can be more convenient for patients. It is less expensive than an in-laboratory PSG and allows the patient to sleep in a familiar home setting as opposed to a sleep laboratory. However, limitations of type 3-4 sleep studies include a tendency to underestimate OSA severity, primarily because it calculates the AHI based on total recording time rather than total sleep time. Additionally, the absence of EEG signals in HSAT also impacts their ability to identify arousal, resulting in a potential underestimation of hypopneas. With the integration of machine learning (ML) algorithms or analysis such as BodySleep2.0 disclosed herein, the data from HSAT can be used to score sleep stages and detect arousals, bypassing the need for an EEG. Many patients that could be accurately assessed by an HSAT study, provided it has a mechanism to score sleep stages and detect arousals, are sent for an in-laboratory PSG. Individuals with an AHI <5 from an HSAT study may be invited for an in-lab PSG to confirm the absence of SDB. A positive SDB diagnosis, specifically OSA, is met if either AHI or Respiratory Disturbance Index (RDI) is ≥15, or AHI or RDI ≥5 along with a documented comorbid condition, such as excessive daytime sleepiness. The PSG is unquestionably a vital tool for patients with complicated comorbidities, to make a precise clinical diagnosis. Using new diagnostic techniques could, however, result in better use of the sleep laboratory's current capacities for patients who need those resources. Smart diagnostic devices with built-in automatic data processing algorithms or analysis make it possible to detect SDB more precisely at home without using time-, personnel-, and cost-consuming PSG tests. The gap between the high prevalence of SDB and the limited diagnostic capabilities could be filled by improving the diagnostic accuracy of HSAT by using more sophisticated diagnostic methods than PSG.

Deep Learning with Sleep Studies

Recent studies of automated sleep stages have shown promising results with ML algorithms across large and diverse patient populations. They utilize different classifiers and feature determination methods that have been trained on datasets with thousands of participants. During testing, these algorithms have achieved sleep staging accuracy similar to interrater reliability of manual scoring. Sleep staging is a time-consuming process that requires manual inspection by a sleep technician, in batches of 30-second epochs, of EEG, ECG, and EMG. The manually scored sleep staging is evaluated with inter-rater reliability as quantified by kappa (κ) that reflects epoch-by-epoch agreement above chance. Utilizing PSG data, annotated by sleep technicians, offers an extensive repository of labeled data that proves valuable for training deep learning algorithms to evaluate sleep disorders.

Using AI to determine sleep stages and score respiratory and movement events may reduce the time sleep technologists must spend on PSG scoring, allowing them to devote more time to patient needs. This could potentially lead to savings in both cost and time for patients and clinicians, as well as a reduction of tedious waiting for in-lab PSG. In recent years, deep learning has shown successful implementation in various domains such as image and speech recognition. Convolutional neural networks (CNN) are one of the most widely used forms of deep learning among researchers, due to their ability for autonomous higher-level pattern recognition.

An embodiment termed “Bodysleep2.0”

An embodiment of the method disclosed herein, referred to as the Nox BodySleep™ 2.0 (Nox Medical, Iceland), is based on a deep learning algorithm, which may be based on a convolutional neural network developed by the inventors at Nox Medical, intended to classify 30-second epochs of a type 3 sleep study into the states of REM, NREM, and wake. The analysis was designed to predict changes in autonomic functions correlating with the different sleep stages and arousals.

Examples of the algorithm extract data from actigraphy and respiratory inductance plethysmography (RIP) belts. It uses these signals to differentiate between REM, NREM, and wake, detect arousals, and detect respiratory events and motor activity during sleep that are caused by increased sympathetic activity.

These signals can offer insight into arousal events, which can be used in the identification of hypopneas. Furthermore, these alternative signals can be more easily gathered in an HSAT study, thereby reducing the reliance on manual scoring and decreasing stress on sleep centers. The use of CNNs paired with RIP and actigraphy signals for the automatic scoring of arousal events and detection of hypopneas has proven beneficial, due to the CNN's proficiency in feature extraction. However, the effectiveness of this approach may be compromised due to individual variability, particularly demographics, the presence of a comorbid condition, or medication usage.

### Demographics

Demographics provide insights into population characteristics, enabling researchers to analyze trends and patterns to better understand the healthcare needs of a specific population. By collecting information about sex, age, and body mass index (BMI), it is possible to gain a better understanding of the potential limitations of algorithms that are being applied in healthcare settings. Studies have shown that OSA is more prevalent in males compared to females. Epidemiological studies of OSA excluded females until the early nineties. The prevalence of OSA symptoms seems to differ between males and females. Males generally have a higher snoring index compared to females, who less frequently report snoring. Females instead report symptoms like headaches, fatigue, depression, sleep disturbances, and anxiety, these symptoms are often misdiagnosed as insomnia or depression. The prevalence of OSA among females appears to be somewhat connected to different developmental stages such as puberty, pregnancy, and the postmenopausal state. A study by Koo et al. has focused on female sex hormones, to explain the differences in the prevalence of OSA between the sexes.

In addition to sex, the age of individuals is also known to have an effect on OSA. The importance of early diagnosis, treatment of OSA, and patient management is becoming more apparent. It is especially important for older individuals because they have a poorer perception of their symptoms. Individuals with OSA have exhibited more impairment in physical and cognitive functions compared to individuals without OSA.

Another factor that can affect the prevalence of OSA is physiological properties like weight. Overweight and obesity are significant public health concerns worldwide and the body mass index (BMI) is widely used to measure it. Individuals with obesity are also more likely to have OSA, compared to individuals with lower BMI. Fat distribution in females tends to be more peripheral compared to males and settles around hips, buttocks, and thighs. Excess fat in males tends to accumulate more centrally on the abdomen and neck. The difference in fat distribution between sexes may be a factor in the variation in OSA prevalence, as more adipose tissue settling centrally and around the airway is related to an increased risk of OSA.

Due to the potential differences in symptoms experienced by individuals based on factors such as gender, age, and physiological characteristics, it is crucial to ensure that healthcare solutions are equally effective for all. Therefore, it is essential to validate any new healthcare solution across diverse groups and subgroups to determine areas for improvement or assess its adequacy.

### Comorbidities

The relationship between OSA and various comorbidities is complex, with conditions potentially influencing each other with a bidirectional relationship, increasing both the risk of onset and exacerbation of existing symptoms.

The severity of various comorbid conditions has been found to increase with OSA severity, and studies have reported OSA as an independent risk factor for metabolic, cardiovascular, renal, and mental health disorders, among many others. The interrelation of OSA and its comorbidities is further highlighted by treatment for OSA such as positive airway pressure (PAP) being successful in protecting against the worsening of prognosis, and reducing symptoms of many comorbid disorders. Type 2 diabetes and OSA share risk factors of obesity and aging, and symptoms such as decreased sleep quality Type 2 diabetes may influence respiration and sleep patterns through autonomic dysfunction in upper airway stability. Autonomic neuropathy, which is common in diabetes, may also affect breathing, in addition to other autonomic body functions.

Asthma is an inflammatory condition of the airways that leads to episodes of wheezing, breathlessness, chest tightness, and coughing. These symptoms are worse at night and in the early mornings, with asthma patients often waking up due to these symptoms, leading to frequent sleep fragmentation. Nocturnal asthma may affect respiration and movement signals, due to shortness of breath, variable expiratory airflow limitation, and nasal congestion from chronic rhinosinusitis, a common comorbidity with asthma.

Seasonal allergies may affect respiration through nasal congestion and obstruction, as well as inflammation of the upper airway. Negative pressure in the pharynx from allergic rhinitis may increase nasal resistance, predisposing the upper airway to collapse. Additionally, nasal congestion associated with allergic rhinitis could cause sleep fragmentation, due to increased negative intrathoracic pressure swings interfering with respiration patterns.

Heart failure (HF) has been identified as a risk factor for the development of central sleep apnea (CSA). In patients with HF, Cheyne-Stokes respiration is the most common form of CSA, characterized by periodic cycles of crescendo-decrescendo breathing that result in apnea or hypopnea episodes. Hyperventilation, circulatory delay, and cerebrovascular reactivity have been found to occur during sleep in patients with HF, leading to respiratory instability. This change in respiratory patterns has been suggested to be due to increased respiratory control response to changes in partial pressure of carbon dioxide (PaCO₂). Further complexities to sleep disturbances may arise from comorbid conditions that may subtly influence sleep. For instance, cardiovascular conditions such as atrial fibrillation, heart disease, and hypertension have a high comorbidity rate with SDB. While their influence on respiration or movement during sleep may not be explicit, these conditions are associated with heart rate variability and blood pressure fluctuations that could indirectly affect sleep quality and patterns. Since these conditions may alter sleep dynamics such as respiration or sleep patterns, it is essential that the usage of AI algorithms for sleep scoring is not only accurate for the broader public but also reliable for those with a comorbid condition.

### Medications

Various medications drugs can have profound effects on sleep and its architecture. Beta-blockers are a large group of drugs commonly used to treat hypertension, coronary artery disease, and heart failure. Their effect on the body includes lowering heart rate, blood pressure, and cardiac output. Non-selective beta-blockers also tend to cause contraction of smooth muscles which can lead to bronchoconstriction, a tightening of the airways, in predisposed individuals. This could lead to increased respiratory effort. The effects of beta-blockers on sleep do seem to vary depending on studies and their specific properties.

Antidepressants are another group of drugs that significantly impact sleep by altering physiological patterns of sleep stages, particularly in REM sleep. These include selective serotonin reuptake inhibitors (SSRIs), norepinephrine-dopamine reuptake inhibitors (NDRIs), serotonin antagonist and reuptake inhibitors (SARIs), and serotonin-norepinephrine reuptake inhibitors (SNRIs). Antidepressants can alter sleep quality through various mechanisms, including the activation of serotonergic 5-HT2 receptors and changes in noradrenergic and dopaminergic neurotransmission.

Benzodiazepines (BDZ) are also thought to affect sleep architecture. A very recent systematic review of the literature on BDZ and its effects on sleep reported an increase in time spent in NREM stage 2, a decrease in NREM stage 3 and 4, and also a decrease in REM sleep time in individuals who use BDZ. These changes could potentially lead to concentration difficulties, memory impairment, and weight gain. Studies have also shown, via questionnaire, that withdrawal from BDZ use can improve sleep disturbances and daytime sleepiness over time. Furthermore, BDZ are also thought to affect the arousal threshold. In a study on chronic pain patients using opioids, BDZ seemed to slightly depress respiration but also increased the respiratory arousal threshold resulting in a reduced sleep apnea risk and severity with these patients.

Amphetamine (AMP), atomoxetine (ATX), and methylphenidate (MPH) are stimulant drugs. They are all sympathomimetic drugs that increase noradrenergic and dopaminergic transmission, which impacts blood pressure and heart rate.

Opioids are known to promote respiratory instability. Opioid-induced CSA is today the second most common type of CSA and occurs in up to 24% of opioid users. Research has indicated that severe SDB, specifically CSA, is common in individuals undergoing long-term opioid therapy. These individuals have a higher frequency in central apneas and a lower arousal index than those, not under opioid therapy. The association between opioid use and OSA is somewhat unclear but a recent randomized controlled trial from 2020 reported that morphine altered respiratory control but not other OSA phenotypes such as airway collapsibility, pharyngeal muscle responsiveness, and arousal threshold.

Considering that medications can influence respiratory effort, sleep pattern, sleep onset, and total sleep time, it is important to recognize that medications can impact the results of a sleep study. Therefore it is important to ensure, if algorithms are used for AHI scoring, that they give reliable results, not just for the general population but also for individuals using different types of medications.

### Aim and Objective

The aim of the study was to validate the performance of the analysis, as disclosed herein, (referred to BodySleep2.0 or simply as “BodySleep”), for AHI and AHI categories to manually scored PSGs and enriched home sleep apnea tests (HSATs+) on different subgroups. The subgroups include age, sex, BMI, and different comorbidities and medications. The objective of these investigations is to determine if the algorithm performs well compared to the reference method and if its performance is different in sub-groups that could require further investigations, and data collection, or be considered as a contra-indication.

### Methods

### Data Collection

The data collected included manually scored PSG (Nox A1, Nox Medical, Iceland) and HSAT+(Nox T3, Nox Medical, Iceland) sleep studies along with the individuals' additional information, for example, gender, age, medications, and comorbidities. The studies were conducted by in Georgia, USA, at the Hospitais e Clinicas CUF (Lisbon, Portugal). The necessary local permissions to collect, store, process, and use the data for the purposes of clinical validation were acquired by Nox Medical or their research collaborators.

### Study Population

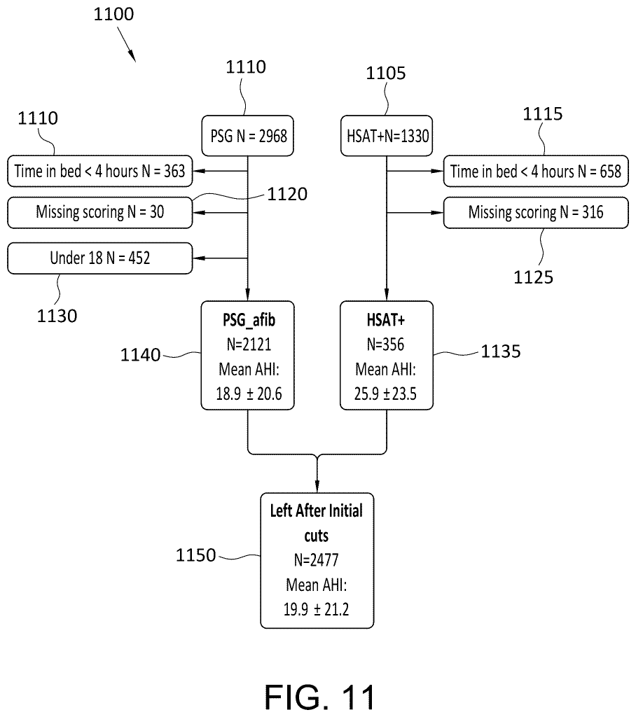

The initial datasets included 4,583 sleep studies: 2,994 PSG and 1,589 HSATs+. After removing duplicates, the datasets were reduced to 2,968 and 1,330 recordings, respectively. Some general exclusion criteria were applied. All daytime recordings were excluded, that is recordings that started before 6:00 pm or after 6:00 am, recordings with shorter than 4 hours of recorded sleep, recordings of individuals under 18 years old, and recordings that contained no manually scored respiratory or arousal events. Individuals were categorized by age, gender, BMI, comorbidities, and medications that could have affected their sleep or respiratory system. All subgroups were looked at individually and recordings with missing values for the subgroups being analyzed were excluded. The flowchart 1100 of FIG. 11 depicts the various exclusion criteria and the final dataset values, following the general exclusion. FIG. 11 shows the general exclusion process for all groups: demographics, comorbidities, and medication, wherein PSG is a Polysomnography; HSAT is a Home Sleep Apnea Test; and AHI is a Apnea-hypopnea Index. At 1101, the PSG data set branch began with N=2968 recordings. At 1110, 363 recordings were excluded due to being less than 4 hours in bed. At 1120, 30 records were excluded for missing scoring. At 1130, 452 recordings were excluded for being under age 18. At 1140, PSG branch remained with 2121 recordings with a mean AHI of 18.9±20.6. The HSAT branch began with 1330 recordings at 1105. At 1115, 658 recordings were excluded for being in bed less than 4 hours. At 1125, 316 recordings were excluded for missing a score. The HSAT branch remained at 1135 with 356 recordings with a mean AHI of 25.9±23.5.

The validation based on demographics was split into three main categories: sex, age, and BMI, which were additionally divided into subgroups. When the categories were analyzed additional recordings had to be excluded in the merged dataset. There were six recordings that had missing sex, nine had missing BMI, and 145 had missing age after the general exclusion criteria had been applied. The recordings that were left to be validated for the sex, BMI, and age category were N=2471, N=2458, and N=2332 respectively. The average AHI after the additional exclusion was 19.9 t 21.1, 20.0±21.2, and 20.0±21.2 respectively for the categories.

For the validation of the comorbidity group, additional N=1490 recordings were excluded after the general exclusion criteria had been applied. A total of N=973 recordings were left to be validated. The average AHI was 15.2±22.3. The individuals that remained reported 14 different comorbidities which were self-reported through a questionnaire. The comorbidities subgroups and the number of individuals in each group can be seen in Table 2-10.

For the validation of the medication group, additional N=2081 individuals were excluded after the general exclusion criteria had been applied. A total of N=396 recordings were left to be validated. The average AHI was 18.5±21.0. The individuals that remained, reported the use of nine different medications through a self-reported questionnaire, five being anti-depressants. The medication subgroups and the number of individuals in each group can be seen in Table 2-13.

### Study Design and Analysis Plan

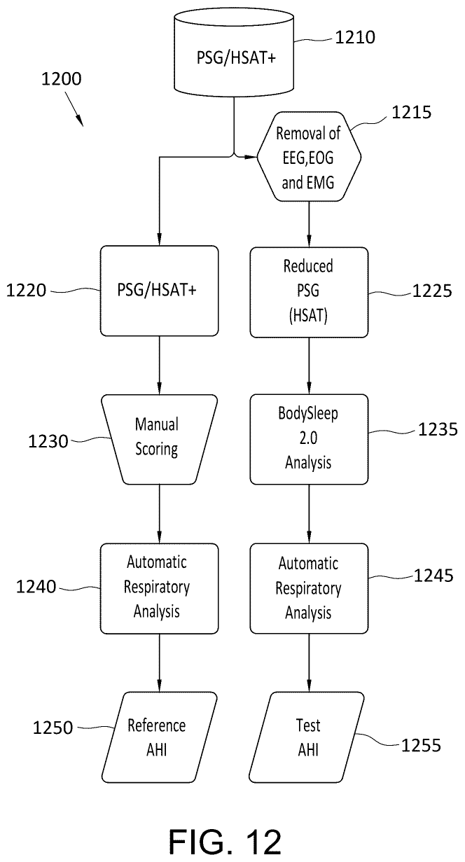

This was a retrospective data analysis study. The data included manually scored PSGs (Nox AI, Nox Medical, Iceland) and HSATs+(Nox T3, Nox Medical, Iceland) sleep studies accompanied by the individuals' demographic data and additional information on their comorbidities and medications. An enriched home sleep apnea test (HSAT+) is a type of home sleep study with additional EEG consisting of one frontal electrode and one ocular electrode, where arousals are scored for more accurate hypopnea scoring. The reference AHI represents the AHI from manually scored PSGs and HSATs+. The test AHI was obtained by removing signals from the PSG and HSAT+ studies and only leaving RIP signals and actigraphy which were then scored by the BodySleep analysis, as disclosed herein. By reducing the PSG and removing signals like EEG, EOG, and ECG, the study resembles an HSAT. This process can be considered further in FIG. 12, which shows a visual representation 1200 of how test and reference AHI were obtained. 1210 shows the dataset with all recordings PSG/HSAT+. At 1215, EEG, EOG, and EMG data are removed for the test branch. At 1225 the dataset of the HSAT branch is reduced (i.e., “Reduced PSG”). At 1235 an embodiment of the disclosed method was performed, termed “BodySleep2.0 Analysis”, following the methods and using the system described herein. At 1245 an automatic respiratory analysis was performed. And at 1255, Test AHI was produced. The reference, regular PSG/HSAT+ branch begins at 1220. The recordings of the full PSG/HSAT+ dataset are manually scored at 1230, at 1240 an automatic respiratory analysis is performed. And at 1250 the reference AHI is produced.

### Statistical Analysis

### Exploratory Parameters

The exploratory parameters were chosen to represent the level of agreement and the diagnostic performance between the manual scoring and the automatic scoring from BodySleep2.0, as disclosed herein.
Positive percentage agreement (PPA), where individuals in the group are identified correctly as having AHI≥5.
Negative percentage agreement (NPA), where individuals in the groups are identified correctly as having AHI <5.
Overall percentage agreement (OPA), where individuals in each group am correctly identified as having either AHI ≥5 or AHI <5.
Positive Predictive Value (PPV), evaluates the proportion of positive AHI ≥5 test results that truly are positive based on OSA prevalence in the general population.
Negative Predictive Value (NPV), evaluates the proportion of negative AHI <5 test results that truly are negative based on OSA prevalence in the general population.

Bootstrapping was used to build a sampling distribution to calculate the 95% confidence intervals for different subgroups. Sampling was done on the sleep study level. Additionally, Bland-Altman plots and Cohen's Kappa were used to assess the level of agreement or reliability between the reference method and an embodiment of the algorithm disclosed herein, the BodySleep2.0 algorithm, and the F1 score was used to evaluate the accuracy and reliability of the classification models' performance.

### Materials

### Technology

This study utilized Python 3.11 as the main programming language, along with libraries such as Pandas 2.0.3 for data manipulation, Matplotlib 3.7.2 for data visualization, Numpy 1.25.2 for data manipulation, and Sklearn 1.3.0 for constructing confusion matrices. Git was employed for version control.

### Results

### Overall Performance

### The Study Cohort

The study included a total of 4298 sleep studies, and after removing duplicates and applying the exclusion criteria for having slept under 4 hours, missing AHI scoring, or being under 18, 2477 were considered eligible for analysis. Among the 2477 eligible participants, there were 1371 men, 1095 women, and 11 unknown, with an average age of 50±17 years. AHI mean score was 20±21 and a median score of 13. Table 2-2 displays descriptive statistics for all the eligible individuals after applying the above-mentioned exclusion criteria.

TABLE 2-2
Descriptive Statistics of Individuals Health Metrics

| | | | | | Interquartile |
|---|---|---|---|---|---|
| Metric | Min | Max | Mean ± Std | Median | range |
| Age [years] | 18 | 99 | 50 ± 17 | 51 | 26 |
| Weight [kg] | 22 | 250 | 93 ± 27 | 89 | 32 |
| Height[cm] | 121 | 206 | 172 ± 10 | 172 | 15 |
| BMI [kg/²] | 0.4 | 84 | 31 ± 9 | 30 | 10 |
| AHI [/h] | 0 | 143 | 20 ± 21 | 13 | 22 |
| Total Sleep | 241 | 627 | 349 ± 57 | 347 | 67 |
| Time [min] | | | | | |
| ODI3 [/h] | 0 | 149 | 17 ± 19 | 10 | 18 |

BMI: Body Mass Index; AHI: Apnea-Hypopnea index; ODI3: 3% Oxygen desaturation index.

### Agreement

Table 2-3 shows the agreement statistics (PPA NPA and OPA) and the 95% confidence intervals (CI) from bootstrapping for PSG and HSAT+ studies at different AHI thresholds. The bounds of the CIs for PPA, NPA, and OPA were very high for all thresholds. As the AHI thresholds increased to 15 and 30, there was a decrease in bounds for PPA accompanied by an increase for NPA.

TABLE 2-3
Agreement on AHI severity classification for AHI ≥
5, AHI ≥ 15 and AHI ≥ 30

| | PPA [%] | NPA [%] | OPA [%] |
|---|---|---|---|
| AHI ≥ 5 | 96 [95, 97] | 91 [89, 94] | 95 [94, 96] |
| AHI ≥ 15 | 87 [85, 89] | 97 [96, 98] | 92 [91, 93] |
| AHI ≥ 30 | 85 [82, 88] | 99 [98, 99] | 95 [95, 96] |

PPV: Positive predictive value; NPV: Negative predictive value; AHI: Apnea-Hypopnea Index; CI: Confidence interval.

### Predictive Value

Table 2-4 displays the initial PPV and NPV values along with their corresponding 95% confidence intervals obtained through bootstrapping. The confidence intervals for both statistics have very high limits for all thresholds. As the AHI thresholds increased, there was a slight decrease in limits for PPV accompanied by an increase for NPV.

TABLE 2-4
Predictive value on AHI severity classification for AHI
≥ 5, AHI ≥ 15 and AHI ≥ 30

| | PPV [%] | NPV [%] |
|---|---|---|
| AHI ≥ 5 | 97 [97, 98] | 87 [84, 89] |
| AHI ≥ 15 | 96 [95, 97] | 89 [88, 91] |
| AHI ≥ 30 | 94 [92, 96] | 96 [95, 97] |

PPV: Positive predictive valus; NPV: Negative predictive value; AHI: Apnea-Hypopnea Index; CI: Confidence interval.

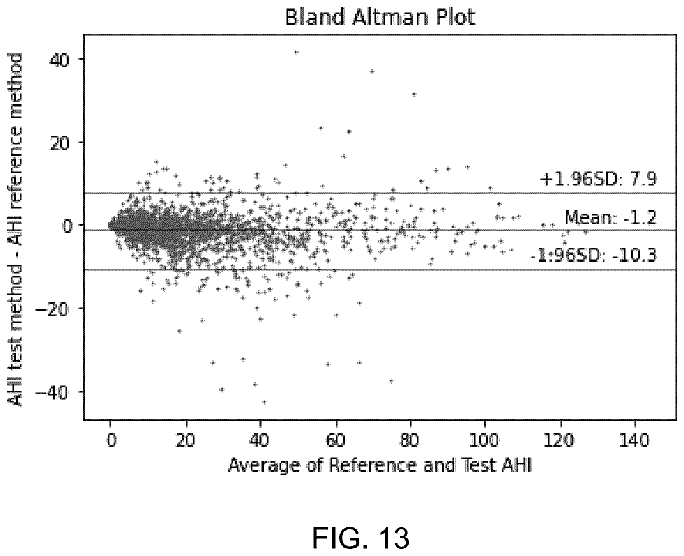

To visualize the agreement between the reference method and the one obtained by the algorithm a Bland-Altman plot was created. The Bland-Altman analysis showed a mean difference of 1.0. The limits of agreement (equal to ±2 standard deviations) were −7.8 and 9.9 AHI. Most data points were centered around where the difference (y-axis) is zero. There was no clear systematic bias where the differences were mainly above or below zero. FIG. 13 shows a Bland Altman plot for the whole cohort.

Table 2-5 summarizes Cohens's Kappa values and F1 scores that were obtained for different AHI severity. Cohen's kappa increased as the AHI increased and ranged from 0.84 to 0.87. The opposite happened to the F1, the score decreased with a higher AHI. It went from 0.96 to 0.89 as the AHI severity decreased.

TABLE 2-5
Comparison of Cohen's Kappa and
F1 Score for different AHI severity

| Condition | Cohen's Kappa | F1 Score |
|---|---|---|
| AHI ≥ 5 | 0.84 | 0.96 |
| AHI ≥ 15 | 0.85 | 0.91 |
| AHI ≥ 30 | 0.87 | 0.89 |

### Confusion Matrices

Table 2-6, show the confusion matrices for AHI thresholds of greater than 5, 15, and 30. They show the true positive (TP), false positive (FP), true negative (TN), and false negative (FN) classifications of different AHI thresholds when the reference AHI scores and the test AHI scores were compared.

TABLE 2-6
Confusion Matrices for AHI ≥ 5, AHI ≥ 15, and AHI ≥ 30

Test method

| | | AHI ≥ 5 | AHI < 5 |
|---|---|---|---|
| Reference | AHI ≥ 5 | 1797 | 82 |
| method | AHI < 5 | 50 | 534 |

Test Method

| | | AHI ≥ 15 | AHI < 15 |
|---|---|---|---|
| Reference | AHI ≥ 15 | 1010 | 149 |
| method | AHI < 15 | 41 | 1263 |

Test Method

| | | AHI ≥ 30 | AHI < 30 |
|---|---|---|---|
| Reference | AHI ≥ 30 | 468 | 85 |
| method | AHI < 30 | 28 | 1882 |

### Demographics

The following table shows the number of individuals in different subgroups for sex, age, and BMI.

TABLE 2-7
Number of the individuals in different subgroups, mean values

| | | | | |
|---|---|---|---|---|
| Size | Age | Height | Weight | BMI |
| [N] | [years] | [cm] | [kg] | [kg/m²] |

Gender

| | | | | | |
|---|---|---|---|---|---|
| Female | 1062 | 47.9 | 165.6 | 83.4 | 30.8 |
| Male | 1264 | 52.0 | 181.1 | 101.6 | 31.7 |

BMI

| | | | | | |
|---|---|---|---|---|---|
| Normal | 504 | 41.8 | 178.1 | 64.0 | 21.8 |
| Overweight | 682 | 53.1 | 172.9 | 82.2 | 27.4 |
| Obese | 1140 | 51.4 | 172.8 | 112.7 | 37.7 |

Age Groups

| | | | | | |
|---|---|---|---|---|---|
| 18-25 | 261 | 20.8 | 170.2 | 76.4 | 26.4 |
| 26-35 | 267 | 30.2 | 171.9 | 96.7 | 32.6 |
| 36-45 | 404 | 40.3 | 174.0 | 97.1 | 32.3 |
| 46-55 | 513 | 50.5 | 176.9 | 100.1 | 33.1 |
| 56-65 | 430 | 59.5 | 174.5 | 95.3 | 31.7 |
| >65 | 451 | 71.9 | 173.7 | 88.0 | 29.7 |

BMI: Body Mass Index

### Bland-Altman

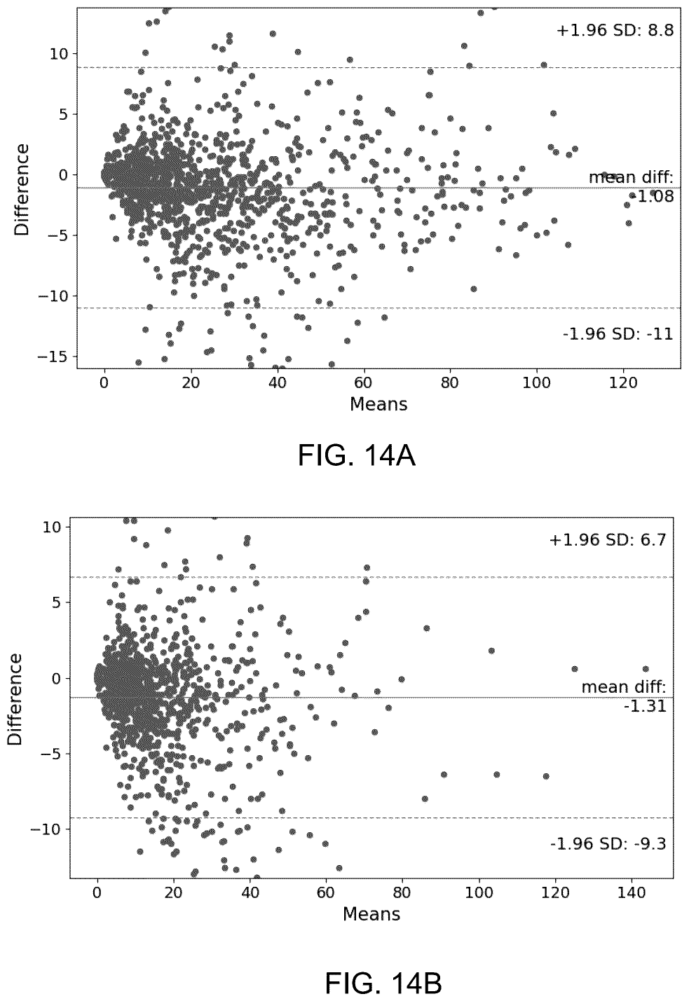

From the Bland Altman plots shown in FIGS. 14, 15, and 16, it can be seen the majority of data points were centered around the zero difference line for all groups and their associated subgroups. Observations outside the limits of the agreement could be potential outliers. The distribution of the differences between the two methods was similar for all subgroups. There was no clear consistent bias where the differences were predominantly above or below zero. The smallest detectable change for all subgroups was similar.

FIGS. 14A and 14B show Bland Altman plots of the reference and test AHI for a) males (FIG. 14A), and b) females (FIG. 14B).

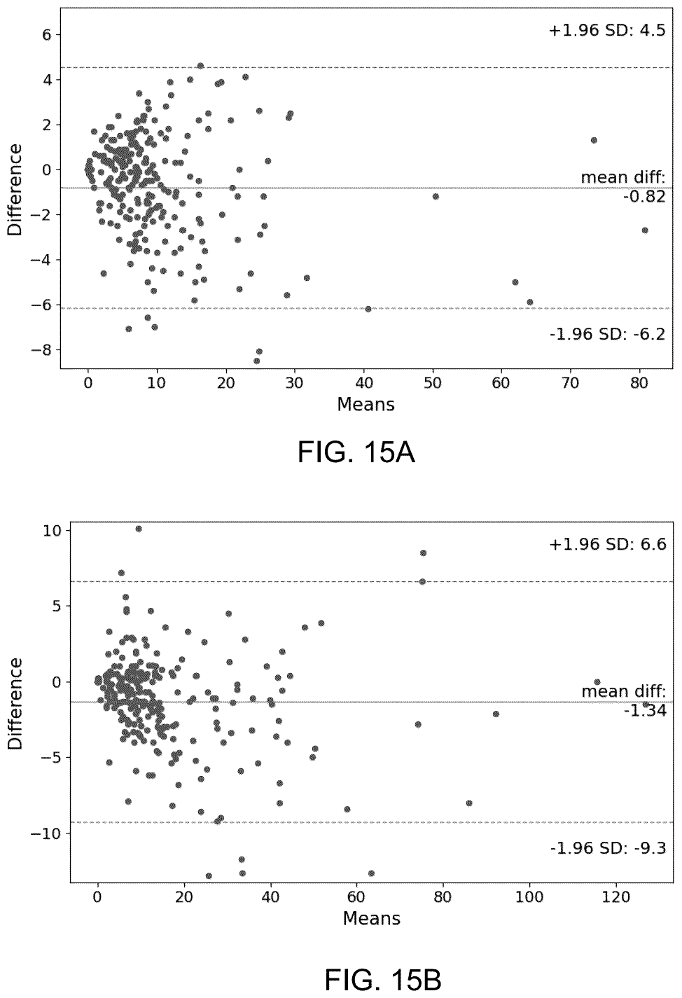

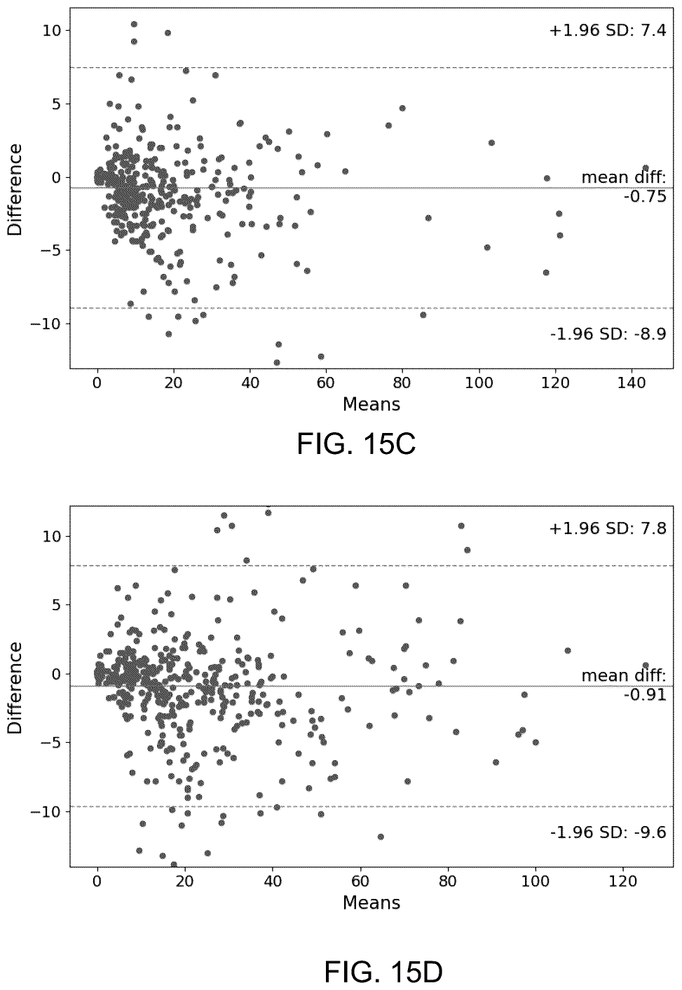

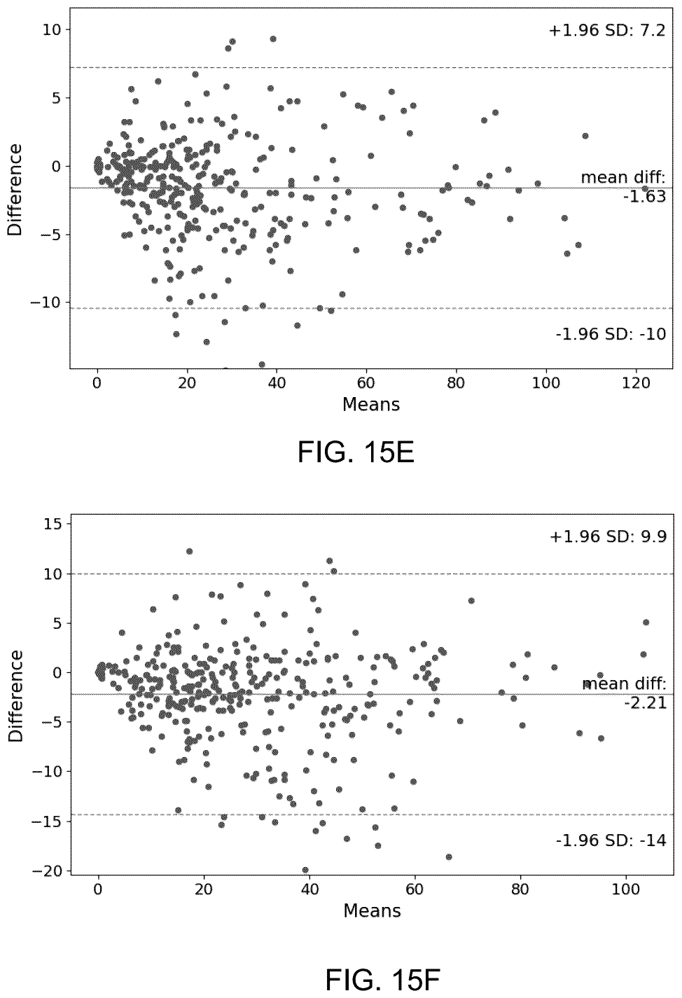

FIGS. 15A-15F show Bland Altman plot of the reference and test AHI for all the different age groups: a) 18-25 years (FIG. 15A), b) 26-35 years (FIG. 15B), c) 36-45 years (FIG. 15C), d) 46-55 years (FIG. 15D), e) 56-65 years (FIG. 15E), and f) 65+ years (FIG. 15F).

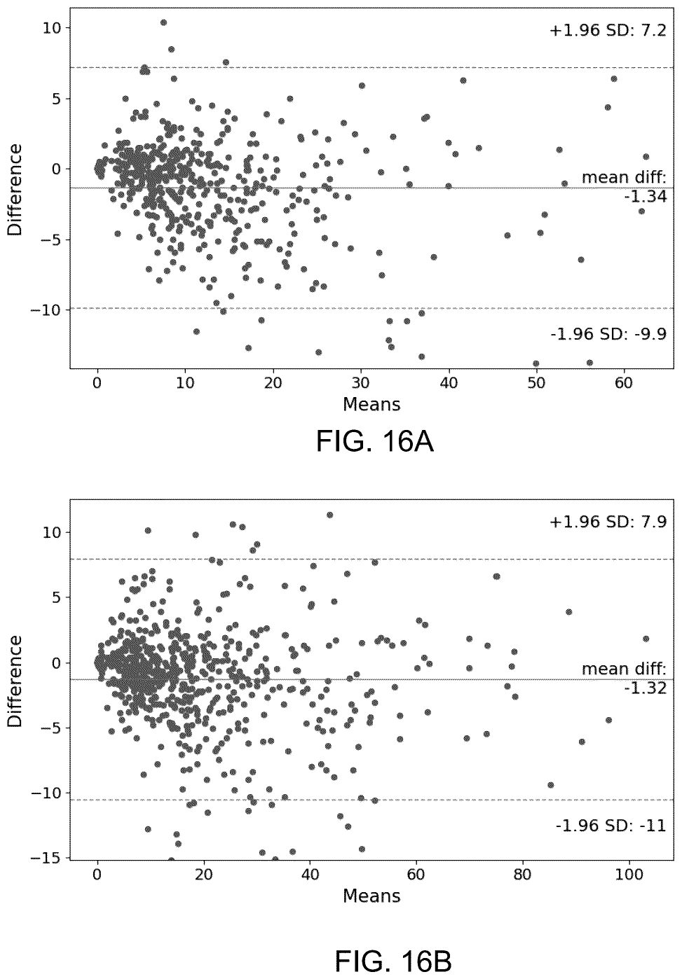

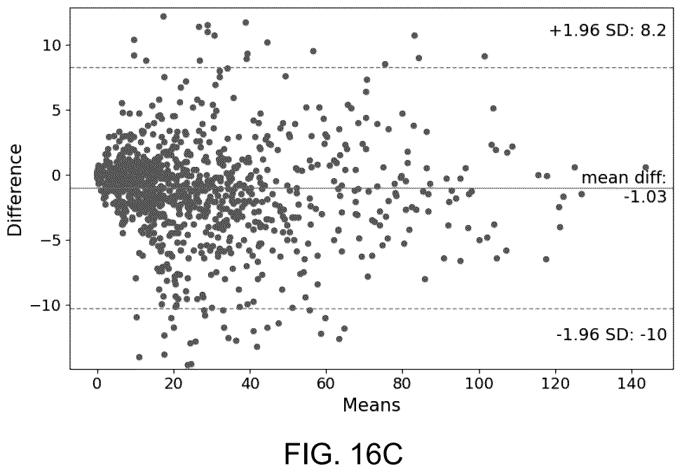

FIGS. 16A-16C show Bland Altman plot of the reference and test AHI for the different BMI groups: a) Normal (BMI <25) (FIG. 16A), b) Overweight (25≤BMI<30) (FIG. 16B), and c) Obese (BMI ≥30) (FIG. 16C).

### Agreement

In this section are the agreement values for the different groups: Sex, age, and BMI. Table 2-8 shows the values of the agreement statistics (PPA, NPA, and OPA) for all subgroups along with their respective 95% CI from bootstrapping.

TABLE 2-8
Agreement on AHI severity classification for AHI ≥ 5 for
males and females in the PSG and HSAT+ dataset

| | | |
|---|---|---|
| PPA [%] | NPA [%] | OPA [%] |

Sex

| | | | |
|---|---|---|---|
| Male | 97.2 [96.2, 98.2] | 92.8 [89.8, 95.5] | 96.2 [95.2, 97.2] |
| Female | 93.5 [91.8, 95.2] | 89.8 [86.2, 93.1] | 92.5 [90.9, 94.1] |

Age groups

| | | | |
|---|---|---|---|
| 18-25 | 92.7 [88.8, 96.2] | 90.1 [82.6, 96.6] | 92.0 [88.5, 95.3] |
| 26-35 | 92.9 [89.2, 96.3] | 82.5 [71.9, 92.0] | 90.7 [87.2, 94.1] |
| 36-45 | 92.7 [89.6, 95.5] | 88.9 [81.6, 95.0] | 91.9 [89.1, 94.4] |
| 46-55 | 96.7 [94.8, 98.3] | 93.5 [88.7, 97.5] | 95.9 [94.2, 97.5] |
| 56-65 | 98.2 [96.6, 99.4] | 97.1 [93.6, 100.0] | 97.9 [96.4, 99.1] |
| >65 | 98.2 [96.6, 99.4] | 98.2 [95.3, 100.0] | 98.2 [96.9, 99.3] |

BMI severity group

| | | | |
|---|---|---|---|
| BMI < 25 | 92.3 [89.6, 94.8] | 84.8 [78.8, 90.5] | 90.4 [87.7, 92.8] |
| 25 ≤ | 95.1 [93.3, 96.8] | 86.8 [81.5, 92.3] | 93.3 [91.4, 95.2] |
| BMI < 30 | | | |
| 30 ≤ BMI | 97.5 [96.4, 98.4] | 97.0 [94.9, 98.7] | 97.4 [96.3, 98.2] |

BMI: Body mass index; PPA: Positive percentage agreement; NPA: Negative percentage agreement; OPA: Overall percentage agreement.

Confidence intervals had high upper and lower bounds for all statistics in all groups and their subgroups. Males had smaller ranges and slightly higher upper and lower bounds on all confidence intervals than females. For all statistics, the upper and lower bounds of the confidence intervals appeared to increase with older age. With rising BMI, the bounds of the confidence intervals for all statistics increased.

### Predictive Value

The predictive values were calculated for each sub-group: Sex, Age, and BMI. Table 2-9 shows the values of the predictive value statistics (PPV and NPV) for all subgroups along with their respective 95% confidence intervals from bootstrapping.

TABLE 2-9
Predictive values for each sex group for AHI ≥
5 in the PSG and the HSAT+ datasets

| | |
|---|---|
| PPV [%] | NPV [%] |

Sex

| | | |
|---|---|---|
| Male | 97.9 [96.9, 98.7] | 90.7 [87.3, 93.6] |
| Female | 96.4 [95.1, 97.6] | 82.5 [78.1, 86.6] |

Age group

| | | |
|---|---|---|
| 18-25 | 96.2 [93.0, 98.8] | 82.1 [73.0, 90.3] |
| 26-35 | 95.1 [92.1, 98.0] | 75.8 [64.6, 86.2] |
| 36-45 | 96.7 [94.4, 98.5] | 77.7 [69.2, 85.4] |
| 46-55 | 97.9 [96.4, 99.2] | 89.8 [84.6, 94.7] |
| 56-65 | 99.1 [97.9, 100.0] | 94.4 [89.5, 98.2] |
| >65 | 99.4 [98.4, 100.0] | 94.9 [90.4, 98.3] |

BMI severity group

| | | |
|---|---|---|
| BMI < 25 | 94.7 [92.4, 96.7] | 79.1 [72.6, 85.4] |
| 25 ≤ BMI < 30 | 96.4 [94.8, 97.9] | 82.5 [76.3, 88.1] |
| 30 ≤ BMI | 99.0 [98.3, 99.6] | 92.6 [89.5, 95.3] |

BMI: Body mass index; PPV: Positive predictive value; NPV: Negative predictive value.

Confidence intervals had high upper and lower bounds for both statistics (PPV and NPA) in all subgroups. The bounds of the confidence intervals for NPV were slightly lower than for PPV for all subgroups. The confidence intervals have lower limitations for NPV when comparing males to females in terms of sex. With regard to age, the predictive value seemed to increase with older age. With regard to BMI, the predictive value appeared to rise with higher BMI.

### Comorbidities

Table 2-10 depicts descriptive statistics for different comorbidities. Comorbidities groups with fewer than 10 individuals were not analyzed due to a lack of data. Additionally, the individuals with a BMI ≥30 were considered obese.

TABLE 2-10
Means of comorbidity subgroups

| | Size | Age | Height | Weight | BMI |
|---|---|---|---|---|---|
| Subgroup | [N] | [years] | [cm] | [kg] | [kg/m²] |
| ADHD | 22 | 32.8 | 174.6 | 94.1 | 30.2 |
| Anxiety | 10 | 60.3 | 172.1 | 95.0 | 32.3 |
| Asthma | 46 | 41.1 | 170.8 | 97.4 | 33.4 |
| Atrial fibrillation | 59 | 64.0 | 172.6 | 90.3 | 30.2 |
| Diabetes | 59 | 46.6 | 171.9 | 107.7 | 36.3 |
| Heart disease | 46 | 64.0 | 174.6 | 101.8 | 33.2 |
| Heart failure | 13 | 62.3 | 173.9 | 112.9 | 37.1 |
| Hypertension | 217 | 56.4 | 174.3 | 108.6 | 35.7 |
| Low testosterone | 44 | 50.3 | 188.8 | 103.7 | 32.4 |
| Obesity | 1213 | 51.4 | 173.0 | 112.5 | 37.6 |
| Gastrointestinal reflux | 255 | 53.5 | 171.6 | 93.8 | 31.7 |
| Seasonal allergies | 58 | 30.3 | 177.3 | 116.5 | 37.1 |
| Seizures | 19 | 40.2 | 171.3 | 86.6 | 29.2 |

ADHD: Attention deficit hyperactivity disorder; BMI: Body mass index.

### Bland-Altman

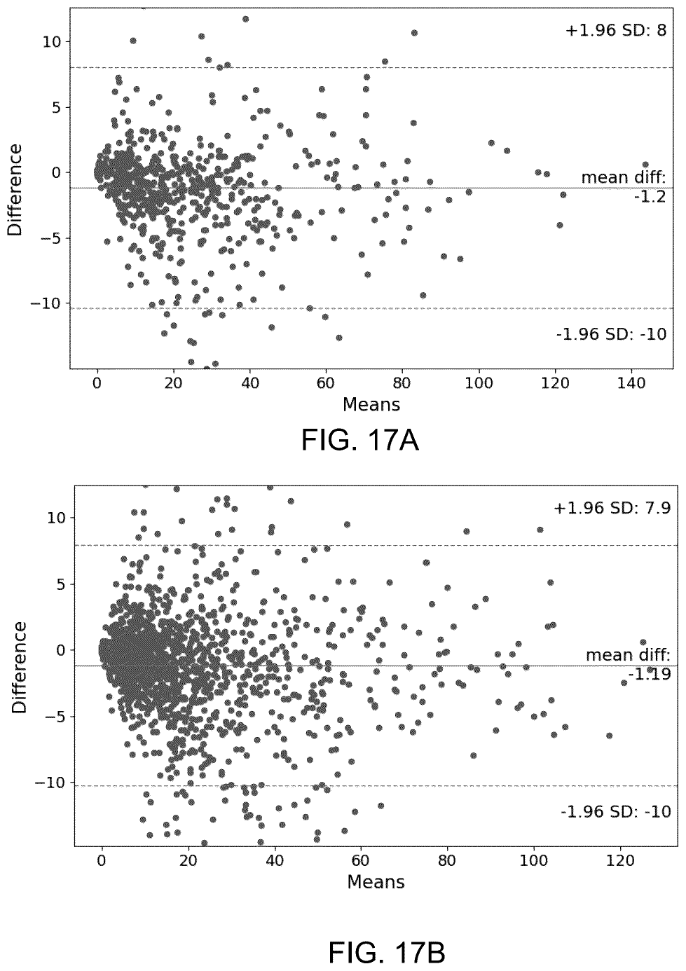

The Bland-Altman plot in FIGS. 17A and 17B show that the majority of data points were centered around the zero difference line for both groups with comorbidity and the group without a known comorbidity. The mean difference for both groups was similar and the same applies to the standard deviation. Data points outside the limits of the agreement could be potential outliers. The distribution of the differences between the two methods was similar for all subgroups. There was no clear consistent bias where the differences were predominantly above or below zero. FIGS. 17A and 17B show a Bland-Altman plot showing agreement for individuals with some comorbidity (FIG. 17A) and individuals without known comorbidities (FIG. 17B).

### Agreement

Table 2-11 shows the PPA, NPA, and OPA for each comorbidity as well as the 95% CI from bootstrapping. The limits of the CIs for PPA were overall high. For heart failure, the lower bound of the CI was the lowest, or 66.7%, and for the low testosterone group, or 84.0%. Other comorbidity groups had a lower bound around 90.0% that ranged in many cases to 100.0%. Regarding the NPA, the lower bounds of the CIs were lowest for heart failure, seasonal allergies, ADHD, and anxiety. The OPA was high for all the groups and the lower bound of the CI was over 85%, except for anxiety and heart failure. The upper bounds of the CIs were high in all cases.

TABLE 2-11
Agreement on AHI severity classification for AHI ≥ 5 for different comorbidities

| Comorbidities | PPA [%] | NPA [%] | OPA [%] |
|---|---|---|---|
| ADHD | 100.0 [100.0, 100.0] | 100.0 [0.0, 100.0] | 100.0 [100.0, 100.0] |
| Anxiety | 100.0 [100.0, 100.0] | 75.0 [0.0, 100.0] | 90.0 [66.7, 100.0] |
| Asthma | 100.0 [100.0, 100.0] | 100.0 [100.0, 100.0] | 100.0 [100.0, 100.0] |
| Atrial fibrillation | 96.4 [90.9, 100.0] | 100.0 [100.0, 100.0] | 96.6 [91.4, 100.0] |
| Diabetes | 96.0 [89.7, 100.0] | 100.0 [100.0, 100.0] | 96.6 [91.3, 100.0] |
| Heart disease | 97.1 [90.6, 100.0] | 83.3 [57.1, 100.0] | 93.5 [85.6, 100.0] |
| Heart failure | 90.0 [66.7, 100.0] | 66.7 [0.0, 100.0] | 84.6 [62.5, 100.0] |
| Hypertension | 98.2 [95.9, 100.0] | 92.0 [83.9, 98.3] | 96.8 [94.3, 99.0] |
| Low testosterone | 93.8 [84.0, 100.0] | 100.0 [100.0, 100.0] | 95.5 [88.5, 100.0] |
| Obesity | 97.5 [96.4, 98.4] | 96.6 [94.4, 98.6] | 97.3 [96.3, 98.1] |
| Gastrointestinal | 94.7 [91.3, 97.8] | 93.9 [87.4, 100.0] | 94.5 [91.6, 97.1] |
| Reflux | | | |
| Seasonal Allergies | 98.1 [93.7, 100.0] | 25.0 [0.0, 100.0] | 93.1 [86.2, 98.5] |
| Seizures | 100.0 [100.0, 100.0] | 100.0 [100.0, 100.0] | 100.0 [100.0, 100.0] |

AHI: Apnea-Hypopnea index; ADHD: Attention deficit hyperactivity disorder; PPA: Positive percentage agreement; NPA: Negative percentage agreement; OPA: Overall percentage agreement

### Predictive Value

Table 2-12 shows the PPV and NPV for each comorbidity and their 95% CI from bootstrapping. Anxiety had the lowest PPV value of 85.7% with a 95% CI of [50-100]% and a NPV of 100.0% with a 95% CI of [0.0-100.0]%. This could be explained by the lack of individuals with anxiety. The highest PPV was 100.0% with a 95% CI of [100-100] % for ADHD, asthma, atrial fibrillation, diabetes, low testosterone, and seizures. These high values could also be explained by the lack of individuals with AHI 55 and the comorbidities mentioned above. As for the other reported comorbidities, the limits of the confidence intervals for PPV and NPV were high for all comorbidities except for Heart failure with an NPV of 66.7% with a CI of [0-100]%. This could also be explained by the lack of individuals with Heart failure.

TABLE 2-12
Predictive value - Positive Predictive Value
(PPV) and Negative Predictive Value (NPV)

| Medication | PPV [%] | NPV [%] |
|---|---|---|
| ADHD | 100.0 [100.0, 100.0] | 100.0 [0.0, 100.0] |
| Anxiety | 85.7 [50.0, 100.0] | 100.0 [0.0, 100.0] |
| Asthma | 100.0 [100.0, 100.0] | 100.0 [100.0, 100.0] |
| Atrial fibrillation | 100.0 [100.0, 100.0] | 66.7 [25.0, 100.0] |
| Diabetes | 100.0 [100.0, 100.0] | 81.8 [55.6, 100.0] |
| Heart disease | 94.3 [85.3, 100.0] | 90.9 [71.4, 100.0] |
| Heart failure | 90.0 [66.7, 100.0] | 66.7 [0.0, 100.0] |
| Hypertension | 97.6 [95.0, 99.5] | 93.9 [86.4, 100.0] |
| Low testosterone | 100.0 [100.0, 100.0] | 85.7 [66.7, 100.0] |
| Obesity | 98.9 [98.1, 99.6] | 92.6 [89.3, 95.3] |
| Gastrointestinal Reflux | 97.8 [95.4, 100.0] | 86.1 [77.3, 93.5] |
| Seasonal Allergies | 94.6 [88.1, 100.0] | 50.0 [0.0, 100.0] |
| Seizures | 100.0 [100.0, 100.0] | 100.0 [100.0, 100.0] |

ADHD: Attention deficit hyperactivity disorder; PPV: Positive predictive value; NPV: Negative predictive value.

### Medications

### The Study Cohort

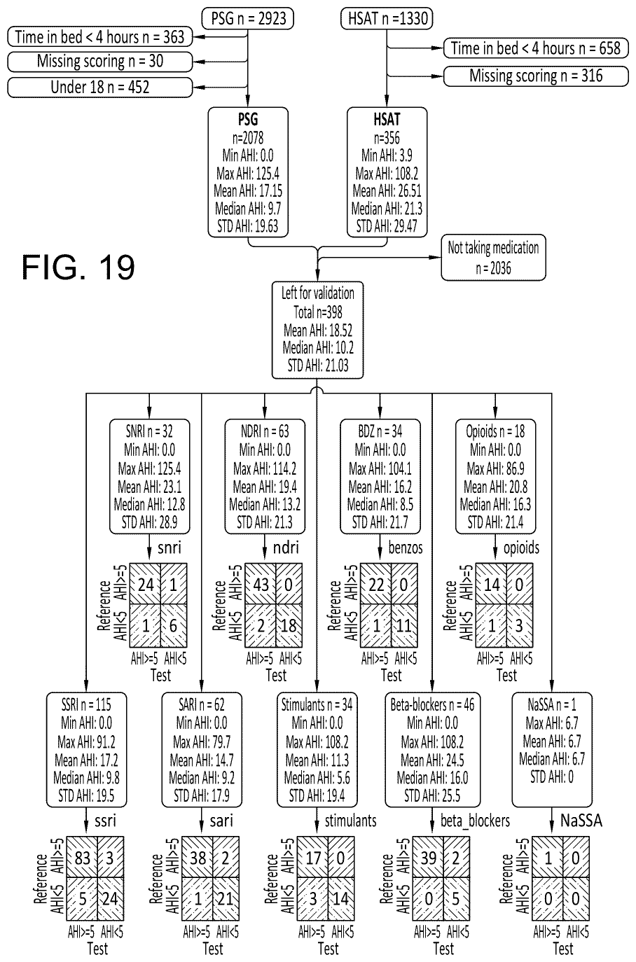

The characteristics of each subgroup can be seen in Tables 2-13, 2-21, and 2-22. Only one individual reported the use of NaSSA. That subgroup was not evaluated further due to its small size. FIG. 19 is provided to show a flowchart used for defining the medication study population.

TABLE 2-13
Characteristics of the individuals
in different subgroups, mean values

| | Size | Age | Height | Weight | BMI |
|---|---|---|---|---|---|
| Subgroup | [N] | [years] | [cm] | [kg] | [kg/m²] |
| BDZ | 34 | 58.2 | 172.8 | 97.1 | 32.3 |
| Beta-blockers | 46 | 63.5 | 174.6 | 101.8 | 33.3 |
| NDRI | 63 | 45.2 | 176.1 | 95.2 | 31.9 |
| Opioids | 18 | 59.6 | 183.8 | 85.0 | 28.1 |
| SARI | 62 | 46.9 | 173.2 | 94.3 | 31.5 |
| SNRI | 32 | 63.7 | 167.6 | 85.9 | 30.1 |
| SSRI | 115 | 38.1 | 170.3 | 92.6 | 31.7 |
| Stimulants | 34 | 35.4 | 170.8 | 83.4 | 28.8 |

BDZ: Benzodiazepine; NDRI: Norepinephrine-dopamine reuptake inhibitor; SARI: Serotonin antagonist and reuptake inhibitor; SNRI: Serotonin and norepinephrine reuptake inhibitor; SSRI: Selective serotonin reuptake inhibitors BMI: Body mass index.

### Bland-Altman

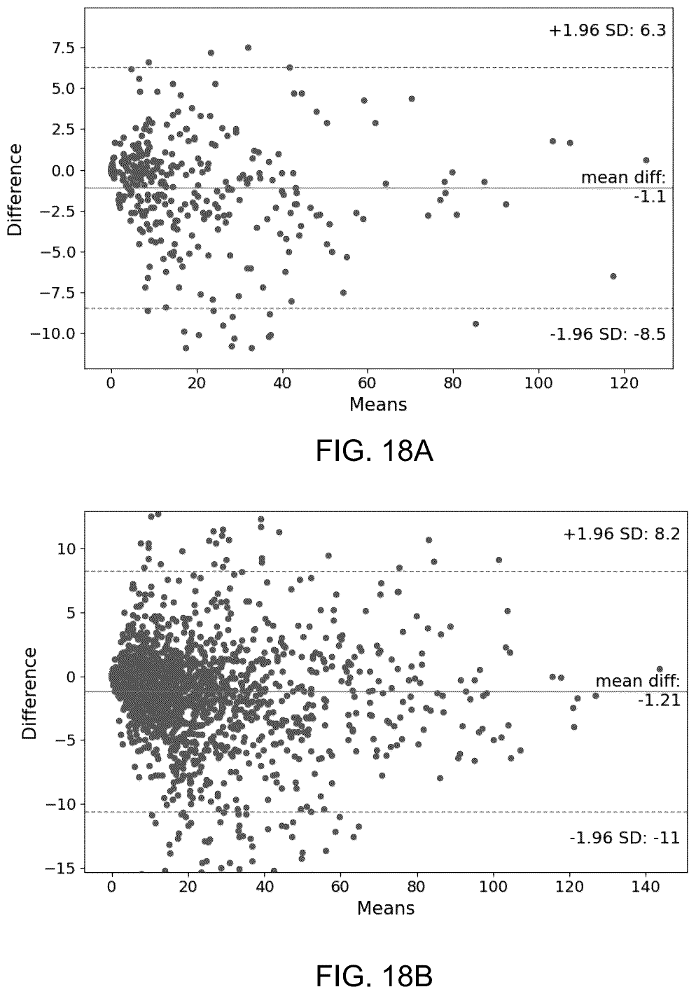

To visualize the difference in agreement between the BodySleep analysis method as disclosed herein and the reference method, two Bland-Altman plots were made, one for individuals taking any medication and another one for individuals who did not. The plots, which can be seen in FIGS. 18A and 18B, which show that most of the observations were inside the limit of the agreement, with fewer observations outside the agreement for medication takers. FIGS. 18A and 18B show a Bland-Altman plot showing agreement for individuals taking medication (FIG. 18A) and individuals not taking medication (FIG. 18B).

### Agreement

Table 2-14 shows the PPA, NPA, and OPA for each subgroup with 95% confidence interval from bootstrapping. The results show that BodySleep2.0 disclosed herein had a high agreement with the reference scoring from the data. The OPA calculated for the different subgroups is high for all medications. Overall agreement has an upper bound over 90% with the standard error of <6 and a lower bound of over 80% for all medications. Additionally, participants taking medication had a substantial agreement between the two scoring methods with a strong Cohen's kappa of 0.86 and an F1 score of 0.96

TABLE 2-14
Agreement on AHI severity classification
for AHI ≥ 5 for different medications

| Medi- | PPA | NPA | OPA |
|---|---|---|---|
| cation | [%] | [%] | [%] |
| BDZ | 95.7 [85.7, 100.0] | 100.0 [100.0, 100.0] | 97.1 [90.3, 100.0] |
| Beta- | 100.0 [100.0, 100.0] | 71.4 [33.3, 100.0] | 95.7 [88.6, 100.0] |
| blockers | | | |
| NDRI | 95.6 [88.9, 100.0] | 100.0 [100.0, 100.0] | 96.8 [91.8, 100.0] |
| Opioids | 93.3 [76.9, 100.0] | 100.0 [0.0, 100.0] | 94.4 [81.2, 100.0] |
| SARI | 97.4 [91.8, 100.0] | 91.3 [78.3, 100.0] | 95.2 [89.5, 100.0] |
| SNRI | 96.0 [87.5, 100.0] | 85.7 [50.0, 100.0] | 93.8[84.3, 100.0] |
| SSRI | 94.3 [88.9, 98.8] | 88.9 [75.3, 100.0] | 93.0 [88.0, 97.3] |
| Stimulants | 85.0 [68.2, 100.0] | 100.0 [100.0, 100.0] | 91.2 [80.2, 100.0] |

BDZ: Benzodiazepine; NDRI: Norepinephrine-dopamine reuptake inhibitor; SARI: Serotonin antagonist and reuptake inhibitor; SNRI: Serotonin and norepinephrine reuptake inhibitor; SSRI: Selective serotonin reuptake inhibitors; PPA: Positive percentage agreement; NPA: Negative percentage agreement; OPA: Overall percentage agreement.

### Predictive Value

Table 2-15 shows the PPV and NPV values along with their respective 95% CI for each subgroup as well as the original value of each statistic before bootstrapping. The confidence intervals for PPV had high bounds for all subgroups.

All subgroups of medications, except SARIs and beta-blockers, had lower bounds for NPV compared to PPV.

TABLE 2-15
Predictive value - Positive Predictive Value
(PPV) and Negative Predictive Value (NPV)

| Medication | PPV [%] | NPV [%] |
|---|---|---|
| BDZ | 100.0 [100.0, 100.0] | 91.7 [71.4, 100.0] |
| Beta-blockers | 95.1 [87.5, 100.0] | 100.0 [100.0, 100.0] |
| NDRI | 100.0 [100.0, 100.0] | 90.0 [75.0, 100.0] |
| Opioids | 100.0 [100.0, 100.0] | 75.0 [0.0, 100.0] |
| SARI | 95.0 [87.5, 100.0] | 95.5 [85.7, 100.0] |
| SNRI | 96.0 [87.3, 100.0] | 85.7 [50.0, 100.0] |
| SSRI | 96.5 [92.4, 100.0] | 82.8 [67.9, 96.0] |
| Stimulants | 100.0 [100.0, 100.0] | 82.4 [61.5, 100.0] |

BDZ: Benzodiazepine; NDRI: Norepinephrine-dopamine reuptake inhibitor; SARI: Serotonin antagonist and reuptake inhibitor; SNRI: Serotonin and norepinephrine reuptake inhibitor; SSRI: Selective serotonin reuptake inhibitors; PPV: Positive predictive value; NPV: Negative predictive value.

TABLE 2-16
Algorithm Performance with patients taking medication

| Condition | Cohen's Kappa | F1 Score |
|---|---|---|
| AHI ≥ 5 | 87.0 | 96.3 |
| AHI ≥ 15 | 86.4 | 91.8 |
| AHI ≥ 30 | 88.2 | 90.6 |

TABLE 2-17
Algorithm Performance for non-medicated patients

| Condition | Cohen's Kappa | F1 Score |
|---|---|---|
| AHI ≥ 5 | 85.0 | 96.4 |
| AHI ≥ 15 | 83.8 | 91.0 |
| AHI ≥ 30 | 85.7 | 88.7 |

Lastly, relating to comorbidities and medications, Tables 2-18, 2-19, 2-20, and 2-21 are provided to show characteristics of the individuals in different subgroups and mean values relating to the data of the sleep studies as used herein.

TABLE 2-18
Characteristics of the individuals
in different subgroups, mean values

| | AHI | Arousals | Respiratory | TST |
|---|---|---|---|---|
| Subgroup | [/h] | [/h] | events [/h] | [min] |
| ADHD | 21.3 | 7.5 | 13.9 | 355.2 |
| Anxiety | 18.9 | 4.0 | 14.8 | 443.4 |
| Asthma | 16.3 | 4.7 | 11.6 | 352.8 |
| Atrial fib | 28.3 | 11.2 | 17.0 | 368.4 |
| Diabetes | 25.9 | 6.1 | 19.8 | 334.7 |
| Heart disease | 23.7 | 8.4 | 15.3 | 332.2 |
| Heart failure | 17.8 | 7.0 | 10.8 | 333.6 |
| Hypertension | 24.4 | 8.5 | 15.9 | 338.4 |
| Low testosterone | 22.3 | 8.6 | 13.8 | 342.1 |
| Obesity | 23.2 | 7.7 | 15.6 | 342.8 |
| Reflux | 17.6 | 4.2 | 13.5 | 337.8 |
| Seasonal | 31.2 | 10.6 | 20.6 | 371.7 |
| Seizures | 17.0 | 6.0 | 10.9 | 351.9 |

TABLE 2-19
Characteristics of the individuals
in different subgroups, mean values

| | Min | Max | Mean | Median | STD |
|---|---|---|---|---|---|
| Subgroup | AHI | AHI | AHI | AHI | AHI |
| ADHD | 0.0 | 115.6 | 21.3 | 10.7 | 26.8 |
| Anxiety | 1.8 | 46.9 | 18.9 | 12.8 | 17.7 |
| Asthma | 0.0 | 79.4 | 16.3 | 12.6 | 15.6 |
| Atrial fib | 0.0 | 109.8 | 28.3 | 25.3 | 22.4 |
| Diabetes | 0.0 | 108.2 | 25.9 | 15.7 | 26.1 |
| Heart disease | 0.0 | 84.7 | 23.7 | 18.0 | 23.9 |
| Heart failure | 0.0 | 62.6 | 17.8 | 10.0 | 19.4 |
| Hypertension | 0.0 | 144.0 | 24.4 | 16.6 | 26.3 |
| Low testosterone | 0.0 | 119.1 | 22.3 | 15.8 | 25.7 |
| Obesity | 0.0 | 144.0 | 23.2 | 15.7 | 24.7 |
| Reflux | 0.0 | 121.2 | 17.6 | 11.2 | 19.6 |
| Seasonal | 0.0 | 117.7 | 31.2 | 20.7 | 29.4 |
| Seizures | 0.0 | 85.4 | 17.0 | 9.3 | 21.8 |

TABLE 2-20
Characteristics of the individuals
in different subgroups, mean values

| | AHI | Arousals | Respiratory | TST |
|---|---|---|---|---|
| Subgroup | [/h] | [/h] | events [/h] | [min] |
| BDZ | 16.2 | 4.6 | 11.6 | 338.3 |
| Beta-blockers | 24.5 | 6.4 | 18.1 | 338.3 |
| NDRI | 19.4 | 3.3 | 16.2 | 341.2 |
| Opioids | 20.8 | 2.8 | 18.0 | 344.3 |
| SARI | 14.7 | 3.7 | 11.0 | 330.0 |
| SNRI | 23.1 | 7.5 | 15.6 | 351.6 |
| SSRI | 17.0 | 4.3 | 12.7 | 350.0 |
| Stimulants | 11.3 | 3.7 | 7.7 | 364.0 |

TABLE 2-21
Characteristics of the individuals
in different subgroups, mean values

| | Min | Max | Mean | Median | STD |
|---|---|---|---|---|---|
| Subgroup | AHI | AHI | AHI | AHI | AHI |
| BDZ | 0.0 | 104.1 | 16.2 | 8.5 | 21.7 |
| Beta-blockers | 0.0 | 108.2 | 24.5 | 16.0 | 25.5 |
| NDRI | 0.0 | 114.2 | 19.4 | 13.2 | 21.3 |
| Opioids | 0.0 | 86.9 | 20.8 | 16.3 | 21.4 |
| SARI | 0.0 | 79.7 | 14.7 | 9.2 | 17.9 |
| SNRI | 0.0 | 125.4 | 23.1 | 12.8 | 28.9 |
| SSRI | 0.0 | 91.2 | 17.0 | 9.8 | 19.5 |
| Stimulants | 0.0 | 108.2 | 11.3 | 5.6 | 19.4 |

Confusions Matrices: To aid in the understanding of the data set considered herein, the following confusions matrices are provided.

TABLE 2-22
Confusions Matrix for males

BodySleep 2.0

| | | AHI ≥ 5 | AHI < 5 |
|---|---|---|---|
| Manually scored | AHI ≥ 5 | 1024 | 29 |
| | AHI < 5 | 22 | 283 |

BodySleep 2.0

| | | AHI ≥ 15 | AHI < 15 |
|---|---|---|---|
| Manually scored | AHI ≥ 15 | 651 | 77 |
| | AHI < 15 | 28 | 602 |

BodySleep 2.0

| | | AHI ≥ 30 | AHI < 30 |
|---|---|---|---|
| Manually scored | AHI ≥ 30 | 335 | 55 |
| | AHI < 30 | 22 | 946 |

TABLE 2-23
Confusion Matrix for females

BodySleep 2.0

| | | AHI ≥ 5 | AHI < 5 |
|---|---|---|---|
| Manually scored | AHI ≥ 5 | 776 | 54 |
| | AHI < 5 | 29 | 254 |

BodySleep 2.0

| | | AHI ≥ 15 | AHI < 15 |
|---|---|---|---|
| Manually scored | AHI ≥ 15 | 356 | 73 |
| | AHI < 15 | 13 | 671 |

BodySleep 2.0

| | | AHI ≥ 30 | AHI < 30 |
|---|---|---|---|
| Manually scored | AHI ≥ 30 | 130 | 30 |
| | AHI < 30 | 6 | 947 |

TABLE 2-24
Confusion Matrix for BMI severity: Normal (25 < BMI)

BodySleep 2.0

| | | AHI ≥ 5 | AHI < 5 |
|---|---|---|---|
| Manually scored | AHI ≥ 5 | 372 | 31 |
| | AHI < 5 | 21 | 117 |

BodySleep 2.0

| | | AHI ≥ 15 | AHI < 15 |
|---|---|---|---|
| Manually scored | AHI ≥ 15 | 122 | 38 |
| | AHI < 15 | 10 | 371 |

BodySleep 2.0

| | | AHI ≥ 30 | AHI < 30 |
|---|---|---|---|
| Manually scored | AHI ≥ 30 | 28 | 14 |
| | AHI < 30 | 3 | 496 |

TABLE 2-25
Confusion Matrix for BMI severity: Overweight (25 ≤ MBI < 30)

BodySleep 2.0

| | | AHI ≥ 5 | AHI < 5 |
|---|---|---|---|
| Manually scored | AHI ≥ 5 | 539 | 28 |
| | AHI < 5 | 20 | 132 |

BodySleep 2.0

| | | AHI ≥ 15 | AHI < 15 |
|---|---|---|---|
| Manually scored | AHI ≥ 15 | 277 | 45 |
| | AHI < 15 | 14 | 383 |

BodySleep 2.0

| | | AHI ≥ 30 | AHI < 30 |
|---|---|---|---|
| Manually scored | AHI ≥ 30 | 109 | 26 |
| | AHI < 30 | 10 | 574 |

TABLE 2-26
Confusion Matrix for BMI severity:
Overweight Obese (30 ≤ MBI)

BodySleep 2.0

| | | AHI ≥ 5 | AHI < 5 |
|---|---|---|---|
| Manually scored | AHI ≥ 5 | 889 | 23 |
| | AHI < 5 | 9 | 287 |

BodySleep 2.0

| | | AHI ≥ 15 | AHI < 15 |
|---|---|---|---|
| Manually scored | AHI ≥ 15 | 607 | 67 |
| | AHI < 15 | 16 | 518 |

BodySleep 2.0

| | | AHI ≥ 30 | AHI < 30 |
|---|---|---|---|
| Manually scored | AHI ≥ 30 | 330 | 45 |
| | AHI < 30 | 15 | 818 |

TABLE 2-27
Confusion Matrix for the age group: 18-25

BodySleep 2.0

| | | AHI ≥ 5 | AHI < 5 |
|---|---|---|---|
| Manually scored | AHI ≥ 5 | 177 | 7 |
| | AHI < 5 | 14 | 64 |

BodySleep 2.0

| | | AHI ≥ 15 | AHI < 15 |
|---|---|---|---|
| Manually scored | AHI ≥ 15 | 36 | 3 |
| | AHI < 15 | 16 | 207 |

BodySleep 2.0

| | | AHI ≥ 30 | AHI < 30 |
|---|---|---|---|
| Manually scored | AHI ≥ 30 | 6 | 3 |
| | AHI < 30 | 2 | 251 |

TABLE 2-28
Confusion Matrix the age group 26-35

BodySleep 2.0

| | | AHI ≥ 5 | AHI < 5 |
|---|---|---|---|
| Manually scored | AHI ≥ 5 | 196 | 10 |
| | AHI < 5 | 15 | 47 |

BodySleep 2.0

| | | AHI ≥ 15 | AHI < 15 |
|---|---|---|---|
| Manually scored | AHI ≥ 15 | 72 | 3 |
| | AHI < 15 | 20 | 173 |

BodySleep 2.0

| | | AHI ≥ 30 | AHI < 30 |
|---|---|---|---|
| Manually scored | AHI ≥ 30 | 33 | 2 |
| | AHI < 30 | 8 | 225 |

TABLE 2-29
Confusion Matrix for age groups: 36-45

BodySleep 2.0

| | | AHI ≥ 5 | AHI < 5 |
|---|---|---|---|
| Manually scored | AHI ≥ 5 | 292 | 10 |
| | AHI < 5 | 23 | 80 |

BodySleep 2.0

| | | AHI ≥ 15 | AHI < 15 |
|---|---|---|---|
| Manually scored | AHI ≥ 15 | 146 | 4 |
| | AHI < 15 | 20 | 235 |

BodySleep 2.0

| | | AHI ≥ 30 | AHI < 30 |
|---|---|---|---|
| Manually scored | AHI ≥ 30 | 67 | 1 |
| | AHI < 30 | 8 | 329 |

TABLE 2-30
Confusion Matrix the age group 46-55

BodySleep 2.0

| | | AHI ≥ 5 | AHI < 5 |
|---|---|---|---|
| Manually scored | AHI ≥ 5 | 378 | 8 |
| | AHI < 5 | 13 | 115 |

BodySleep 2.0

| | | AHI ≥ 15 | AHI < 15 |
|---|---|---|---|
| Manually scored | AHI ≥ 15 | 230 | 14 |
| | AHI < 15 | 38 | 232 |

BodySleep 2.0

| | | AHI ≥ 30 | AHI < 30 |
|---|---|---|---|
| Manually scored | AHI ≥ 30 | 109 | 6 |
| | AHI < 30 | 13 | 386 |

TABLE 2-31
Confusion Matrix the age group 56-65

BodySleep 2.0

| | | AHI ≥ 5 | AHI < 5 |
|---|---|---|---|
| Manually scored | AHI ≥ 5 | 322 | 3 |
| | AHI < 5 | 6 | 101 |

BodySleep 2.0

| | | AHI ≥ 15 | AHI < 15 |
|---|---|---|---|
| Manually scored | AHI ≥ 15 | 223 | 6 |
| | AHI < 15 | 24 | 179 |

BodySleep 2.0

| | | AHI ≥ 30 | AHI < 30 |
|---|---|---|---|
| Manually scored | AHI ≥ 30 | 100 | 6 |
| | AHI < 30 | 23 | 303 |

TABLE 2-32
Confusion Matrix the age group > 65

BodySleep 2.0

| | | AHI ≥ 5 | AHI < 5 |
|---|---|---|---|
| Manually scored | AHI ≥ 5 | 332 | 2 |
| | AHI < 5 | 6 | 111 |

BodySleep 2.0

| | | AHI ≥ 15 | AHI < 15 |
|---|---|---|---|
| Manually scored | AHI ≥ 15 | 248 | 7 |
| | AHI < 15 | 26 | 170 |

BodySleep 2.0

| | | AHI ≥ 30 | AHI < 30 |
|---|---|---|---|
| Manually scored | AHI ≥ 30 | 130 | 6 |
| | AHI < 30 | 26 | 289 |

TABLE 2-33
Confusion Matrices for all comorbidities (a-m)

BodySleep 2.0

| | |
|---|---|
| AHI ≥ 5 | AHI < 5 |

(a) Confusion Matrix ADHD

| | | | |
|---|---|---|---|
| Manually scored | AHI ≥ 5 | 39 | 2 |
| | AHI < 5 | 0 | 5 |

(b) Confusion Matrix anxiety

| | | | |
|---|---|---|---|
| Manually scored | AHI ≥ 5 | 39 | 2 |
| | AHI < 5 | 0 | 5 |

(c) Confusion Matrix for asthma

| | | | |
|---|---|---|---|
| Manually scored | AHI ≥ 5 | 37 | 0 |
| | AHI < 5 | 0 | 9 |

(d) Confusion Matrix for atrial fibrillation

| | | | |
|---|---|---|---|
| Manually scored | AHI ≥ 5 | 53 | 2 |
| | AHI < 5 | 0 | 4 |

(e) Confusion Matrix for diabetes

| | | | |
|---|---|---|---|
| Manually scored | AHI ≥ 5 | 48 | 2 |
| | AHI < 5 | 0 | 9 |

(f) Confusion Matrix for heart disease

| | | | |
|---|---|---|---|
| Manually scored | AHI ≥ 5 | 33 | 1 |
| | AHI < 5 | 2 | 10 |

(g) Confusion Matrix for heart failure

| | | | |
|---|---|---|---|
| Manually scored | AHI ≥ 5 | 9 | 1 |
| | AHI < 5 | 1 | 2 |

(h) Confusion Matrix for hypertension

| | | | |
|---|---|---|---|
| Manually scored | AHI ≥ 5 | 164 | 3 |
| | AHI < 5 | 4 | 46 |

(i) Confusion Matrix for low testosterone

| | | | |
|---|---|---|---|
| Manually scored | AHI ≥ 5 | 30 | 2 |
| | AHI < 5 | 0 | 12 |

(j) Confusion Matrix for obesity

| | | | |
|---|---|---|---|
| Manually scored | AHI ≥ 5 | 888 | 23 |
| | AHI < 5 | 9 | 287 |

(k) Confusion Matrix for reflux

| | | | |
|---|---|---|---|
| Manually scored | AHI ≥ 5 | 178 | 10 |
| | AHI < 5 | 4 | 62 |

(l) Confusion Matrix for seasonal

| | | | |
|---|---|---|---|
| Manually scored | AHI ≥ 5 | 53 | 1 |
| | AHI < 5 | 3 | 1 |

(m) Confusion Matrix for seizures

| | | | |
|---|---|---|---|
| Manually scored | AHI ≥ 5 | 14 | 0 |
| | AHI < 5 | 0 | 1 |

TABLE 2-34
Confusion Matrix for BDZ

BodySleep 2.0

| | | AHI ≥ 5 | AHI < 5 |
|---|---|---|---|
| Manually scored | AHI ≥ 5 | 22 | 0 |
| | AHI < 5 | 1 | 11 |

BodySleep 2.0

| | | AHI ≥ 15 | AHI < 15 |
|---|---|---|---|
| Manually scored | AHI ≥ 15 | 12 | 0 |
| | AHI < 15 | 1 | 21 |

BodySleep 2.0

| | | AHI ≥ 30 | AHI < 30 |
|---|---|---|---|
| Manually scored | AHI ≥ 30 | 6 | 0 |
| | AHI < 30 | 1 | 27 |

TABLE 2-35
Confusion Matrix for Beta-blockers

BodySleep 2.0

| | | AHI ≥ 5 | AHI < 5 |
|---|---|---|---|
| Manually scored | AHI ≥ 5 | 22 | 0 |
| | AHI < 5 | 1 | 11 |

BodySleep 2.0

| | | AHI ≥ 15 | AHI < 15 |
|---|---|---|---|
| Manually scored | AHI ≥ 15 | 24 | 0 |
| | AHI < 15 | 3 | 19 |

BodySleep 2.0

| | | AHI ≥ 30 | AHI < 30 |
|---|---|---|---|
| Manually scored | AHI ≥ 30 | 11 | 0 |
| | AHI < 30 | 2 | 33 |

TABLE 2-36
Confusion Matrix for NDRI

BodySleep 2.0

| | | AHI ≥ 5 | AHI < 5 |
|---|---|---|---|
| Manually scored | AHI ≥ 5 | 43 | 0 |
| | AHI < 5 | 2 | 18 |

BodySleep 2.0

| | | AHI ≥ 15 | AHI < 15 |
|---|---|---|---|
| Manually scored | AHI ≥ 15 | 27 | 1 |
| | AHI < 15 | 2 | 33 |

BodySleep 2.0

| | | AHI ≥ 30 | AHI < 30 |
|---|---|---|---|
| Manually scored | AHI ≥ 30 | 18 | 0 |
| | AHI < 30 | 3 | 42 |

TABLE 2-37
Confusion Matrix for Opioids

BodySleep 2.0

| | | AHI ≥ 5 | AHI < 5 |
|---|---|---|---|
| Manually scored | AHI ≥ 5 | 14 | 0 |
| | AHI < 5 | 1 | 3 |

BodySleep 2.0

| | | AHI ≥ 15 | AHI < 15 |
|---|---|---|---|
| Manually scored | AHI ≥ 15 | 9 | 0 |
| | AHI < 15 | 2 | 7 |

BodySleep 2.0

| | | AHI ≥ 30 | AHI < 30 |
|---|---|---|---|
| Manually scored | AHI ≥ 30 | 5 | 0 |
| | AHI < 30 | 0 | 13 |

TABLE 2-38
Confusion Matrix for SARI

BodySleep 2.0

| | | AHI ≥ 5 | AHI < 5 |
|---|---|---|---|
| Manually scored | AHI ≥ 5 | 38 | 2 |
| | AHI < 5 | 1 | 21 |

BodySleep 2.0

| | | AHI ≥ 15 | AHI < 15 |
|---|---|---|---|
| Manually scored | AHI ≥ 15 | 21 | 1 |
| | AHI < 15 | 2 | 38 |

BodySleep 2.0

| | | AHI ≥ 30 | AHI < 30 |
|---|---|---|---|
| Manually scored | AHI ≥ 30 | 7 | 1 |
| | AHI < 30 | 0 | 54 |

TABLE 2-39
Confusion Matrix for SNRI

BodySleep 2.0

| | | AHI ≥ 5 | AHI < 5 |
|---|---|---|---|
| Manually scored | AHI ≥ 5 | 24 | 1 |
| | AHI < 5 | 1 | 6 |

BodySleep 2.0

| | | AHI ≥ 15 | AHI < 15 |
|---|---|---|---|
| Manually scored | AHI ≥ 15 | 15 | 0 |
| | AHI < 15 | 0 | 17 |

BodySleep 2.0

| | | AHI ≥ 30 | AHI < 30 |
|---|---|---|---|
| Manually scored | AHI ≥ 30 | 8 | 0 |
| | AHI < 30 | 0 | 24 |

TABLE 2-40
Confusion Matrix for SSRI

BodySleep 2.0

| | | AHI ≥ 5 | AHI < 5 |
|---|---|---|---|
| Manually scored | AHI ≥ 5 | 83 | 3 |
| | AHI < 5 | 5 | 24 |

BodySleep 2.0

| | | AHI ≥ 15 | AHI < 15 |
|---|---|---|---|
| Manually scored | AHI ≥ 15 | 37 | 3 |
| | AHI < 15 | 7 | 68 |

BodySleep 2.0

| | | AHI ≥ 30 | AHI < 30 |
|---|---|---|---|
| Manually scored | AHI ≥ 30 | 20 | 2 |
| | AHI < 30 | 5 | 88 |

TABLE 2-41
Confusion Matrix for Stimulants

BodySleep 2.0

| | | AHI ≥ 5 | AHI < 5 |
|---|---|---|---|
| Manually scored | AHI ≥ 5 | 17 | 0 |
| | AHI < 5 | 3 | 14 |

BodySleep 2.0

| | | AHI ≥ 15 | AHI < 15 |
|---|---|---|---|
| Manually scored | AHI ≥ 15 | 6 | 0 |
| | AHI < 15 | 4 | 24 |

BodySleep 2.0

| | | AHI ≥ 30 | AHI < 30 |
|---|---|---|---|
| Manually scored | AHI ≥ 30 | 2 | 0 |
| | AHI < 30 | 1 | 31 |

### Discussion

This study aimed to validate the methods disclosed herein, for example, in a preferred embodiment of the method that is described herein as the “Nox BodySleep2.0” in classifying sleep stages and detecting arousals across the diverse subgroups of demographics, comorbid conditions, and medication types. This retrospective analysis compared AHI values, of PSG and HSAT+ studies, obtained from the automatic respiratory analysis using the algorithms scoring, to AHI values obtained from the same respiratory analysis using manual scoring. The results indicate the algorithm is a good tool compared to the reference method.

Overview of the Complete Dataset When comparing the test AHI and the reference AHI, the AHI value obtained using the Nox BodySleep2.0 demonstrated very good overall agreement, when AHI≥5, across the whole dataset. The agreement values remained high even with rising AHI values.

In the context of predictive value analysis, the algorithm demonstrated a good performance. The PPV being high overall indicates the algorithm is good compared to the reference method with regards to accurate sleep stage and arousal estimation for sleep studies with AHI≥5. The NPV being high means the algorithm is good at estimating sleep stages and arousals for sleep recordings when AHI<5. A greater threshold for AHI Showed higher values of NPV indicating a slightly better performance of The BodySleep2.0 evaluating AHI under the new threshold.

The Bland Altman plot for the entire dataset, showing data points mostly being concentrated around the zero difference line, with no obvious bias, means there is a good level of agreement between the two methods. Similar conclusions can be made by the high Cohen's Kappa coefficient and high F1 score.

Demographics The Bland Altman plots for all subgroups showed similar results as the plot for the whole dataset, indicating a good level of agreement between the Nox BodySleep2.0 and the reference method for all subgroups.

The confidence intervals for the agreement statistics showed that the Nox BodySleep2.0, in general, performs well compared to the reference method for all subgroups. The greater upper and lower bounds and smaller ranges observed in males may suggest that the algorithm performs slightly better for males compared to females. The increase of confidence interval bounds, for PPA, NPA, and OPA, with older age could mean that the Nox BodySleep2.0 performs better on recordings from individuals as they get older. In the same way, increasing the limits of all confidence intervals with a higher BMI could imply the Nox BodySleep2.0 performing slightly better with a higher BMI.

The predictive value statistics showed a similar trend. The confidence intervals had high bounds for both statistics, indicating that the Nox BodySleep2.0 is both good at estimating individuals with AHI ≥5 and AHI <5 for all subgroups. The lower confidence interval bounds for NPV could be explained by fewer individuals with AHI <5 in the dataset for all subgroups.

Comorbidities When looking at individuals with comorbidities, the CI limits were relatively high for all statistics except for the NPV for individuals with gastroesophageal reflux. Among the gastroesophageal reflux patients, the automated analysis tends to slightly underestimate AHI levels in individuals with a threshold of AHI ≥5. It is important to highlight that existing literature suggests that Gastroesophageal Reflux could potentially introduce effects that may affect measurements. Breathing patterns in individuals with gastroesophageal reflux can be distinctive and unique, which may not be accurately captured by the algorithm, likely due to insufficient training data on individuals with this condition. Consequently, less common and unique behaviors may not be adequately incorporated or considered. The few individuals in the dataset with AHI<5 and certain comorbidities may help explain some of the confidence intervals for PPA and PPV being [100, 100] and NPA and NPV being [0, 100] or [100, 100] in tables 2-12 and 14.

Medication The high bounds on all confidence intervals for all performance metrics on all subgroups imply that the performance of the BodySleep2.0 algorithm on individuals taking medications was similar across all subgroups. The overall agreement suggests that the algorithm is not likely interrupted by people taking the medications described in this study. Some of the CIs for PPA and PPV being [100, 100] and NPA and NPV [0, 100] or [100, 100] in Tables 2-12 and 2-14 may be explained by the lack of sleep studies in the dataset that involve individuals taking medication and the small number of them with AHI<5.

### Long-Term Implications and Practical Applications

The use of CNN algorithms, like the Nox BodySleep2.0 in conjunction with the HSAT studies to estimate sleep stages and score respiratory and arousal events may reduce the amount of time sleep technologists must spend on PSG scoring, allowing them to devote more time to patient needs. It could also enable experts in sleep medicine to effectively compile enormous amounts of data from various sources, including HAST studies, and could potentially lead to substantial savings in both cost and time for patients and clinicians. It also serves to solve the problem of the tedious waiting for in-lab PSG.

### CONCLUSION

This study's methodology is one of its key strengths. By extracting specific parameters from the PSG and HSAT+ data for comparison, the potential variance from differing sleep nights or double setups is eliminated. This method also allowed for comparing the algorithm's performance against the gold standard PSG manual scoring, further increasing this study's generalizability.

In conclusion, the BodySleep method as disclosed herein (i.e., the Nox BodySleep2.0) demonstrated a consistently high level of agreement in accurate AHI classification for those with AHI≥5 across all subgroups. While minor variations were observed in some demographic and comorbidity subgroups, these were minimal. The BodySleep method as disclosed herein, in the embodiment referred to as the Nox BodySleep2.0, can be used with confidence to more accurately estimate AHI severity in HSAT studies.

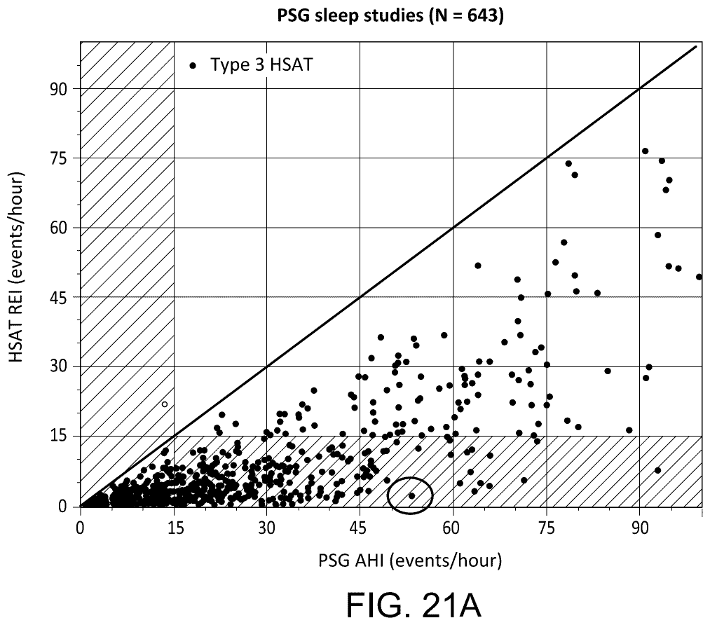

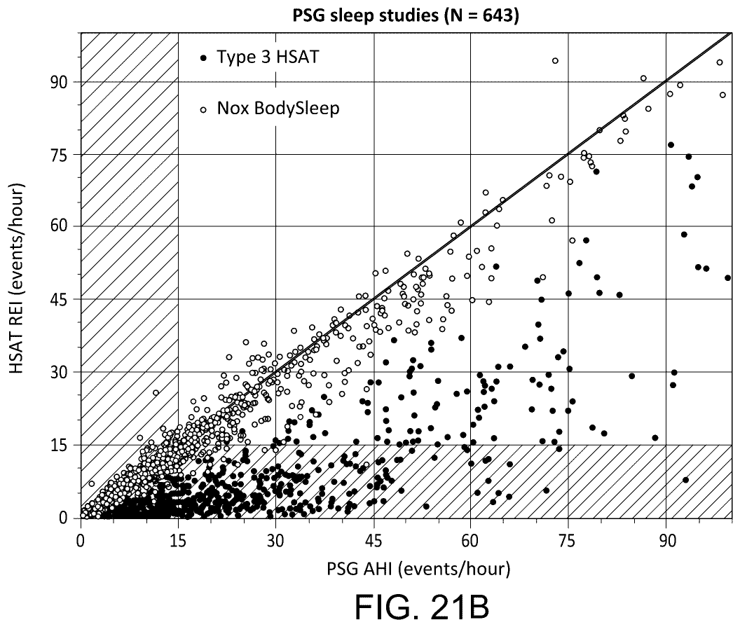

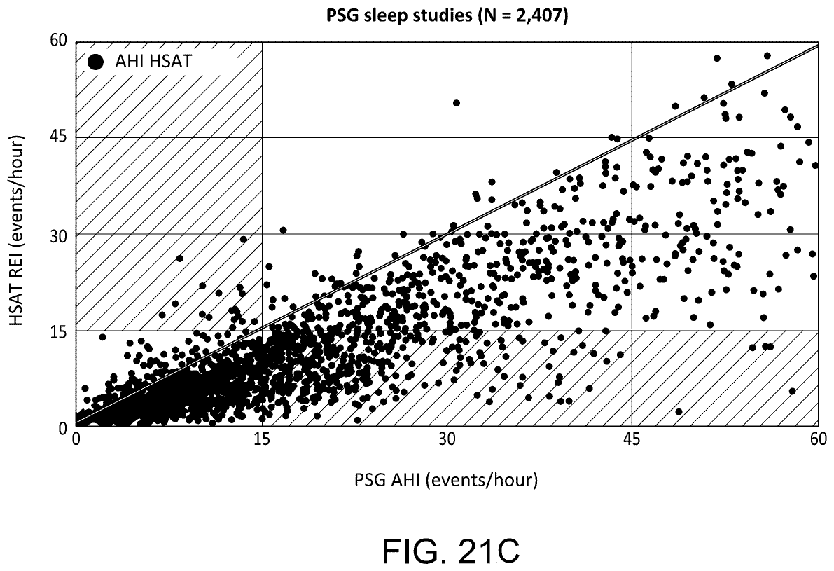

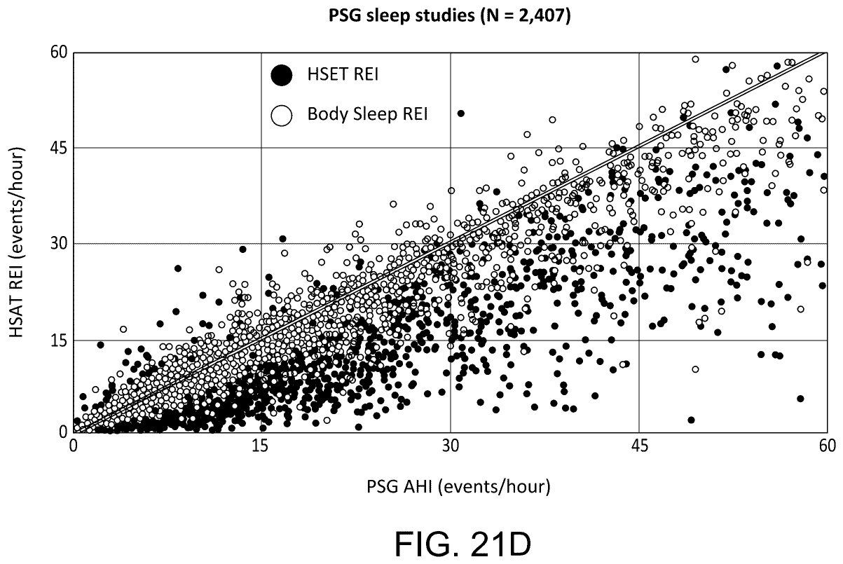

FIGS. 21A and 21C show the systematic underestimation of Home Apnea Sleep Testing (HSAT) Respiratory Event Index (REI) using current HSAT methods compared to Polysomnography (PSG) Apnea Hypopnea Index (AHI), and 21B and 21D show the significant improvement to accuracy of the Home Apnea Sleep Testing (HSAT) Respiratory Event Index (REI) using an embodiment of the non-brain, BodySleep analysis method as described herein.

In the results shown in FIGS. 21A and 21C, in comparing AHI with REI which are generally highly correlated, AHI is calculated as the count of (Apnea; Hypopnea with Desat; Hypopnea with Arousal)/Total Sleep Time, and REI is calculated as the count of (Apnea; Hypopnea with Desat)/Recording Time. It is noted that hypopneas are only counted once, even if they fulfill both the Desat and Arousal criteria. Total Sleep Time is typically measured with EEG, which is missing in the regular HSAT study. Therefore the accepted standard is to use Recording Time as the maximum approximation, which systematically drives down the REI compared with AHI.

As arousals are in the standard determined from EEG in PSG. But an EEG signal is missing in the HSAT and therefore arousals and arousal-associated events, such as arousal-associated hypopneas, so this also drives the REI systematically down compared with AHI, resulting in a significant underestimation the HSAT REI. The overall result is that HSAT REI is by definition >=AHI and this means that if the HSAT-REI-number is above the AHI-clinical thresholds for sleep apnea, the diagnosis is “conclusive” as PSG would only drive it higher.

In the same way, if it is lower, the HSAT should be considered “inconclusive” as the AHI could have been above the threshold. This means that for some patients, (especially women and kids) all the apneas/hypopneas may not meet the criteria captured by the HSAT, but all would be arousal based. This would make the HSAT deliver a REI of 0 but AHI of 50 as an example. So the correlation is 100% for Apneas and Hypopneas with Desat as they were determined in the same way on the same signals, but 0 on the arousal based.

Relating to the AHI thresholds of 5 and 15, these are the standard AHI clinical thresholds used for diagnosing sleep apnea. >5=Normal, 5-15=Mild, 15-30=Moderate and 30+=Severe. These thresholds in most clinics determine the therapy, people with chronic conditions and “mild” or above, in many programs get CPAP treatment, while others require Moderate or above to get CPAP. For those that are Mild, without chronic conditions, they receive alternative treatments from CPAP, often in the form of oral appliances. Accordingly, it is important to classify the patients correctly into those categories.

In the data shown in FIGS. 21A and 21C, a type 3 HSAT study was performed. The circled data point in FIG. 21A illustrates a patient with REI=0.8 on type 3 HSAT but a AHI=22.7 on a PSG.

The data shown in FIGS. 21B and 21D show the HSAT performed according to the disclosed method wherein arousals and arousal-associated event are determined based on body signals (in this case RIP signals from 2 RIP belts, in a study of 643 sleep studies and 2,407 sleep studies respectively.

In FIG. 21B, the patient level agreement for AHI ≥5 are shown in Table 3-1 below.

TABLE 3-1

Patient level agreement

| AHI ≥ 5 | Sensitivity | Specificity | Accuracy |
|---|---|---|---|
| No arousals | 68% | 100% | 75% |
| Noxturnal PSG analysis | 96% | 95% | 96% |
| BodySleep | 96% | 92% | 95% |

And for the results shown in FIG. 21B, the patient level agreement for AHI ≥15 are shown in Table 3-2 below.

Patient level agreement

| AHI ≥ 15 | Sensitivity | Specificity | Accuracy |
|---|---|---|---|
| No arousals | 54% | 100% | 80% |
| Noxturnal PSG analysis | 89% | 98% | 96% |
| BodySleep | 88% | 96% | 93% |

FIGS. 21C and 21D show a similar validation of 2,407 studies, when using HSAT (with no EEG signal) and without an arousal detection as described herein, 1,937 (81%) subjects get the correct outcome, comparing the measured HSAT REI with the actual PSG AHI. However, when using the body-sleep based RIP signals and the methods described herein to determine arousals and arousal-associated events, 2,211 (92%) subjects get the correct outcome and the sleep studies and REI of 274 individuals (11%) are properly and correctly assessed.

In FIG. 21D, the patient level agreement for AHI ≥5 are shown in Table 3-3 below.

TABLE 3-3

Patient level agreement

| AHI ≥ 5 | Sensitivity | Specificity | Accuracy |
|---|---|---|---|
| No arousals | 75% | 95% | 77% |
| Noxturnal PSG analysis | 96% | 95% | 96% |
| BodySleep | 95% | 88% | 94% |

In FIG. 21D, the patient level agreement for AHI ≥15 are shown in Table 3-4 below.

TABLE 3-4

Patient level agreement

| AHI ≥ 15 | Sensitivity | Specificity | Accuracy |
|---|---|---|---|
| No arousals | 60% | 99% | 81% |
| Noxturnal PSG analysis | 89% | 98% | 96% |
| BodySleep | 86% | 97% | 92% |

As used in Tables 3-1 to 3-4, “No Arousals” simply means that these are the standard HISAT results without a determination of body-signal or body-based arousal or arousal-associated events, or in other words that is not counting the arousal based hypopneas. The Afib—is a shortcut for Afibrillation that is a common cardiac condition. So the Afib dataset consists of patients undergoing a sleep study that have additionally this Afib-condition (Irregular heartbeat). This shows a significant advantage of the body-signal (and particularly RIP belt) based method described herein, as compared to the pulse-oximeter measurements (or more accurately photoplethysmography, PPG). Comparing a “respiratory based sleep studies” as described herein and “PPG based sleep studies”, a significant difference is that PPG based sleep studies struggle with accuracy if the patients have cardiac conditions, such as Afib, but the Respiratory Based accuracy is not affected like can be seen from the tables above.

So to have a realistic comparison for the performance of the RIP based signals to determine arousals and arousal-associated events, the PSG were auto-scored, with an AI model including all the PSG-EEG, EMG and ECG signals as well as the HSAT signals. So in Tables 3-1 to 3-4, are shown 1. Standard auto scored HSAT, 2. Standard auto stored PSG (with AI model reading the EEG channels) and 3. HSAT including RIP-based determination of arousals and arousal-associated events as described herein. All compared favorably with the “golden standard” of the manually scored PSG study.

This was unexpected and surprising, when the results are considered, as it demonstrates that the information that matters for AHI scoring are all found in the HSAT signals, including the RIP belt signals, and the EEG does not add anything of importance to the accuracy.

Is based on the standard HSAT signals but provides AHI closely matching what would be expected from PSG. It archives this by, in addition to performing standard REI calculation, correctly detects the normally missing arousal based hypopneas and determines accurate Total Sleep Time.

Its accuracy is in-significantly affected by cardiac conditions, such as Afib and therefore is a big-improvement when compared with cardiac/pulse based products (such as PAT) suffering from big contraindications for all types of cardiac conditions.

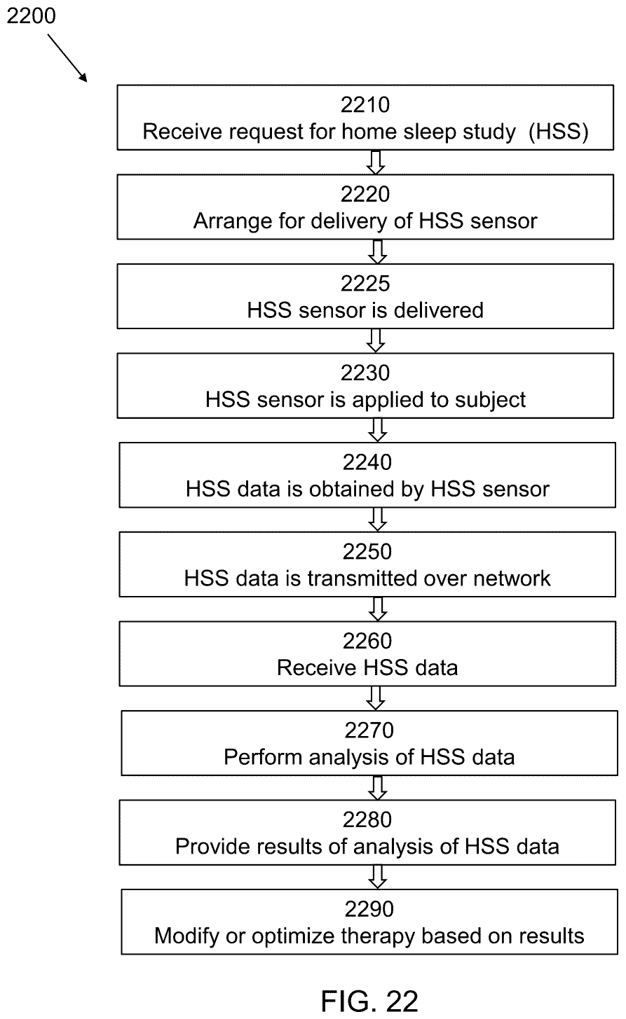

According to an example as shown in FIG. 22, the systems, sensors, and methods described herein (e.g., as implemented in a Nox BodySleep2.0) provide a method 2200 of obtaining results from a home sleep study (HSS) that are significantly improved to level of accuracy similar to a PSG study. In step 2210, a request is received for a home sleep study (HSS). Such a request may be received over the Internet or through a network. At step 2220, delivery of a HHS sensor is arranged. At step 2225, the HHS sensor is delivered to the subject. This may be through a common carrier or delivery, often to the home of the subject. At step 2230, the HHS sensor is applied to the subject. In a preferred example, the HHS sensor includes a RIP belt sensor system including one or more, preferably two, RIP belts, including a thoracis RIP belt and an abdomen RIP belt, as shown, for example, in one of FIGS. 7A-7C. During one or more nights, HSS data is obtained by the HHS sensor. This data may be stored by the HHS sensor, or may be transmitted to a central server or computing device through a Bluetooth transmission, LAN, or cellular network, or the Internet. Or, the HHS sensor or a data storage device can be mailed, shipped, or delivered by carrier to a remote location housing the central server or computer device. Using the body-signal based system and methods described herein, a BodySleep is implemented including determining an arousal of the subject using the data from one or more body signals. A result is provided based on the RIP data including the determined arousals. And certain diagnosis can be made, a sleep therapy can be prescribed or implemented, a therapy can be modified based on the BodySleep analysis results. The method of 2200 provides for a high-quality HSS with results very comparable to a PSG without requiring clinical involvement in delivery or operation of the HSS. The HSS can be performed entirely by the subject and the HSS data can be uploaded to the Cloud or Internet, and can be analyzed and the results can be provided to the subject, for example, in a web accessible platform. And based on the results, a therapy can be recommended, begun, prescribed, of modified to improve the sleep of the subject.

Further elements may also be included in the method 2200 shown in FIG. 22 or a similar method. The device shipped or delivered to the subject may include a recorder with one or more or preferably two RIP belts. The device delivered may also or alternatively include an accelerometer, or could be two devices, one on each RIP belt and a separate acceleration meter OR only two belts. The RIP belts may be reusable for multiple nights. The device may come with a Mobile Application or other “Receiving Unit”, that has a wireless connection to the Device (Devices) for receiving the recorded RIP signals and acceleration signals (if included) in real time OR delayed during the night OR upload after the night. The Mobile Application may establish a link to the Cloud or over the Internet, for uploading the recorded data OR the Mobile Application run the BodySleep analysis on the data OR the “Receiving Unit” may upload or perform the BodySleep analysis. Based on the recorded data, a set of sleep parameters are derived, such as sleep profile, arousals, sleep disordered breathing parameters, arousal index, and/or sleep stability measures. The device may be wireless and rechargeable by the patient. The device may be activated when the RIP belts are snapped around the patient and deactivated after removal from the patient. The HSS data from the recording may be presented to a person operating in patient care management and/or to the patient himself. The data from the recording may be used to determine if a sleep therapy is effective, needs adjustment and guide how to adjust the therapy. The results from the processing may be presented to a healthcare professional for diagnosis of a sleep disorder, confirmation of health sleep or monitoring of treatment performance.

Further to the concepts, embodiments, systems, and validations of those concepts, embodiments, and systems, the following provides further, significant real-life applications and/or uses of the method and systems described therein.

A new type of processing signals from HSAT devices.

As described above, the RIP signals and optionally an acceleration signal is received and analysis according to the BodySleep analysis methods described herein. An arousal and sleep profile can be provided as additional parameters for scoring a HSAT recording. An accurate measure of standard Sleep Disordered Breathing condition can be provided that is compared to AASM PSG performance. Alternative parameters can be provided from the HSAT study such as endotypes that would normally require PSG. Results in a conclusive diagnosis of SDB from HSAT that would normally require PSG recording.

### Quality Assurance of HSAT Using BodySleep Analysis Methods

Instead of relying on using BodySleep analysis methods described herein for diagnoses, it can be used for qualifying HSAT recordings. A normal HSAT diagnosis may be performed, which as was the state of the art before the filing of the present disclosure, hypopneas and/or arousals are missed. The BodySleep analysis automation is performed and compared with the HSAT. In case of significant difference the study is determined as inconclusive and the patient is sent to PSG for a conclusive measurement.

A New Type of Processing Signals from PSG

Same as above for HSAT analysis, but the results of the BodySleep analysis methods work as surrogate signals for standard PSG signals, such as if the EEG signals are of bad quality etc. This application would be especially important for Home Sleep PSG testing (Type II or Self-Applied-Somnography) as there may be considerable failure rate for hookup done by the patient that can then be mitigated by using the BodySleep analysis output. In case that measure, such as PSG/SAS does not provide the required EEG and EOG signals, the BodySleep can be used instead. In case that there is a great difference between the PSG/SAS and BodySleep results, the study can be deemed inconclusive and may need to be repeated.

### Managing Treatment of Sleep Disorders

Introduction: Sleep Apnea. Sleep care management is significant and growing field and for good reason. Compliance to therapy, especially Continuous Positive Airway Pressure (CPAP), is very low in an unmanaged model, as it takes training and adaptation to get the therapy right and the patient comfortable. Unmanaged therapy has demonstrated over 50% non-compliance in the first year in multiple studies over the years. A managed therapy has however been demonstrated by the Applicant and the inventors to over 90% adherence, thanks to continuous monitoring on performance over radio modules in the CPAP's and high-quality service by care managers. Similar to having a personal trainer for exercise, the care-managers use a preemptive approach to spot patients struggling and adjust their therapy to be effective again. This model, however, only supports CPAP units with a radio module, but alternative therapies such as oral appliances therapy (OAT), do not support this option. Therefore, OAT is rarely used in value-based programs, as they are hard to monitor. Using a simple Respiratory Inductance Plethysmography (RIP) belt sensor device that the patient can use to measure one or more nights once or more often during the year, would be sufficient to both help the patient to adjust to alternative therapies like OAT and regularly check the patient's compliance. Other alternative therapies include hypoglossal nerve stimulation by an implantable device, pharmacological treatments, surgeries, positional treatment, lifestyle interventions, or any other treatments.

With this issue of OAT and other alternative therapies being a fact, more and more solutions are surfacing indicated with the potential of being used for this purpose. Commercial products, such as Apple watches or Oura ring have the potential to provide this type of information if they were somehow calibrated to an accurate reference as is described in Applicant's earlier application directed to calibrating SSS with an HASS in Applicant's earlier patent application U.S. application Ser. No. 17/351,933, filed at the USPTO on Jun. 18, 2021, entitled “PERSONALIZED SLEEP CLASSIFYING METHODS AND SYSTEMS”, which is herein incorporated by reference. This process, of using the methods and systems described herein, and particularly the Nox BodySleep2.0 analyzed data from a simple sensor device with 2 RIP belts (preferable single patient use) would both provide a high accuracy sleep study information from a simple and low-cost screener that could secondly by used in combination with a wearable, to calibrate it (according to U.S. application Ser. No. 17/351,933).

The methods and systems described herein also support the monitoring of CPAP use and is then agnostic or unconcerned to the CPAP device, brand, model, and method of application. In cases where a patient suffers mask leakage or is given a nasal mask, yet breathes with the mouth open, the RIP belts will accurately measure the breathing whereas the CPAP device may not.

Sleep care management using CPAP devices is based on data measured by the CPAP and delivered over radio modules to the cloud. The CPAP is, however, only in an indirect connection to the patient, the CPAP only “sees” the patient through the air-tube between the CPAP and the patient, based on the air-pressure and air-flow signals. Even if this gives the PAP device significant information on how well the patient is breathing, it cannot provide much information regarding how well the patient sleeps. This is especially difficult, as a patient on an inefficient treatment might not have direct apneas, but is half-cured, meaning that he might be struggling and having arousals. Similar to the concepts described in the above-noted patent applications where we used the high accuracy sleep study recording to calibrate a simplified sleep study (such as for wearable, see U.S. application Ser. No. 17/351,933) providing a clinically useful pre- and post-data analysis of the SSS, the same can be done using a sensor device as described herein that implements (e.g., the Nox BodySleep2.0 sensor) in association with the CPAP data. A recording of a few night using a sensor device as described herein (e.g., a Nox BodySleep2.0 sensor) during CPAP treatment, both provides the information how well the patient slept during those nights on treatment, but also information how to interpret the data from the CPAP up to the point of the BodySleep2.0 sensor recording and from that time one. This could improve the reliability of the data that the care managers have to work with and provide valuable and early warning if the patient's performance starts to degrade, that would eventually lead to the patient stopping using the therapy.

The systems, sensors, and methods described herein (e.g., as implemented in a Nox BodySleep2.0) can be a fundamental building block in an inexpensive sensor device (such as a 2-belt RIP sensor device) that can be used by patients to monitor the performance of the treatment they receive for their sleep disorders. The device can be shipped to the patient at some point in time and the patient can use the device with their treatment in their own home. The value of the systems, sensors, and methods described herein (e.g., as implemented in a Nox BodySleep2.0) brings to the 2-belt sensor is that it allows the 2 belt sensor to detect sleep stages and arousals in the patient along with changes in breathing, such as apneas and hypopneas. Arousal scoring is particularly important since it allows the detection of hypopneas without requiring a measurement of drops in blood oxygen saturation (SpO2). The measurement of the SpO2 signal is measured by a pulse oximeter, which is a relatively expensive device, adds complexity to the sleep study, is prone to failure, and decreases comfort.

The systems, sensors, and methods described herein (e.g., as implemented in a Nox BodySleep2.0) in conjunction with the RIP belt sensor (e.g. a 2-belt RIP system) is an inexpensive, scalable, comfortable, and accurate method to measure sleep, arousals, and sleep apnea in patients.

According, one method may include the following: At least a 2 RIP belt sensor (which may preferably be a single patient use), is sent to a customer on request. The patient downloads an app in his mobile phone that is paired with the sensor for wireless communications (such as BLE). The patient sleeps with the sensor that streams the recorded data either directly or buffered to the mobile device, where it is received and buffered before being streamed to the cloud for storage. The received data is processed by BodySleep2.0, and the analysis output provided to an adequate person responsible for monitoring the patient sleep performance or treatment compliance.

According to another method, the following same method as above is followed except the device is a recorder (for example, such as a Nox T3s recorder), that stores the data, the data is then uploaded during or after the study to the cloud OR the data on the device is downloaded upon its physical return to the clinic/operation where BodySleep2.0 is used for analyzing like in Method 1. The patient does not need to download an app.

### Calibrating a Simple Sleep Study Device

According to a calibrating method: A patient uses a consumer grade or a simple medical device to monitor his treatment effectiveness. A RIP belt sensor (preferably a 2-belt sensor and for single patient use), is sent to a customer on request. The patient downloads an app in his mobile phone that is paired with the sensor for wireless communications (such as BLE). The patient sleeps with the sensor that streams the recorded data either directly or buffered to the mobile device, where it is received and buffered before being streamed to the cloud for storage. The received data is processed by a BodySleep processor, for example, the Nox BodySleep2.0, and the analysis output is used to calibrate the consumer grade or simple medical device as described in U.S. application Ser. No. 17/351,933.

According to another method, the following same method as above is followed except the device is a recorder (like T3s), that stores the data, the data is then uploaded during or after the study to the cloud OR the data on the device is downloaded upon its physical return to the clinic/operation where BodySleep2.0 is used for analyzing like in Method 1. The patient does not need to download an app.

### Augmenting CPAP Data for Improved Therapy Management

According to another example of a method, the following is performed. First, a BodySleep method is performed using a BodySleep sensor, such as a Nox BodySleep2.0 Sensor, which records a patient using CPAP, preferable over multiple nights. A BodySleep analysis is performed, for example, according to the BodySleep2.0 sleep analysis. A comparison between the received data from the CPAP and the measured data from BodySleep2.0 is used to augment the accuracy of the CPAP data as described in WN Ref. No U.S. application Ser. No. 17/351,933. A post-data analysis is performed on stored CPAP data to determine how the patient has been trending. The calibrated CPAP data model may be used in the future to keep trending the patient with high accuracy and to identify when he is at risk of quitting therapy.

Using one or more RIP belts (for example, two RIP belts) to diagnose sleep apnea.

As described herein, sleep apnea is a disease where a patient periodically stops breathing (apnea) or has severely reduced airflow which terminates in a drop in the blood oxygen saturation or an arousal (hypopnea). Apneas and hypopneas occur during sleep. Today sleep apnea is diagnosed by measuring breathing, and blood oxygen saturation. In some cases electroencephalography (EEG), electrooculography (EOG), and electromyography (EMG) are also used. The reduction in airflow is typically measured by a nasal cannula, the desaturation is measured by an oximeter, the arousals are detected in the EEG signals, and various sleep stages are detected using the EEG, EOG, and EMG signals.

According to the American Academy of Sleep Medicine (AASM) hypopneas and apneas are defined as certain reduction in breathing followed by a drop in blood oxygen saturation or an arousal. Even though a drop in blood oxygen saturation is often used to score hypopneas, it has been shown that most respiratory events terminate in arousals.

By measuring the respiratory movements of the thorax and abdomen using RIP belts and employing a BodySleep method as described herein, it is possible to detect apneas and hypopneas from the flow signal derived from the RIP signals, a majority of the apneas and hypopneas result in an arousal which can be detected by a BodySleep method as described herein, and the sleep stages may also be detected by the BodySleep methods as described herein.

The systems, sensors, and methods described herein (e.g., as implemented in a Nox BodySleep2.0) is a foundation that can be used to design and build a new and novel device to diagnose sleep apnea and monitor sleep apnea treatment. The device is a small electronic device using two RIP belts, one placed around the thorax to measure thoracic breathing motions, and another one placed around the abdomen to measure abdomen breathing movements. The device is easy to use by the patient, can be easily shipped in the mail, is inexpensive so it is not costly if devices get lost or delayed, the devices can be disposable, the devices can live with the patient, and the devices can be used once or multiple times by the patient.

A method is provided based on the systems, sensors, and methods described herein (e.g., as implemented in a Nox BodySleep2.0) for diagnosing sleep apnea. The method comprises sending at least a 2-belt RIP sensor (which may be single patient use belts) to a customer on request. The patient downloads an app in his mobile phone that is paired with the sensor for wireless communications (such as BLE). The patient sleeps with the sensor that streams the recorded data either directly or buffered to the mobile device, where it is received and buffered before being streamed to the cloud for storage. The received data is processed by a system configured to perform the BodySleep analysis described herein (for example, the Nox BodySleep2.0), and the analysis output provided to an adequate person responsible for providing the patient with the correct diagnosis of sleep apnea.

A second is also provided, similar to the method above, except the device is a recorder (like a Nox T3 device), that stores the data, the data is then uploaded during or after the study to the cloud or the data on the device is downloaded upon its physical return to the clinic/operation where BodySleep2.0 is used for analyzing like in Method 1. The patient does not need to download an app.

Diagnosis of Sleep Disorders Others than Sleep Apnea

### Introduction: Periodic Limb Movements of Sleep (PLMS).

Patients suffering from PLMS have characteristic periodic movements or twitches of muscles in the legs or arms. PLMS is typically diagnosed by a polysomnography (PSG) sleep study where electroencephalography (EEG) and electromyography (EMG) are recorded. The EMG signals are recorded on the limbs to detect characteristic increases in muscle tone associated with the muscle twitching. The EEG signals are recorded to detect arousals associated with muscle twitching. The PLMS events have characteristic periodicity that are reflected in the EMG and arousal events. Furthermore, the arousals associated with the PLMS events are not associated with other causes of arousals that may be periodic such as breathing cessation during sleep (i.e. apneas and hypopneas).

With PLMS being one of the 3 most common sleep disorders, it is important to have a scalable method for measuring it and monitoring treatment of PLMS for titration of the medicine used. Conventionally PLMS is measured in PSG using EMG electrodes on the right and left leg and when the muscle is active, it shows up in the EMG. In PLMS (Periodic Limb Movement during Sleep) the muscular activity is periodic and must fulfill a certain criterion to be distinguishable from movements caused by arousals from sleep apnea. Typically, the period of PLMS is 20-30 seconds but can be longer, and that overlaps with the frequency of severe OSA. PLMS does not always cause cortex arousal as defined in the sleep scoring manual, but if it doesn't affect the sleep pattern it is not a problem. While the interscorer reliability of EEG manually scored arousals is low (60%) we assume that the auto scored BodySleep2.0 arousals are both sensitive and specific to real events taking place in the body. And if only PLMS activities that cause arousals matter, the PLMS can be confirmed by the following method.

Some attempts have been made to score PLMS from body signals, especially Pleth from oximetry or PAT devices. It doesn't make much sense in PSG not to use the LM channels, such as by using the scored arousals from EEG, as the arousal scoring is quite unreliable. With the systems, sensors, and methods described herein (e.g., as implemented in a Nox BodySleep2.0 system) arousal detection is likely to change so that the arousal events are reliably marked that are not associated with OSA and can therefore include this important disorder to the mix where a BodySleep analysis (for example, Nox's BodySleep2.0) is beneficial.

In a device that contains at least one and preferably at least two RIP belts where an analysis such as the Nox BodySleep2.0 is used to determine periods of wake, REM sleep, and non-REM sleep, and detect arousal events it is possible to detect arousals during different stages of sleep. Furthermore, the two RIP belts can be used to detect apneas and hypopneas since the RIP belts measure the breathing movements of a patient. The breathing movements can be used to construct a signal that is proportional to flow during breathing. The RIP flow signal can be used to detect apneas and hypopneas. Combining the sleep, arousal, and respiratory analysis applied to the RIP signals it is possible to identify arousals that are not associated with respiratory events, occur during sleep, and are periodic. These arousals can be determined to be associated with PLMS and are the foundation of using the RIP belts and the BodySleep analysis method to diagnose PLMS, instead of requiring a full PSG sleep study.

The sensitivity of the systems, sensors, and methods described herein (e.g., as implemented in a Nox BodySleep2.0 system) is preferably trained to match the PSG scoring of arousals as well as possible. For PLMS analysis the arousal model may be used and then the periodicity may be used to determine if the events detected originated from PLMS or something else. The detection of arousal and arousal-associated events can be used to capture “switching between autonomic and somatic respiratory control” without requiring a capturing of the respiratory recovery breath response in case of sleep apnea. Thus by only capturing “lower recovery breaths” with higher sensitivity where the reality is that this is of a different physiological origin. The model may be used For PLMS analysis as the arousal model is not only capturing the recovery-breath characteristics associated with sleep apneas but as well the change of respiratory control between the autonomic and somatic systems when the patient is aroused. With the arousals detected, then the periodicity may be used to determine if the events detected originated from PLMS or something else.

Using the systems, sensors, and methods described herein (e.g., as implemented in a Nox BodySleep2.0 system), a method is provided for diagnosing PLMS. At least a 2 RIP belt sensor (preferably single patient use), is sent to a customer on request. The patient downloads an app in his mobile phone that is paired with the sensor for wireless communications (such as BLE). The patient sleeps with the sensor that streams the recorded data either directly or buffered to the mobile device, where it is received and buffered before being streamed to the cloud for storage. The received data is processed the systems, sensors, and/or methods described herein (e.g., as implemented in a Nox BodySleep2.0 system). Arousals and respiratory events are detected. Arousals associated with apneas or hypopneas are excluded and the periodicity of the remaining arousals is analyzed. It is determined if a series of arousals are periodic, repeating within a regular period (allowing certain variance) as is used for scoring PLMS EEG signals in PSG. A PLMS period is marked over the time where PLMS is repeating and mark the periodic arousal events within that period as LM. The analysis output is provided to an adequate person responsible for providing the patient with the correct diagnosis of PLMS. The indexes are reported as they were scored from PSG.

A second PLMS diagnosing method is also provided, which is same as above-described method except the device is a recorder (like a Nox T3s), that stores the data. The data is then uploaded during or after the study to the cloud or the data on the device is downloaded upon its physical return to the clinic/operation where the systems, sensors, and methods described herein (e.g., as implemented in a Nox BodySleep2.0 system) are used for analyzing like in the above method. The patient does not need to download an app.

A third PLMS method is provided, similar to the first PLMS method above, except arousals and sleep stages are determined using EEG/EOG/chin EMG as per a standard PSG sleep recording. The arousals and sleep stages are used as described in the first PLMS method above when they were derived using the analysis provided by the systems, sensors, and methods described herein (e.g., as implemented in a Nox BodySleep2.0 system).

### Introduction: Diagnosis of Narcolepsy

Patients suffering from Narcolepsy are known to have cataplexy, where they lose muscle tone in the body. They are also known to transition from wake to rapid-eye movement (REM) sleep, which is a characteristic of the disease. REM sleep is characterized with the paralysis of the skeletal muscles, including but not limited to the intercostal muscles of the thorax.

Today Narcolepsy is diagnosed in several ways. One method of diagnosing Narcolepsy is to use a special sleep study protocol called Multiple Sleep Latency Test (MSLT) or a Maintenance of Wakefulness Test (MWT). Both the MSLT and MWT sleep tests require a patient to spend a night at a hospital where an in-lab polysomnography (PSG) sleep study is performed on them. The following day the patient continues to wear the PSG sleep recording device and follows a strict protocol while being monitored by a sleep technician or a nurse. These sleep studies are uncomfortable for the patient and require extensive hospital resources.

The systems, sensors, and methods described herein (e.g., as implemented in a Nox BodySleep2.0 system) is a sleep stage and arousal detection AI analysis which uses the breathing movements of the thorax and abdomen to estimate Wake, REM, and Non-REM sleep periods. During REM sleep and during cataplexy the skeletal muscles are paralyzed while the diaphragm is active. This has a significant impact on how the breathing movements of the thorax and abdomen look like. By carefully measuring the thorax and abdomen breathing movements using the highly sensitive respiratory inductance plethysmography (RIP) sensors it is possible to distinguish periods of thorax paralysis even by eye.

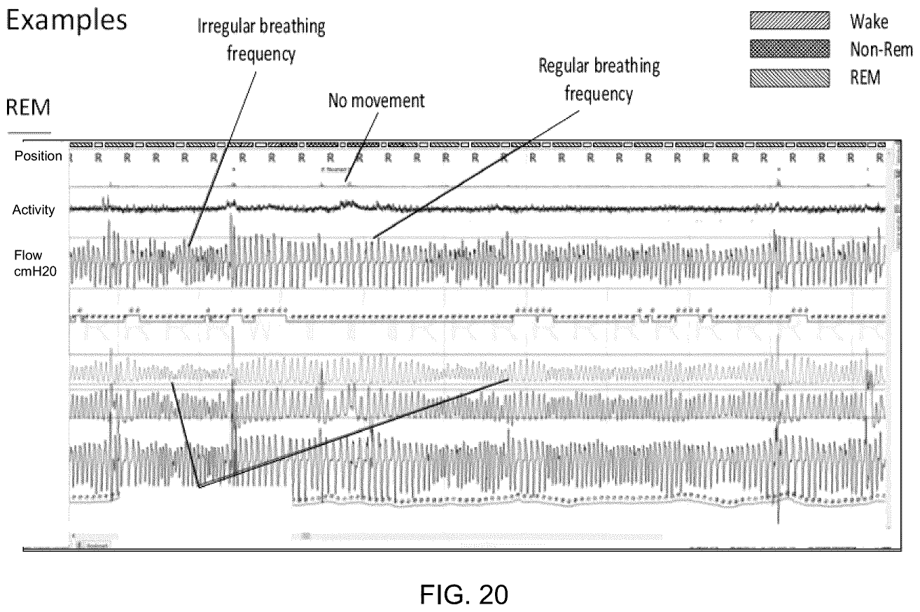

The FIG. 20 below shows an example of how the thoracic RIP signal changes in a period of REM sleep interrupted by an awakening and a period of non-REM sleep. At the top of the figure the colored markers indicate 30 second periods that have been scored as a certain sleep stage by a trained sleep technician using electroencephalogram (EEG), electrooculogram (EOG), and electromyogram (EMG) signals. The scoring of sleep stages by a sleep technician using the EEG/EOG/EMG signals is the state-of-the-art approach to score sleep. These sleep stages are presented as a reference to highlight how the abdomen and thorax RIP signals behave during REM, Wake, and Non-REM. FIG. 20 shows that the amplitude of the thoracic RIP signal is lower during the REM periods than during the Wake and non-REM periods. Furthermore, the amplitude of the thoracic signal is variable. This is indicative of intercostal muscle paralyzation.

In addition to the ability to discriminate between periods where the skeletal muscles are paralyzed a combination of sleep staging using the EEG/EOG/EMG and the analysis of the systems, sensors, and methods described herein (e.g., as implemented in a Nox BodySleep2.0 system) may be useful to detect periods of REM sleep or periods where there are discrepancies between the state-of-the-art EEG/EOG/EMG sleep staging and the sleep staging of the BodySleep analysis. It is known that narcolepsy is characterized by sleep periods where sleep technicians struggle to identify the sleep stages from the EEG/EOG/EMG signals and the patient may describe periods of where they are aware but still cannot move or control their thoughts.

A method of diagnosing Narcolepsy is provided, including using the systems, sensors, and methods described herein (e.g., as implemented in a Nox BodySleep2.0 system) to detect the transitions from wake to REM which could be used to provide input in the diagnosis of Narcolepsy. The BodySleep analysis can be used to detect cataplexy or loss of muscle tone in the body. The systems, sensors, and methods described herein (e.g., as implemented in a Nox BodySleep2.0 system), in conjunction with an analysis of the EEG/EOG/EMG signals, can be used to provide input in the diagnosis of Narcolepsy.

### Introduction: Dementia Detection

Dementia is an umbrella term used for general decline in cognitive abilities that impacts a person's ability to perform everyday activities. Dementia includes causes such as Alzheimer's disease, Lewy Body Dementia, Parkinson's Disease, and other causes. Dementia has been shown to correlate with sleep disorders and sleep disorders may even accelerate the onset of Dementia. Specific sleep disorders are known to have predictive power when detecting or diagnosing certain types of dementia.

REM sleep behavior disorder (RBD) is a core feature in the diagnosis of Lewy Body Dementia. RBD is characterized by the patient losing muscle paralysis (atonia) during REM sleep. RBD may appear years or decades before other symptoms of Lewy Body Dementia. A sleep recording device consisting of one or more RIP belts, preferably at least two RIP belts, using the systems, sensors, and methods described herein (e.g., as implemented in a Nox BodySleep2.0 system) may be used to diagnose RBD. The BodySleep methods described can distinguish between wake and sleep and can distinguish between REM and non-REM sleep. Furthermore, when the RIP belts are placed on the patient's thorax and abdomen it may be possible to detect periods when the skeletal muscles are paralyzed during REM sleep by monitoring how the thoracic breathing movements are affected. However the abdomen breathing movements are driven by the diaphragm that is not affected by the skeletal muscle paralysis during REM sleep. In Parkinson's Disease a “Meta-analyses revealed significant reductions in total sleep time, sleep efficiency, N2 percentage, slow wave sleep, rapid eye movement sleep (REM) percentage, and increases in wake time after sleep onset, N1 percentage, REM latency, apnea hypopnea index, and periodic limb movement index in PD patients compared with controls.”

A method of detecting dementia is provided. Using a device having one or more RIP belts, preferably two RIP belts and the systems, sensors, and methods described herein (e.g., as implemented in a Nox BodySleep2.0 system), RBD is detected by identifying periods of REM sleep where the patient does not have atonia. Using a device having one or more RIP belts, preferably at least two RIP belts, and the BodySleep analysis method described herein, PLMS is detected using the methods described in the section Diagnosing PLMS.

According to another method, using a device having one or more RIP belts, preferably at least two RIP belts and a BodySleep analysis method as described herein, any of the parameters mentioned in the Meta analysis including total sleep time, sleep efficiency, REM sleep percentage, increase in wake after sleep onset, REM latency, apnea hypopnea index, and PLMS.

### Augmenting Current State-of-the-Art in Sleep Diagnostics

### Introduction: HSAT Augmentation

A system, sensor, and methods described herein (e.g., as implemented in a Nox BodySleep2.0 system) is a massive improvement for HSAT studies as it allows the correct detection of all the AHI events, hypopneas that do not end in desaturations but only arousals, and provides a classification of sleep stages into WAKE/NREM/REM. Even if this provides a conclusive sleep apnea study regardless of the AHI index we do not have a reliable way of providing the same accuracy of sleep profiles as received from an in-lab PSG. An important outstanding part is the classification of NREM into N1, N2 and N3 that is important for determining sleep stability and neurologic sleep disorders. With the information already in place from HSAT with a BodySleep analysis method as described herein, it doesn't take much to augment the information to complete the PSG picture. The inventors have demonstrated from our SelfAppliedSomnography (SAS) recordings for A1s (PSG) that the frontal EEG signals (recorded on the forehead) do contain everything that is needed to derive a PSG equivalent sleep staging accuracy. It is therefore possible to record one or two EEG signals using the a HSAT device, such as a Nox T3s device, in addition to the regular HSAT signals (the T3s already has those two channels and when this is done, we call it HSAT+) and score the NREM periods into N1, N2 and N3 based on the EEGs.

HSAT+ has been used to score arousals and sleep stages before. However, combining such processing on the EEG with the results from BodySleep2.0, both providing accurate timing of the NREM periods and all the arousals, to both get the most accurate classification of sleep during NREM and improve the accuracy of the overall sleep staging and arousals, is something new.

A method is provide for augmenting HSAT with the systems, sensors, and methods described herein (e.g., as implemented in a Nox BodySleep2.0 system). According to a first method, a recording device, such as a Nox T3s device is used to record a HSAT study with one or two additional frontal-EEG channels and one or more RIP belts, preferably at least 2 RIP belts. A BodySleep system, sensors, or method as described herein (e.g., as implemented in a Nox BodySleep2.0 system) is used to derive all the parameters that can be derived from the BodySleep signals. An auto scoring is run on the EEG signals, determining sleep stages and arousals. The results are combined from both in a relative manner for highest accuracy PSG like outcomes, including PLMS from above, sleep staging, sleep time, and combined/optimal arousals.

### Introduction: Body Position and Activity Using RIP Signals

Most sleep studies contain activity and body position sensors to report along with the recorded sleep data. This is normally done using acceleration sensors that are now relatively low cost and easy to implement in any product. The body position is used to classify the apnea event based on body position, as if the apneas are for example only occurring when the patient is in supine position but not on the right or left sides, alternative treatment like oral appliance or even “tennis balls on the back” can be sufficient to avoid the apnea. For a disposable product, the acceleration sensors are however both relatively expensive with the total cost of the device and consume power that enlarges the battery needed for the same recording time. It would therefore be beneficial if the sensor could be removed but the position and activity information could be derived in a different way. The RIP belts are indeed a movement sensor that is affected by any body motion that affects the form of the abdomen or the thorax. Additionally, the alignment of the organs changes with the different body positions, and this affects the movements of the thorax towards the abdomen. Upright position means that the abdomen pressure is taken off the diaphragm and therefore the thorax compartment and this shows up in the RIP signals. Supine means that the rib-cage is free from the arms to move up and down, while left and right positions are hindered by the pressure on the side, especially if an arm is pressing the side. Prone position presses on both compartments.

Below, in Table 4-1, the validation and performance of a system and method as described above are provided wherein a 2-belt RIP system was used to determine the body position of the subject, comparing the predicted label of the body position to the true label of the body position, with an accuracy of 0.82 for the non-supine body position, 0.98 for the upright body position, and 0.82 for the supine body position. Thus, showing that the body position of the subject can be accurately or acceptably accurately determined based only on the signals of 2 RIP belts, thus simplifying the devices required and improving the accuracy of the HSAT sleep study.

TABLE 4-1

| | | Predicted label | | | |
|---|---|---|---|---|---|
| | | Non-Supine | Upright | Supine | Total |
| True | Non-Supine | 182,945 | 3,462 | 29,086 | 215,493 |
| Label | Upright | 2,436 | 15,102 | 855 | 18,393 |
| | Supine | 55,739 | 4,390 | 202,700 | 262,829 |

| | Sensitivity | Specificity | Accuracy |
|---|---|---|---|
| Non-Supine | 0.85 | 0.79 | 0.82 |
| Upright | 0.82 | 0.98 | 0.98 |
| Supine | 0.77 | 0.87 | 0.82 |

A method of determining a body position and activity is provided using RIP signals. According to a first method, so, it is clear that the information of the body position are in the RIP signals and extracting them with an AI model trained towards a standard body position signals recorded in all sleep devices can be done. Activity is the same, any body movement that is large enough to move the abdomen or thorax, shows up as irregularity in the belts and can be marked with an AI model trained on the activity data from a sleep study.

### Augmenting HSAT Studies to Contain Full PSG Information

Background The systems, sensors, and methods described herein (e.g., as implemented in a Nox BodySleep2.0) are a massive improvement for HSAT studies as it allows the correct detection of all the AHI events and provides a classification of sleep stages into WAKE/NREM/REM. Even if this provides a conclusive sleep apnea study regardless of the AHI index we do not have a reliable way of providing the same accuracy sleep profiles as received from an in-lab PSG. The most important outstanding part is the classification of NREM into N1, N2 and N3 that is important for determining sleep stability and neurologic sleep disorders. With the information already in place from HSAT with BS2, it doesn't take much to augment the information to complete the PSG picture. We have demonstrated from our SelfAppliedSomnography recordings for A1s (PSG) that the frontal EEG signals (recorded on the forehead) do contain everything that is needed to derive a PSG equivalent sleep staging accuracy. It is therefore possible to record one or two EEG signals using the a recording device, such as a Nox T3s HSAT device, in addition to the regular HSAT signals (the T3s already has those two channels and when this is done, we call it HSAT+) and score the NREM periods into N1, N2 and N3 based on the the EEGs.

A method is provided for augmenting a HSAT study. The method includes recording with a recording device, such as a Nox T3s, a HSAT study with one or two additional frontal-EEG channels. A BodySleep analysis method or system, as described herein, is used to derive all the parameters that can be derived from the BS signals. An auto scoring is run on the EEG signals, determining sleep stages and arousals. The results are combined from both in a relative manner for highest accuracy PSG like outcomes, including PLMS from methode #1 above, sleep staging, sleep time, and combined/optimal arousals.

HSAT+ has been used to score arousals and sleep stages before. However, combining such processing on the EEG with the results from BodySleep2, both providing accurate timing of the NREM periods and all the arousals, to both get the most accurate classification of sleep during NREM and improve the accuracy of the overall sleep staging and arousals, is something new.

### Using a Systems, Sensor, or Method Described Herein (e.g., as Implemented in a Nox BodySleep2.0) for Patient Monitoring

Background: Sleep Care Management is a significant and large business. Compliance to therapy, especially CPAP is very low in an unmanaged model, as it takes training and adaptation to get the therapy right and the patient comfortable. Unmanaged therapy has demonstrated over 50% non-compliance in the first year in multiple studies over the years. A managed therapy has however in Nox Health demonstrated over 90% adherence, thanks to continuous monitoring on performance over radio modules in the CPAP's and high-quality service by care managers. Similar to having a personal trainer for exercise, the care-managers use a preemptive approach to spot patients struggling and adjust their therapy to be effective again. This model however only supports CPAP units with a radio module, but alternative therapies such as oral appliances therapy (OAT), does not support this option. Therefore OAT is rarely used in value based programs, as they are hard to monitor. Using a simple 2 belt sensor device (preferable disposable) that the patient can use to measure one or more nights once or more often during the year, would be sufficient to both help the patient to adjust to alternative therapies like OAT and regularly check the patients compliance.

A sleep care management method is provided wherein one or more RIP belts, preferably two RIP belts and preferably single patient use belts, are provide to a customer on request. An app is provided to the patient to download. The patient downloads an app in his mobile phone that is paired with the sensor for wireless communications (such as BLE). The patient sleeps with the sensor that streams the recorded data either directly or buffered to the mobile device, where it is received and buffered before being streamed to the cloud for storage. The received data is processed by the systems, sensors, and methods described herein (e.g., as implemented in a Nox BodySleep2.0) and the analysis output provided to an adequate person responsible for monitoring the patient sleep performance or treatment compliance.

A second method is provided similar to the above sleep care management method above, except the device is a recorder (like the Nox T3s), that stores the data, the data is then during or after the study uploaded to the cloud OR the data on the device is downloaded on its physical return to the clinic/operation where BodySleep analysis method or system is used for analysing like in the above method.

With this issue of OAT and other alternative therapies being a fact, more and more solutions are surfacing indicated with the potential of being used for this purpose. Commercial products, such as Apple watch or Oura ring have the potential to provide this type of information if they were somehow calibrated to an accurate reference like we described in a prior patent (calibrating SSS with an HASS, U.S. application Ser. No. 17/351,933). This process, of using a BodySleep analyzed data from a simple sensor device with 2 RIP belts (preferable single patient use) would both provide a high accuracy sleep study information from a simple and low cost screener that could secondly by used in combination with a wearable, to calibrate it (according to the HASS/SSS patent application)

### Using BodySleep Sensors for Augmenting CPAP Data for Improved Therapy Management

As described above, sleep care management using CPAP devices is based on data measured by the CPAP and delivered over radio modules to the cloud. The CPAP is however only in an indirect connection to the patient, “sees” the patient through the air-tube between the CPAP and the patient, based on the air-pressure and air-flow signals. Even if this gives the PAP device significant information on how well the patient is breathing, it does not have much to work with regarding how well he sleeps. This is especially difficult, as a patient on an inefficient treatment might not have direct apneas, but is half-cured, meaning that he might be struggling and having arousals. Similar to the ideas described in U.S. application Ser. No. 17/351,933, which is incorporated herein by reference, where we used the high accuracy sleep study recording to calibrate a simplified sleep study (such as for wearable) providing a clinically useful pre- and post-data analysis of the SSS, the same can be done using a BodySleep2 Sensor in association with the CPAP data. A few night BodySleep sensor or system, as described herein, recording during CPAP treatment, both provides the information how well the patient slept during those nights on treatment, but as well information how to interpret the data from the CPAP up to the point of the BodySleep sensor recording and from that time one. This could improve the reliability of the data that the care managers have to work with and provide valuable and early warning if the patient's performance starts to degrade, that would eventually lead to the patient stopping using the therapy.

A method is provided wherein a BodySleep sensor performs a recording on a patient using CPAP, preferable over multiple nights, a BodySleep analysis method is performed. The comparison is used between the received data from the CPAP and the measured data from BodySleep analysis method to augment the accuracy of the CPAP data Post-data analysis is performed on stored CPAP data to determine how the patient has been trending. The calibrated CPAP data model may be used in the future to keep trending the patient with high accuracy and to identify when he is at risk of quitting therapy.

### Combinability of Embodiments and Features

This disclosure provides various examples, embodiments, systems, devices, and methods that predict sleep arousals using non-EEG signal groups. Methods and systems are disclosed herein that predict sleep arousals without requiring EEG signal groups. Also provided, as embodiments, are methods and systems using on an effective AI model tailored for HSAT, that can predict or identify sleep arousals using non-brain signal groups, or in other words using signals not obtained from a brain-machine-interface (BMI). Also provided, as embodiments, are methods and systems using on an effective AI model tailored for HSAT, that can predict sleep arousals using only non-EEG signal groups. And even further are provided, as further embodiments, methods and systems using on an effective AI model tailored for HSAT, that can predict sleep arousals using only two non-EEG signal groups. Unless expressly stated, or unless such examples, embodiments, and features would be mutually exclusive, the various examples, embodiments, and methods disclosed herein should be understood to be combinable with other examples, embodiments, or methods described herein.

In addition to the above, further embodiments and examples include the following groups and enumerated embodiments of systems, devices, and methods that predict sleep arousals using non-EEG signal groups or predict sleep arousals without requiring EEG signal groups.

Methods, Devices, and Systems for for determining an arousal in a sleep study of a subject using one or more body signals.

1. A method for determining an arousal or arousal-associated event in a sleep study of a subject, the method comprising: obtaining data from one or more body signals, the one or more body signals being non-brain signals; and determining an arousal or arousal-associated event of the subject using the data from one or more body signals.

2. The method according to any one or a combination of one or more of 1 above and/or 3-44 below, wherein determining the arousal or arousal-associated event includes using classifier to perform a classification of the of the one or more body signals, wherein the classifier is a neural network, artificial neural network, decision tree or trees, forests of decision trees, clustering, and/or a support vector machine.
3. The method according to any one or a combination of one or more of 1-2 above and/or 4-44 below, wherein the classifier is a convolutional neural network (CNN).
4. The method according to any one or a combination of one or more of 1-3 above and/or 5-44 below, wherein the classifier is a transformer neural network.
5. The method according to any one or a combination of one or more of 1-4 above and/or 6-44 below, wherein the one or more body signals are non-cardiac body signals.
6. The method according to any one or a combination of one or more of 1-5 above and/or 7-44 below, wherein the one or more body signals are indictive of a respiratory activity of the subject.
7. The method according to any one or a combination of one or more of 1-6 above and/or 8-44 below, wherein the one or more body signals include respiratory inductance plethysmography (RIP) signals.
8. The method according to any one or a combination of one or more of 1-7 above and/or 9-44 below, wherein obtaining the one or more body signals include obtaining a thorax effort signal (T), the thorax effort signal (T) being an indicator of a thoracic component of the respiratory effort.
9. The method according to any one or a combination of one or more of 1-8 above and/or 10-44 below, wherein the one or more body signals include respiratory inductance plethysmography (RIP) signals and wherein obtaining the one or more body signals include obtaining an abdomen effort signal (A), the abdomen effort signal (A) being an indicator of an abdominal component of the respiratory effort.
10. The method according to any one or a combination of one or more of 1-9 above and/or 11-44 below, wherein obtaining the one or more body signals includes obtaining a signal of an acceleration signal indicating an acceleration of a body part of the subject.
11. The method according to any one or a combination of one or more of 1-10 above and/or 12-44 below, wherein obtaining the one or more body signals includes obtaining a cardiac signal of the subject.
12. The method according to any one or a combination of one or more of 1-11 above and/or 13-44 below, wherein the one or more body signals are obtained from the subject by non-invasive means.
13. The method according to any one or a combination of one or more of 1-12 above and/or 14-44 below, wherein raw RIP signals are input into the artificial neural network.
14. The method according to any one or a combination of one or more of 1-13 above and/or 15-44 below, wherein predetermined features of the signals are input into the artificial neural network.
15. The method according to any one or a combination of one or more of 1-14 above and/or 16-44 below, further comprising extracting or obtaining a first respiratory component signal from the one or more respiratory signals, the first respiratory component signal being representative of a component of the respiratory activity of the subject.
16. The method according to 15 above, wherein the first respiratory component signal includes an abdomen respiratory volume signal, a thorax respiratory volume signal, the sum of the abdomen and thorax respiratory volume signals (RIPSum), a time derivative of the abdomen respiratory volume signal, a time derivative of the thorax respiratory volume signal, a time derivative of the sum of the abdomen respiratory volume signal and the thorax respiratory volume signal (RIPflow), a respiratory phase signal indicating the phase difference between the abdomen respiratory volume signal and the thorax respiratory volume signal, or a respiratory rate signal (RespRate).
17. The method according to any one or a combination of one or more of 1-16 above and/or 18-44 below, further comprising pre-processing of the one or more respiratory signals before extracting features from the one or more respiratory signals.
18. The method according to any one or a combination of one or more of 1-17 above and/or 19-44 below, wherein the arousal or arousal-associated event of the subject is determined without an EEG signal.
19. A method of estimating an apnea-hypopnea-index (AHI) comprising the method for determining an arousal or arousal-associated event in a sleep study of the subject according to any one or a combination of one or more of 1-18 above or 20-44 below.
20. The method according to any one or a combination of one or more of 1-19 above and/or 21-44 below, comprising performing a prediction for each of a series of time intervals and aggregating the predictions to score an arousal event.
21. The method according to any one or a combination of one or more of 1-20 above and/or 22-44 below, wherein the determining of the arousal or arousal-associated event of the subject is based only a RIP belt signals.
22. The method according to any one or a combination of one or more of 1-21 above and/or 23-44 below, wherein the determining of the arousal or arousal-associated event of the subject is used to aid in the scoring of a respiratory event.
23. The method according to 22 above, wherein the respiratory event is an apnea.
24. The method according to 22 above, wherein the respiratory event is a hypopnea.
25. The method according to 22 above, wherein the respiratory event is a respiratory effort related arousal (RERA).
26. A method for determining an arousal or arousal-associated event in a sleep study of a subject, the method comprising: obtaining data from one or more body signals, the one or more body signals being non-brain signals, the one or more body signals including an oximetry signal; and determining an arousal of the subject using the data from one or more body signals.
27. The method according to any one or a combination of one or more of 1-26 above and/or 28-44 below, wherein determining the arousal or arousal-associated event includes using classifier to perform a classification of the of the one or more body signals, wherein the classifier is a neural network, artificial neural network, decision tree or trees, forests of decision trees, clustering, and/or a support vector machine.
28. The method according to any one or a combination of one or more of 1-27 above and/or 29-44 below, wherein the classifier is a convolutional neural network (CNN).
29. The method according to any one or a combination of one or more of 1-28 above and/or 30-44 below, wherein the classifier is a transformer neural network.
30. The method according to any one or a combination of one or more of 1-29 above and/or 31-44 below, wherein the oximetry is the only body signal used in the determining the arousal or arousal-associated event of the subject.
31. A method of estimating an apnea-hypopnea-index (AHI) of a subject in a sleep study, the method comprising: obtaining data from one or more body signals, the one or more body signals being non-brain signals; and determining an apnea-hypopnea-index (AHI) of the subject using the data from one or more body signals.
32. The method according to any one or a combination of one or more of 1-31 above and/or 33-44 below, wherein determining the arousal or arousal-associated event includes using classifier to perform a classification of the of the one or more body signals, wherein the classifier is a neural network, artificial neural network, decision tree or trees, forests of decision trees, clustering, and/or a support vector machine.
33. The method according to any one or a combination of one or more of 1-32 above and/or 34-44 below, wherein the classifier is a convolutional neural network (CNN).
34. The method according to any one or a combination of one or more of 1-33 above and/or 35-44 below, wherein the classifier is a transformer neural network.
35. The method according to any one or a combination of one or more of 1-34 above and/or 36-44 below, wherein the one or more body signals include respiratory inductance plethysmography (RIP) signals.
36. The method according to any one or a combination of one or more of 1-35 above and/or 37-44 below, wherein the one or more body signals consists of one or more respiratory inductance plethysmography (RIP) signals.
37. The method according to any one or a combination of one or more of 1-36 above and/or 38-44 below, wherein the one or more body signals consists of one or more oximetry (SpO2) signals.
38. A method for determining an apnea in a sleep study of a subject, the method comprising: obtaining data from one or more body signals, the one or more body signals being non-brain signals, and one of the body signals being an abdomen RIP signal and/or a thoracic RIP signal; and determining an apnea of the subject using the data from one or more body signals.
43. A method for determining a hypopnea in a sleep study of a subject, the method comprising: obtaining data from one or more body signals, the one or more body signals being non-brain signals; and one of the body signals being an abdomen RIP signal and/or a thoracic RIP signal; and determining an hypopnea of the subject using the data from one or more body signals.
44. A method for determining a RERA in a sleep study of a subject, the method comprising: obtaining data from one or more body signals, the one or more body signals being non-brain signals, one of the body signals being an abdomen RIP signal and/or a thoracic RIP signal; and determining a RERA of the subject using the data from one or more body signals.
45. The method according to any one or a combination of one or more of 1-44 above, wherein the data from one or more body signals is obtained pre-recorded data from a memory storage, is obtained by receiving the data through a data transmission, and/or is obtained by receiving the data over a network.
46. A hardware storage device having stored thereon computer executable instructions which, when executed by one or more processors of a computer system, configure the computer system to perform the method according to any one or a combination of one or more of 1-45 above.
47. A hardware storage device having stored thereon computer executable instructions which, when executed by one or more processors of a computer system, configure the computer system to perform at least the following: obtain data from one or more body signals, the one or more body signals being non-brain signals; determine an arousal or arousal-associated event of the subject using the data from one or more body signals.
48. A system for determining an arousal or arousal-associated event of a subject, the system comprising: a receiver configured to receive one or more body signals, the one or more body signals being non-brain signals; a memory storage having instructions stored thereon; and the processor configured execute the instructions stored on the memory storage and thereby perform the method according to any one of methods 1-45 above.
49. The system according to any one or a combination of one or more of 48 above and/or 50 below, further comprising a first RIP belt and a second RIP belt.
50. The according to any one or a combination of one or more of 48-49 above, wherein system is configured to obtain the data from the one or more body signals by receiving a transmission, either wireless or by hardwire, of said data.
51. A hardware storage device having stored thereon computer executable instructions which, when executed by one or more processors of a computer system, configure the computer system to perform at least the following: obtain data from one or more body signals, the one or more body signals being non-brain signals; determine an arousal or arousal-associated event of the subject using the data from one or more body signals.
52. The method according to any one or a combination of one or more of 1-45 above, wherein the data from one or more body signals does not include or does not require a signal from a pulse oximeter.
53. The method according to any one or a combination of one or more of 1-45 above, wherein the arousal or arousal-associated event of the subject is determined without a signal from a pulse oximeter.
54. The method according to any one or a combination of one or more of 1-45 and/or 52-53 above, wherein an AHI is derived from RIP signals or one or more RIP belts, wherein the method further comprises: determining arousals and/or arousal-associated events of the subject using the data from the RIP signals; detecting apneas (e.g., based on 90% drop in flow) and potential hypopneas (for example, based on >30% drop); associating the potential hypopneas that have >30%/4% drop in SpO2; associating the potential Hypopneas that are associated with an arousal; deriving the total sleep time; and then based thereon, calculating the AHI for the sleep study in a manner similar to the conventional way an AHI would be calculated as if the sleep study included an EEG signal.
55. The method according to any one or a combination of one or more of 1-45 and/or 52-53 above, wherein an AHI is derived from RIP signals or one or more RIP belts, wherein the method further comprises: directly determining apnea events, wherein an artificial neural network ANN is used to directly determine and output apneas and hypopneas from HSAT signals; determine total sleep time TST and based thereon calculating an AHI.
56. The method according to any one or a combination of one or more of 1-45 and/or 52-55 above, wherein an AHI is derived from RIP signals or one or more RIP belts, wherein the method further comprises directly deriving apnea counting.
57. The method according to any one or a combination of one or more of 1-45 and/or 52-53 above, wherein an AHI is derived from RIP signals or one or more RIP belts, wherein the method further comprises: using an artificial neural network to directly determine and output apneas and/or hypopneas during sleep from HSAT signals.
58. The method according to 57 above, wherein the method does not require or is performed with a step of having independent artificial neural network ANN determining the sleep but classifying sleep in the same round as the apneas.
59. A method for effectively diagnosing other non-respiratory related disorders by determining arousals from RIP signals according to any one or a combination of one or more of 1-45 and/or 52-58 above.
60. The method according to any one or a combination of one or more of 1-45 and/or 52-58 above, using non-EEG signals only.
61. The method according to any one or a combination of one or more of 1-45 and/or 52-58 above, using at least two respiratory band signals, representative of respiratory volume and respiratory flow.
62. The method according to any one or a combination of one or more of 1-45 and/or 52-58, and/or 60-62 above, above, further comprising determining Apnea Hypopnea Index (AHI) where Total Sleep Time required for the calculation of AHI is determined by processing two respiratory band signals only or in combination with other received body signals.
63. The method according to any one or a combination of one or more of 1-45 and/or 52-58, and/or 60-62 above, above, further comprising classifying AHI into periods of REM and NREM, where the Wake/REM/NREM classification is determined by processing the two respiratory signals only or in combination with other received body signals.
64. The method according to any one or a combination of one or more of 1-45 and/or 52-58, and/or 60-63 above, wherein the processing of the two respiratory bands only is sufficiently sensitive to closely resemble apneas and hypopneas or all apneas and hypopneas, including those that based on applicable clinical standards should be classified as desaturation related hypopneas following gold standard PSG rules.
65. The method according to any one or a combination of one or more of 1-45 and/or 52-58, and/or 60-64 above, wherein the processing of the two respiratory bands for detecting arousals is sufficiently sensitive to closely resemble all non-respiratory event related arousals as well as respiratory event related arousals.
66. The method according to any one or a combination of one or more of 1-45 and/or 52-58, and/or 60-65 above, wherein the processing of the available HSAT signals, including the two respiratory bands while excluding the signals from pulse oximetry, is accurate enough to provide AHI for conclusive diagnosis for sleep apnea, (into normal(AHI<=5), mild(5<=AHI<15), moderate (15<=AHI<30) and severe (AHI>=30)).
67. The method according to any one or a combination of one or more of 1-45 and/or 52-58, and/or 60-66 above, wherein the arousal and/or arousal related hypopnea classification is performed by artificial neural network, decision tree or trees, forests of decision trees, clustering, and/or a support vector machine.

Certain terms are used throughout the description and claims to refer to particular methods, features, or components. As those having ordinary skill in the art will appreciate, different persons may refer to the same methods, features, or components by different names. This disclosure does not intend to distinguish between methods, features, or components that differ in name but not function. The figures are not necessarily drawn to scale. Certain features and components herein may be shown in exaggerated scale or in somewhat schematic form and some details of conventional elements may not be shown or described in interest of clarity and conciseness.

As used herein, the following abbreviations carry the following meanings. AASM The American Academy of Sleep Medicine; ADHD Attention deficit hyperactivity disorder; AF Atrial fibrillation; AHI Apnea-Hypopnea index: AI Artificial intelligence; ANN Artificial neural network; ATXAtomoxetine; BDZ Benzodiazepans; BMI Body mass index; BPAP Bi-level positive airway pressure; CAD Coronary artery disease; CNN Convolutional Neural Networks; COPD Chronic obstructive pulmonary disease; CPAP Continuous positive airway pressure; CSA Central sleep apnea; DMD Duchenne muscular dystrophy; EEG Electroencephalogram; EEO Electro-oculogram; HGNS Hypoglossal nerve stimulation; HSAT Home sleep apnea test; HSAT+ Enriched home sleep apnea test; HVR Hypoxic ventilatory response; ISR Inter-scorer reliability: LC Locus coeruleus; MAD Mandibular advancement devices; ML Machine learning; MPH Methylphenidate; NDRI Norepinephrine-Dopamine Reuptake Inhibitor NE Norepinephrine; NREM Non-Rapid Eye Movement; OSA Obstructive sleep apnea: PAP Positive airway pressure; PLMD Periodic limb movement disorder; PLMI Periodic limb movement index; PSG Polysomnography; RCT Randomized controlled trial; REI Respiratory Event Index; REM Rapid eye movement; SARI Serotonin Antagonist and Reuptake Inhibitor; SAS Self-applied somnography; SDB Sleep-disordered breathing; SNRI Serotonin and Norepinephrine Reuptake Inhibitor; SSRI Selective Serotonin Reuptake Inhibitor; SWS Slow-wave sleep; and TST Total Sleep Time.

Although various example embodiments have been described in detail herein, those skilled in the art will readily appreciate in view of the present disclosure that many modifications are possible in the example embodiments without materially departing from the concepts of present disclosure. Accordingly, any such modifications are intended to be included in the scope of this disclosure. Likewise, while the disclosure herein contains many specifics, these specifics should not be construed as limiting the scope of the disclosure or of any of the appended claims, but merely as providing information pertinent to one or more specific embodiments that may fall within the scope of the disclosure and the appended claims. Any described features from the various embodiments disclosed may be employed in combination. In addition, other embodiments of the present disclosure may also be devised which lie within the scopes of the disclosure and the appended claims. Each addition, deletion, and modification to the embodiments that falls within the meaning and scope of the claims is to be embraced by the claims.

Certain embodiments and features may have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges may appear in one or more claims below. Any numerical value is “about” or “approximately” the indicated value, and takes into account experimental error and variations that would be expected by a person having ordinary skill in the art.

1. A method for determining an arousal or an arousal-associated event in a sleep study of a subject, the method comprising:
obtaining data from one or more body signals, the one or more body signals being non-brain signals; and
determining an arousal of the subject using the data from one or more body signals.

2. The method according to claim 1, wherein determining the arousal or arousal-associated event includes using classifier to perform a classification of the of the one or more body signals.

3. The method according to claim 1, wherein the classifier is a neural network, an artificial neural network, a decision tree or trees, forests of decision trees, clustering, a support vector machine, a convolutional neural network (CNN), and/or a transformer neural network.

4. The method according to claim 1, wherein the one or more body signals are respiratory signals.

5. The method according to claim 1, wherein the one or more body signals are non-cardiac body signals.

6. The method according to claim 1, wherein the one or more body signals are indictive of a respiratory activity of the subject.

7. The method according to claim 1, wherein the one or more body signals include respiratory inductance plethysmography (RIP) signals.

8. The method according to claim 7, wherein obtaining the one or more body signals include obtaining a thorax effort signal (T), the thorax effort signal (T) being an indicator of a thoracic component of the respiratory effort, and
obtaining an abdomen effort signal (A), the abdomen effort signal (A) being an indicator of an abdominal component of the respiratory effort.

9. The method according to claim 8, wherein obtaining the one or more body signals further includes obtaining a signal of an acceleration signal indicating an acceleration of a body part of the subject.

10. The method according to claim 1, further comprising performing a prediction for each of a series of time intervals and aggregating the predictions to score an arousal event or an arousal-associated event.

11. The method according to claim 1, wherein the arousal or arousal-associated event of the subject is determined without an EEG signal.

12. A method of estimating an apnea-hypopnea-index (AHI) comprising the method for determining an arousal or an arousal-associated event in a sleep study of the subject according to claim 1.

13. The method according to claim 4, wherein the body signals further comprise an additional body signal that is not a respiratory signal.

14. The method according to claim 1, wherein the determining of the arousal of the subject is based only a RIP belt signals.

15. A hardware storage device having stored thereon computer executable instructions which, when executed by one or more processors of a computer system, configure the computer system to perform the method according to claim 1.

16. A system for determining an arousal or arousal-associated event of a subject, the system comprising:
a receiver configured to receive one or more body signals, the one or more body signals being non-brain signals;
a memory storage having instructions stored thereon; and
a processor configured to perform at least the following:
obtain data from one or more body signals, the one or more body signals being non-brain signals;
determine an arousal or arousal-associated event of the subject using the data from one or more body signals.

17. The system according to claim 16, further comprising a first RIP belt and a second RIP belt.

18. The system according to claim 16, wherein system is configured to obtain the data from the one or more body signals by receiving a transmission, either wireless or by hardwire, of said data.

19. A method of estimating an apnea-hypopnea-index (AHI) of a subject in a sleep study, the method comprising:
obtaining data from one or more body signals, the one or more body signals being non-brain signals; and
determining an apnea-hypopnea-index (AHI) of the subject using the data from one or more body signals.

20. The method according to claim 19, wherein the one or more body signals include respiratory inductance plethysmography (RIP) signals.
